SEQUENCE LISTING (224 sequences)

SEQ ID NO: 1 - DNA, 103 bp, synthetic construct
  source 1..103: mol_type = other DNA; note = Random Pool
ggagctcagccttcactgcnnnnnnnnnnnnnnnnnnnnnnnnnnctgcttcggcagnnnnnnnnnnnnnnnnnnnnnnnnnnggcaccacggtcggatccac

SEQ ID NO: 2 - DNA, 59 bp, synthetic construct
  source 1..59: mol_type = other DNA; note = Random 20F Pool
ggagctcagccttcactgcnnnnnnnnnnnnnnnnnnnnggcaccacggtcggatccac

SEQ ID NO: 3 - DNA, 69 bp, synthetic construct
  source 1..69: mol_type = other DNA; note = Random 30F Pool
ggagctcagccttcactgcnnnnnnnnnnnnnnnnnnnnnnnnnnnnnnggcaccacggtcggatccac

SEQ ID NO: 4 - DNA, 103 bp, synthetic construct
  source 1..103: mol_type = other DNA; note = Dope seqA Pool
ggagctcagccttcactgccgcccccaccgggtttgaaaacctggctgcttcggcagttgtatcctttggggctcggcaattcggcaccacggtcggatccac

SEQ ID NO: 5 - DNA, 103 bp, synthetic construct
  source 1..103: mol_type = other DNA; note = Dope seqB Pool
ggagctcagccttcactgcaagatgtggaccatttaacttgtagactgcttcggcaggcggctgttccctcaagggaacgcttggcaccacggtcggatccac

SEQ ID NO: 6 - DNA, 103 bp, synthetic construct
  source 1..103: mol_type = other DNA; note = Dope seqC Pool
ggagctcagccttcactgccagaccgcgtttagaaacgcgtaaatctgcttcggcagattgattactatcgatctggtaacgaggcaccacggtcggatccac

SEQ ID NO: 7 - RNA, 53 nt, synthetic construct
  source 1..53: mol_type = other RNA; note = SiR-cRNA
gctgcgacgtttgaaaacgtctaactgcttcggcagaacggtatcccggcggc

SEQ ID NO: 8 - skipped

SEQ ID NO: 9 - DNA, 38 bp, synthetic construct
  source 1..38: mol_type = other DNA; note = Primer A
tctaatacgactcactataggagctcagccttcactgc

SEQ ID NO: 10 - DNA, 20 bp, synthetic construct
  source 1..20: mol_type = other DNA; note = Primer B
gtggatccgaccgtggtgcc

SEQ ID NO: 11 - DNA, 33 bp, synthetic construct
  source 1..33: mol_type = other DNA; note = SS_F_v1
acacgacgctcttccgatctcgaccgtggtgcc

SEQ ID NO: 12 - DNA, 34 bp, synthetic construct
  source 1..34: mol_type = other DNA; note = ES_F1_v1
acacgacgctcttccgatctacgaccgtggtgcc

SEQ ID NO: 13 - DNA, 35 bp, synthetic construct
  source 1..35: mol_type = other DNA; note = ES_F2_v1
acacgacgctcttccgatcttacgaccgtggtgcc

SEQ ID NO: 14 - DNA, 36 bp, synthetic construct
  source 1..36: mol_type = other DNA; note = ES_F3_v1
acacgacgctcttccgatctgtacgaccgtggtgcc

SEQ ID NO: 15 - DNA, 37 bp, synthetic construct
  source 1..37: mol_type = other DNA; note = ES_F4_v1
acacgacgctcttccgatctccatcgaccgtggtgcc

SEQ ID NO: 16 - DNA, 38 bp, synthetic construct
  source 1..38: mol_type = other DNA; note = ES_F5_v1
acacgacgctcttccgatctacttacgaccgtggtgcc

SEQ ID NO: 17 - DNA, 39 bp, synthetic construct
  source 1..39: mol_type = other DNA; note = ES_F6_v1
acacgacgctcttccgatctgtacttcgaccgtggtgcc

SEQ ID NO: 18 - DNA, 37 bp, synthetic construct
  source 1..37: mol_type = other DNA; note = SS_R_v1
agacgtgtgctcttccgatctgctcagccttcactgc

SEQ ID NO: 19 - DNA, 38 bp, synthetic construct
  source 1..38: mol_type = other DNA; note = ES_R1_v1
agacgtgtgctcttccgatcttgctcagccttcactgc

SEQ ID NO: 20 - DNA, 39 bp, synthetic construct
  source 1..39: mol_type = other DNA; note = ES_R2_v1
agacgtgtgctcttccgatctatgctcagccttcactgc

SEQ ID NO: 21 - DNA, 40 bp, synthetic construct
  source 1..40: mol_type = other DNA; note = ES_R3_v1
agacgtgtgctcttccgatctcatgctcagccttcactgc

SEQ ID NO: 22 - DNA, 41 bp, synthetic construct
  source 1..41: mol_type = other DNA; note = ES_R4_v1
agacgtgtgctcttccgatctgcatgctcagccttcactgc

SEQ ID NO: 23 - DNA, 42 bp, synthetic construct
  source 1..42: mol_type = other DNA; note = ES_R5_v1
agacgtgtgctcttccgatctagcatgctcagccttcactgc

SEQ ID NO: 24 - DNA, 43 bp, synthetic construct
  source 1..43: mol_type = other DNA; note = ES_R6_v1
agacgtgtgctcttccgatctcatactgctcagccttcactgc

SEQ ID NO: 25 - DNA, 31 bp, synthetic construct
  source 1..31: mol_type = other DNA; note = SS_F_v2
acacgacgctcttccgatctaccgtggtgcc

SEQ ID NO: 26 - DNA, 32 bp, synthetic construct
  source 1..32: mol_type = other DNA; note = ES_F1_v2
acacgacgctcttccgatctcaccgtggtgcc

SEQ ID NO: 27 - DNA, 33 bp, synthetic construct
  source 1..33: mol_type = other DNA; note = ES_F2_v2
acacgacgctcttccgatcttaaccgtggtgcc

SEQ ID NO: 28 - DNA, 34 bp, synthetic construct
  source 1..34: mol_type = other DNA; note = ES_F3_v2
acacgacgctcttccgatctggtaccgtggtgcc

SEQ ID NO: 29 - DNA, 35 bp, synthetic construct
  source 1..35: mol_type = other DNA; note = ES_F4_v2
acacgacgctcttccgatctctataccgtggtgcc

SEQ ID NO: 30 - DNA, 36 bp, synthetic construct
  source 1..36: mol_type = other DNA; note = ES_F5_v2
acacgacgctcttccgatctacgtaaccgtggtgcc

SEQ ID NO: 31 - DNA, 37 bp, synthetic construct
  source 1..37: mol_type = other DNA; note = ES_F6_v2
acacgacgctcttccgatctgtacttaccgtggtgcc

SEQ ID NO: 32 - DNA, 35 bp, synthetic construct
  source 1..35: mol_type = other DNA; note = SS_R_v2
agacgtgtgctcttccgatcttcagccttcactgc

SEQ ID NO: 33 - DNA, 36 bp, synthetic construct
  source 1..36: mol_type = other DNA; note = ES_R1_v2
agacgtgtgctcttccgatctttcagccttcactgc

SEQ ID NO: 34 - DNA, 37 bp, synthetic construct
  source 1..37: mol_type = other DNA; note = ES_R2_v2
agacgtgtgctcttccgatctattcagccttcactgc

SEQ ID NO: 35 - DNA, 38 bp, synthetic construct
  source 1..38: mol_type = other DNA; note = ES_R3_v2
agacgtgtgctcttccgatctcaatcagccttcactgc

SEQ ID NO: 36 - DNA, 39 bp, synthetic construct
  source 1..39: mol_type = other DNA; note = ES_R4_v2
agacgtgtgctcttccgatctgcgttcagccttcactgc

SEQ ID NO: 37 - DNA, 40 bp, synthetic construct
  source 1..40: mol_type = other DNA; note = ES_R5_v2
agacgtgtgctcttccgatctagcattcagccttcactgc

SEQ ID NO: 38 - DNA, 41 bp, synthetic construct
  source 1..41: mol_type = other DNA; note = ES_R6_v2
agacgtgtgctcttccgatctcatgcttcagccttcactgc

SEQ ID NO: 39 - DNA, 134 bp, synthetic construct
  source 1..134: mol_type = other DNA; note = PhiX_v1
acacgacgctcttccgatctnnnnnnngaaaaannnnnnnnnnnnnnnnnnnnnnnnnntttttgtttttgnnnnnnnnnnnnnnnnnnnnnnnnnngcactcaagnnnnnnnagatcggaagagcacacgtct

SEQ ID NO: 40 - DNA, 130 bp, synthetic construct
  source 1..130: mol_type = other DNA; note = PhiX_v2
acacgacgctcttccgatctnnnnngaaaaannnnnnnnnnnnnnnnnnnnnnnnnntttttgtttttgnnnnnnnnnnnnnnnnnnnnnnnnnngccctcaagnnnnnagatcggaagagcacacgtct

SEQ ID NO: 41 - RNA, 103 nt, synthetic construct
  source 1..103: mol_type = other RNA; note = RLB1
ggagctcagccttcactgcacggaaattccacaaggaaaatccgactgcttcggcaattcaagtcttgaattactgtttgtcgggcaccacggtcggatccac

SEQ ID NO: 42 - RNA, 102 nt, synthetic construct
  source 1..102: mol_type = other RNA; note = RLB2
ggagctcagccttcactgctagctgcggattcaagtcttgacatactgttcggcagcaaacagtaggaggttaaacgatcttggcaccacggtcggatccac

SEQ ID NO: 43 - RNA, 103 nt, synthetic construct
  source 1..103: mol_type = other RNA; note = RLB3
ggagctcagccttcactgcgacgtttgaaaacgtctaacacgagtctgcttcggcagagtctgacggtatcccggcggatgttggcaccacggtcggatccac

SEQ ID NO: 44 - RNA, 103 nt, synthetic construct
  source 1..103: mol_type = other RNA; note = RLB4
ggagctcagccttcactgcttggtacactgttaaggatatctctactgcttcggcagacggtttgaaaaccgttaatacagttggcaccacggtcggatccac

SEQ ID NO: 45 - RNA, 103 nt, synthetic construct
  source 1..103: mol_type = other RNA; note = RLB5
ggagctcagccttcactgcgtggcttcgttatgacatcgatatatctgcttcggcaggcctaggtgcctttctcgatgcttggggcaccacggtcggatccac

SEQ ID NO: 46 - RNA, 100 nt, synthetic construct
  source 1..100: mol_type = other RNA; note = RLB6
ggagctcagccttcactgcgtcgtcgattcaagtcttgacttactgttcggcagaactgcatgtgaaaaacagttcccgcggcaccacggtcggatccac

SEQ ID NO: 47 - RNA, 102 nt, synthetic construct
  source 1..102: mol_type = other RNA; note = RLB7
ggagctcagccttcactgcgttctatcggtaatacagtttgaaaactgcttcgcagtcgtagcacgacccaacgcttgctccggcaccacggtcggatccac

SEQ ID NO: 48 - RNA, 103 nt, synthetic construct
  source 1..103: mol_type = other RNA; note = RLB8
ggagctcagccttcactgccgagcattgcaagtcttgacttactgctgcttcggcagtccgtagtgttgcctatggtcgccagggcaccacggtcggatccac

SEQ ID NO: 49 - RNA, 103 nt, synthetic construct
  source 1..103: mol_type = other RNA; note = RLB9
ggagctcagccttcactgcaggatattacgcttgactacgtgttcctgcttcggcagtgtgtgacgagcactgacgacctctcggcaccacggtcggatccac

SEQ ID NO: 50 - RNA, 103 nt, synthetic construct
  source 1..103: mol_type = other RNA; note = RLB10
ggagctcagccttcactgcagcgagatactcttgataaagtccgtctgcttcggcaggttagtagcttatgccgttgtgtcgtggcaccacggtcggatccac

SEQ ID NO: 51 - RNA, 102 nt, synthetic construct
  source 1..102: mol_type = other RNA; note = RLB11
ggagctcagccttcactgcactacccaatgttacgcttgactacgtgcttcggcagtccatggaggcattaaccaccgttgaggcaccacggtcggatccac

SEQ ID NO: 52 - RNA, 103 nt, synthetic construct
  source 1..103: mol_type = other RNA; note = RLB12
ggagctcagccttcactgctgaccgattcaatgcagtgaacggcactgcttcggcagtccgggtgtcgtggataggctataacggcaccacggtcggatccac

SEQ ID NO: 53 - RNA, 103 nt, synthetic construct
  source 1..103: mol_type = other RNA; note = RLB13
ggagctcagccttcactgctggctcgatcgtccatagctctagagctgcttcggcaaaggtcctcttgataaagtcaagccgaggcaccacggtcggatccac

SEQ ID NO: 54 - RNA, 103 nt, synthetic construct
  source 1..103: mol_type = other RNA; note = RLB14
ggagctcagccttcactgccaggctaggcttggccccatttttacctgcttcggcaggacgcccgtgtgtgaatataaaccctggcaccacggtcggatccac

SEQ ID NO: 55 - RNA, 101 nt, synthetic construct
  source 1..101: mol_type = other RNA; note = RLB15
ggagctcagccttcactgcagtaatgttgaaacaggacgtcttctgcttcggcagagattaggttatcaccctgtggggaaggcaccacggtcggatccac

SEQ ID NO: 56 - RNA, 102 nt, synthetic construct
  source 1..102: mol_type = other RNA; note = RLB16
ggagctcagccttcactgcatggaagctggactcgtaccgtttgctgcttcggcaggtatcgtctacgtgctagcttggctaggcaccacggtcggatccac

SEQ ID NO: 57 - RNA, 103 nt, synthetic construct
  source 1..103: mol_type = other RNA; note = RLB17
ggagctcagccttcactgcgcgcgcagtacctgccacttggggaactgcttcggcagttgtgcgcgaagtcctggccgcggttggcaccacggtcggatccac

SEQ ID NO: 58 - RNA, 103 nt, synthetic construct
  source 1..103: mol_type = other RNA; note = RLB18
ggagctcagccttcactgcctcggtcgaaagtaagtcttgacatactgcttcggcagaggcgacgcttgaccgtgaacactaaggcaccacggtcggatccac

SEQ ID NO: 59 - RNA, 102 nt, synthetic construct
  source 1..102: mol_type = other RNA; note = RLB19
ggagctcagccttcactgcttggccgaacacagatccatctgaacctgcctcggcagctctttcactaatcgacgacccgttggcaccacggtcggatccac

SEQ ID NO: 60 - RNA, 103 nt, synthetic construct
  source 1..103: mol_type = other RNA; note = RLB20
ggagctcagccttcactgcattcaacgaattcaagtcttgatatactgtttcggcagtcgtccggtcactcggatgtatagctggcaccacggtcggatccac

SEQ ID NO: 61 - RNA, 103 nt, synthetic construct
  source 1..103: mol_type = other RNA; note = RLB21
ggagctcagccttcactgcgcagaggtccgcttgaaaacgtcctgctgcttcggcagctccctcaccgtggtcgtgcactgccggcaccacggtcggatccac

SEQ ID NO: 62 - RNA, 103 nt, synthetic construct
  source 1..103: mol_type = other RNA; note = RLB22
ggagctcagccttcactgcatcgtggcgcgtgactcgtgacaactctgcttcggcaggtagtgcgaggttggccttgagccttggcaccacggtcggatccac

SEQ ID NO: 63 - RNA, 102 nt, synthetic construct
  source 1..102: mol_type = other RNA; note = RLB23
ggagctcagccttcactgcgagtggaccaagtcttgacttactggctgcttggcagtgagacttatgtgagccttaaccgtgggcaccacggtcggatccac

SEQ ID NO: 64 - RNA, 103 nt, synthetic construct
  source 1..103: mol_type = other RNA; note = RLB24
ggagctcagccttcactgctgactgctcgaatggccgtgatgcgactgcttcggcagtctgggttgcgtgctcccgcttgtcgggcaccacggtcggatccac

SEQ ID NO: 65 - RNA, 103 nt, synthetic construct
  source 1..103: mol_type = other RNA; note = RLB25
ggagctcagccttcactgctaggtccgcttgataacgtcagcagtctgcttcggcaggtggttgagatctgtcgcgagcccttggcaccacggtcggatccac

SEQ ID NO: 66 - RNA, 103 nt, synthetic construct
  source 1..103: mol_type = other RNA; note = RLB26
ggagctcagccttcactgctccatggccttttcccttgagtagctctgcttcggcagatattgtatcttgaacacctacccaaggcaccacggtcggatccac

SEQ ID NO: 67 - RNA, 103 nt, synthetic construct
  source 1..103: mol_type = other RNA; note = RLB29
ggagctcagccttcactgctgaggtgtgttatgcttgactacatgccgcttcggcagtcggttcggtcctccattttgcgtgcggcaccacggtcggatccac

SEQ ID NO: 68 - RNA, 103 nt, synthetic construct
  source 1..103: mol_type = other RNA; note = RLB30
ggagctcagccttcactgcgatcaggtccgcttgataacgtcgatctgcttctgaaggatgatccttactctctcttttgatcggcaccacggtcggatccac

SEQ ID NO: 69 - RNA, 103 nt, synthetic construct
  source 1..103: mol_type = other RNA; note = RLB31
ggagctcagccttcactgcccggtgacggtaagactgactcctcactgcttcggcaggctgctggtagcacctcttgacaaagggcaccacggtcggatccac

SEQ ID NO: 70 - RNA, 103 nt, synthetic construct
  source 1..103: mol_type = other RNA; note = RLB32
ggagctcagccttcactgcactcacgaatagcaagtcttgatatactgcttcggcagcgcaggcgaaaagcacccgtaccgcaggcaccacggtcggatccac

SEQ ID NO: 71 - RNA, 102 nt, synthetic construct
  source 1..102: mol_type = other RNA; note = RLB33
ggagctcagccttcactgcggaaagtccaggaatcgcattaacctctgcttcggcagatgtgtgtgtcaactcgtttggcctggcaccacggtcggatccac

SEQ ID NO: 72 - RNA, 103 nt, synthetic construct
  source 1..103: mol_type = other RNA; note = RLB36
ggagctcagccttcactgctagctgcggattcaagtcttgacatactgttcggcagcaaaacagtaggaggttaaacgatcttggcaccacggtcggatccac

SEQ ID NO: 73 - RNA, 102 nt, synthetic construct
  source 1..102: mol_type = other RNA; note = RLB37
ggagctcagccttcactgctaatagcatggtccgcttgactacgtctgcttcggcagtggctgatttacctgtgatcgtgcgggcaccacggtcggatccac

SEQ ID NO: 74 - RNA, 99 nt, synthetic construct
  source 1..99: mol_type = other RNA; note = RLB41
ggagctcagccttcactgctccacctgtggtgacctgtcctctgcttcggcagagtgacttcaaagcgcttgaaaacgaggcaccacggtcggatccac

SEQ ID NO: 75 - RNA, 101 nt, synthetic construct
  source 1..101: mol_type = other RNA; note = RLB42
ggagctcagccttcactgcgtcgtcgattcaagtcttgacttactgttcggcagaactgcatgtgaaaaaacagttcccgcggcaccacggtcggatccac

SEQ ID NO: 76 - RNA, 104 nt, synthetic construct
  source 1..104: mol_type = other RNA; note = RLB44
ggagctcagccttcactgcagtaggtatcctgagcctcagatcgtgctgcttcggcagatcgtgcaccaagtcttgaattactgggcaccacggtcggatccac

SEQ ID NO: 77 - RNA, 103 nt, synthetic construct
  source 1..103: mol_type = other RNA; note = RLB45
ggagctcagccttcactgcggtagtcgtttagcgtgatggttatgccgcttcggcagtggttgcacttacgcttgaatacgtgggcaccacggtcggatccac

SEQ ID NO: 78 - RNA, 103 nt, synthetic construct
  source 1..103: mol_type = other RNA; note = RLB50
ggagctcagccttcactgcactagacctatgccgatgtaagtactctgcttcggcagtcctttcagagtcttgaggactacccggcaccacggtcggatccac

SEQ ID NO: 79 - RNA, 103 nt, synthetic construct
  source 1..103: mol_type = other RNA; note = RLB51
ggagctcagccttcactgcatgttacgcttgactacgtgctgcagctgcttcggcagttccaatcgtgtggactgagcaagtaggcaccacggtcggatccac

SEQ ID NO: 80 - RNA, 102 nt, synthetic construct
  source 1..102: mol_type = other RNA; note = RLB54
ggagctcagccttcactgctttgcaagcttccgcttggcaacgagctgctcggcagtgcagcctcttctgctctgaccgtctggcaccacggtcggatccac

SEQ ID NO: 81 - RNA, 103 nt, synthetic construct
  source 1..103: mol_type = other RNA; note = RLB63
ggagctcagccttcactgcattcaagtcttgaactactgttgcagctgcttcggcagaagggttctttggttcaacccgcggaggcaccacggtcggatccac

SEQ ID NO: 82 - RNA, 102 nt, synthetic construct
  source 1..102: mol_type = other RNA; note = RLB70
ggagctcagccttcactgccctctcaccgatacgcttttcacccgctgcttcggcgccatagcaagtcttgacttactgcgtggcaccacggtcggatccac

SEQ ID NO: 83 - RNA, 102 nt, synthetic construct
  source 1..102: mol_type = other RNA; note = RLB82
ggagctcagccttcactgcgactgatttggagcctaatgtatagtctgcttggcagacttattcagcgttaccgtacattgtggcaccacggtcggatccac

SEQ ID NO: 84 - RNA, 103 nt, synthetic construct
  source 1..103: mol_type = other RNA; note = RLB95
ggagctcagccttcactgctgcgtcgaatcgcaagtcttgacctactgcttcgacagtacagggagctgttccgctgcgccgtggcaccacggtcggatccac

SEQ ID NO: 85 - RNA, 102 nt, synthetic construct
  source 1..102: mol_type = other RNA; note = RLB117
ggagctcagccttcactgcattgccgaatccaagtcttgaattactgcttcggcagcgctatctagctccaccgttgaacttggcaccacggtcggatccac

SEQ ID NO: 86 - RNA, 102 nt, synthetic construct
  source 1..102: mol_type = other RNA; note = RLB118
ggagctcagccttcactgccaatgtatggtccgcttgacaacgtctgcttcggcagtgaactcccaccaccgcgcaccctggggcaccacggtcggatccac

SEQ ID NO: 87 - RNA, 103 nt, synthetic construct
  source 1..103: mol_type = other RNA; note = RLB136
ggagctcagccttcactgcaagtcttgacttactgcgtggagatgctgcttcggcagagacgcggtaatgaccgaccatcgttggcaccacggtcggatccac

SEQ ID NO: 88 - RNA, 103 nt, synthetic construct
  source 1..103: mol_type = other RNA; note = NLB1
ggagctcagccttcactgcgtggtgtatagttcctgcgatggcatctgcttcggcagatatactgggatccgtgacgatcatcggcaccacggtcggatccac

SEQ ID NO: 89 - RNA, 103 nt, synthetic construct; identical to SEQ ID NO: 67
  source 1..103: mol_type = other RNA; note = NLB2

SEQ ID NO: 90 - RNA, 103 nt, synthetic construct; identical to SEQ ID NO: 45
  source 1..103: mol_type = other RNA; note = NLB3

SEQ ID NO: 91 - RNA, 103 nt, synthetic construct
  source 1..103: mol_type = other RNA; note = NLB4
ggagctcagccttcactgcgtggtgtatagctcctgcgatggcatctgcttcggcagatatcctgggatccgtgacgatcatcggcaccacggtcggatccac

SEQ ID NO: 92 - RNA, 103 nt, synthetic construct; identical to SEQ ID NO: 81
  source 1..103: mol_type = other RNA; note = NLB5

SEQ ID NO: 93 - RNA, 103 nt, synthetic construct; identical to SEQ ID NO: 41
  source 1..103: mol_type = other RNA; note = NLB6

SEQ ID NO: 94 - RNA, 103 nt, synthetic construct
  source 1..103: mol_type = other RNA; note = NLB7
ggagctcagccttcactgcatcgccgaaaagcaagtcttgaattactacttcggcagaccgtacctgtatccggtctaagtgtggcaccacggtcggatccac

SEQ ID NO: 95 - RNA, 103 nt, synthetic construct; identical to SEQ ID NO: 49
  source 1..103: mol_type = other RNA; note = NLB8

SEQ ID NO: 96 - RNA, 104 nt, synthetic construct; identical to SEQ ID NO: 76
  source 1..104: mol_type = other RNA; note = NLB9

SEQ ID NO: 97 - RNA, 103 nt, synthetic construct
  source 1..103: mol_type = other RNA; note = NLB10
ggagctcagccttcactgctgaggtgtgttatgcttgactacatgccgcttcggcagtcggttcggtcctccattttgcgtgtggcaccacggtcggatccac

SEQ ID NO: 98 - RNA, 103 nt, synthetic construct; identical to SEQ ID NO: 43
  source 1..103: mol_type = other RNA; note = NLB11

SEQ ID NO: 99 - RNA, 103 nt, synthetic construct
  source 1..103: mol_type = other RNA; note = NLB12
ggagctcagccttcactgcgtggcgtatagctcctgcgatggcatctgcttcggcagatatactgggatccgtgacgatcatcggcaccacggtcggatccac

SEQ ID NO: 100 - RNA, 105 nt, synthetic construct
  source 1..105: mol_type = other RNA; note = NLB13
ggagctcagccttcactgcgtgtgctcttccgatctttcagccttcactgctgcggaaatcggcttggcgttgatcatatcctgtggcaccacggtcggatccac

SEQ ID NO: 101 - RNA, 103 nt, synthetic construct
  source 1..103: mol_type = other RNA; note = NLB14
ggagctcagccttcactgcaggtgggcagttagcattggctaatgctccttcggcagacgttgtgacctaagcttgacatcttggcaccacggtcggatccac

SEQ ID NO: 102 - RNA, 103 nt, synthetic construct
  source 1..103: mol_type = other RNA; note = NLB15
ggagctcagccttcactgcgtgatgtatagccccagtgaactatcctgcttcggcagacatatgctccggtccgccgggcatcggcaccacggtcggatccac

SEQ ID NO: 103 - RNA, 103 nt, synthetic construct
  source 1..103: mol_type = other RNA; note = NLB16
ggagctcagccttcactgctcagaaacaggtccgcttgaatacgtctgtttcggcagggtaaccgcgggctaccactcgtgttggcaccacggtcggatccac

SEQ ID NO: 104 - RNA, 103 nt, synthetic construct
  source 1..103: mol_type = other RNA; note = NLB17
ggagctcagccttcactgcagctggcgtggtgtatagtctcctggctgcttcgacagctgtttaaatcgatctggcggacattggcaccacggtcggatccac

SEQ ID NO: 105 - RNA, 106 nt, synthetic construct
  source 1..106: mol_type = other RNA; note = NLB18
ggagctcagccttcactgcgtgctcttccgatctgcgttcagccttcactgctgcggaaatcggcttggcgttgatcatatcctgtggcaccacggtcggatccac

SEQ ID NO: 106 - RNA, 100 nt, synthetic construct; identical to SEQ ID NO: 46
  source 1..100: mol_type = other RNA; note = NLB19

SEQ ID NO: 107 - RNA, 102 nt, synthetic construct; identical to SEQ ID NO: 47
  source 1..102: mol_type = other RNA; note = NLB20

SEQ ID NO: 108 - RNA, 102 nt, synthetic construct; identical to SEQ ID NO: 42
  source 1..102: mol_type = other RNA; note = NLB21

SEQ ID NO: 109 - RNA, 103 nt, synthetic construct; identical to SEQ ID NO: 78
  source 1..103: mol_type = other RNA; note = NLB22

SEQ ID NO: 110 - RNA, 103 nt, synthetic construct; identical to SEQ ID NO: 57
  source 1..103: mol_type = other RNA; note = NLB23

SEQ ID NO: 111 - RNA, 99 nt, synthetic construct
  source 1..99: mol_type = other RNA; note = NLB24
ggagctcagccttcactgcgtggtgtatagctcctgcgatggccagcttcggctgatactgggatccgtgacgatcatcggcaccacggtcggatccac

SEQ ID NO: 112 - RNA, 103 nt, synthetic construct
  source 1..103: mol_type = other RNA; note = NLB25
ggagctcagccttcactgctcttccgatctagcattcagccttcactgctgcggaaatcggcttggcgttgatcatatcctgtggcaccacggtcggatccac

SEQ ID NO: 113 - RNA, 106 nt, synthetic construct
  source 1..106: mol_type = other RNA; note = NLB26
ggagctcagccttcactgcacgtgtgctcttccgatcttcagccttcactgctgcggaaatcggcttggcgttgatcatatcctgtggcaccacggtcggatccac

SEQ ID NO: 114 - RNA, 103 nt, synthetic construct
  source 1..103: mol_type = other RNA; note = NLB27
ggagctcagccttcactgcaagatgtggaccatttaacttgtagactgcttcggcaggcggctgttccctcaagggaacgcttggcaccacggtcggatccac

SEQ ID NO: 115 - RNA, 102 nt, synthetic construct; identical to SEQ ID NO: 51
  source 1..102: mol_type = other RNA; note = NLB28

SEQ ID NO: 116 - RNA, 103 nt, synthetic construct
  source 1..103: mol_type = other RNA; note = NLB29
ggagctcagccttcactgctctagcacgttaagcttgactacttgctgcttcggcagatgttgctgcattactaccgattgttggcaccacggtcggatccac

SEQ ID NO: 117 - RNA, 102 nt, synthetic construct; identical to SEQ ID NO: 86
  source 1..102: mol_type = other RNA; note = NLB30

SEQ ID NO: 118 - RNA, 102 nt, synthetic construct
  source 1..102: mol_type = other RNA; note = NLB31
ggagctcagccttcactgctgagtgtgttatgcttgactacatgccgcttcggcagtcggttcggtcctccattttgcgtgcggcaccacggtcggatccac

SEQ ID NO: 119 - RNA, 103 nt, synthetic construct; identical to SEQ ID NO: 65
  source 1..103: mol_type = other RNA; note = NLB32

SEQ ID NO: 120 - RNA, 102 nt, synthetic construct; identical to SEQ ID NO: 80
  source 1..102: mol_type = other RNA; note = NLB33

SEQ ID NO: 121 - RNA, 103 nt, synthetic construct; identical to SEQ ID NO: 53
  source 1..103: mol_type = other RNA; note = NLB34

SEQ ID NO: 122 - RNA, 101 nt, synthetic construct; identical to SEQ ID NO: 55
  source 1..101: mol_type = other RNA; note = NLB35

SEQ ID NO: 123 - RNA, 102 nt, synthetic construct; identical to SEQ ID NO: 56
  source 1..102: mol_type = other RNA; note = NLB36

SEQ ID NO: 124 - RNA, 103 nt, synthetic construct; identical to SEQ ID NO: 44
  source 1..103: mol_type = other RNA; note = NLB37

SEQ ID NO: 125 - RNA, 103 nt, synthetic construct; identical to SEQ ID NO: 50
  source 1..103: mol_type = other RNA; note = NLB38

SEQ ID NO: 126 - RNA, 103 nt, synthetic construct; identical to SEQ ID NO: 79
  source 1..103: mol_type = other RNA; note = NLB39

SEQ ID NO: 127 - RNA, 103 nt, synthetic construct; identical to SEQ ID NO: 58
  source 1..103: mol_type = other RNA; note = NLB40

SEQ ID NO: 128 - RNA, 103 nt, synthetic construct
  source 1..103: mol_type = other RNA; note = NLB41
ggagctcagccttcactgcaagtcttgatatactgctgtcggctactgcttcggcagctaaccggagtccattgacgtcgatgggcaccacggtcggatccac

SEQ ID NO: 129 - RNA, 102 nt, synthetic construct
  source 1..102: mol_type = other RNA; note = NLB42
ggagctcagccttcactgcttagtgccgtcgagttatcctcataactgcttggcagtatcctcgcacataagtgcgttggtcggcaccacggtcggatccac

SEQ ID NO: 130 - RNA, 103 nt, synthetic construct; identical to SEQ ID NO: 48
  source 1..103: mol_type = other RNA; note = NLB43

SEQ ID NO: 131 - RNA, 103 nt, synthetic construct
  source 1..103: mol_type = other RNA; note = NLB44
ggagctcagccttcactgcttccgaacagggcgaagcgaatccgactgcttcgacagctcaagtcttgatttactgtctgtctggcaccacggtcggatccac

SEQ ID NO: 132 - RNA, 103 nt, synthetic construct
  source 1..103: mol_type = other RNA; note = NLB45
ggagctcagccttcactgcatgaggtccgcttgataacgtccatgctgcttcggcagagttcggcagggtatctatgtgccctggcaccacggtcggatccac

SEQ ID NO: 133 - RNA, 103 nt, synthetic construct; identical to SEQ ID NO: 52
  source 1..103: mol_type = other RNA; note = NLB46

SEQ ID NO: 134 - RNA, 103 nt, synthetic construct; identical to SEQ ID NO: 54
  source 1..103: mol_type = other RNA; note = NLB47

SEQ ID NO: 135 - RNA, 103 nt, synthetic construct
  source 1..103: mol_type = other RNA; note = NLB48
ggagctcagccttcactgccaatcgtcctgaccgccattgggtagctgcttcggcagaggcgcccgtttcaatccacttgttgggcaccacggtcggatccac

SEQ ID NO: 136 - RNA, 106 nt, synthetic construct
  source 1..106: mol_type = other RNA; note = NLB49
ggagctcagccttcactgcgtgtgctcttccgatctattcagccttcactgctgcggaaatcggcttggcgttgatcatatcctgtggcaccacggtcggatccac

SEQ ID NO: 137 - RNA, 106 nt, synthetic construct
  source 1..106: mol_type = other RNA; note = NLB50
ggagctcagccttcactgctgtgctcttccgatctcaatcagccttcactgctgcggaaatcggcttggcgttgatcatatcctgtggcaccacggtcggatccac

SEQ ID NO: 138 - RNA, 103 nt, synthetic construct
  source 1..103: mol_type = other RNA; note = NLB51
ggagctcagccttcactgcagaggagtataccgaggcagcacccgctgcttcggcaggacaaactgtatggccctcgtgcgttggcaccacggtcggatccac

SEQ ID NO: 139 - RNA, 102 nt, synthetic construct
  source 1..102: mol_type = other RNA; note = NLB52
ggagctcagccttcactgcacatcacattagttgacacgtgaagcctgcttcgcaggtgcttaggtccgcttgaaaacgtcaggcaccacggtcggatccac

SEQ ID NO: 140 - RNA, 102 nt, synthetic construct; identical to SEQ ID NO: 59
  source 1..102: mol_type = other RNA; note = NLB53

SEQ ID NO: 141 - RNA, 103 nt, synthetic construct
  source 1..103: mol_type = other RNA; note = NLB54
ggagctcagccttcactgcaatttccgtttgaaaacgggttaatactgcttcggcagtatgtcagtctgacattgcagctcccggcaccacggtcggatccac

SEQ ID NO: 142 - RNA, 103 nt, synthetic construct
  source 1..103: mol_type = other RNA; note = NLB55
ggagctcagccttcactgcaaggccatggtctgcgctcccacgtgctgcttcagcagagcaagtcttgacctactacctgttgggcaccacggtcggatccac

SEQ ID NO: 143 - RNA, 103 nt, synthetic construct
  source 1..103: mol_type = other RNA; note = NLB56
ggagctcagccttcactgcgtggtgtatagctcctgcgatggcatctgcttcggcagatatactgggatccgtgacgatcatcggcaccacggtcggatccac

SEQ ID NO: 144 - RNA, 102 nt, synthetic construct; identical to SEQ ID NO: 83
  source 1..102: mol_type = other RNA; note = NLB57

SEQ ID NO: 145 - RNA, 102 nt, synthetic construct
  source 1..102: mol_type = other RNA; note = NLB58
ggagctcagccttcactgctgaggtgtgttatgcttgactacatgccgcttcggcagtcggttcggtcctccattttggtgcggcaccacggtcggatccac

SEQ ID NO: 146 - RNA, 104 nt, synthetic construct
  source 1..104: mol_type = other RNA; note = NLB59
ggagctcagccttcactgcagttaccaagtcttgatatactggaactgcttcggcaggttcgtttgcctcccgttcgtcgtcatggcaccacggtcggatccac

SEQ ID NO: 147 - RNA, 103 nt, synthetic construct
  source 1..103: mol_type = other RNA; note = NLB60
ggagctcagccttcactgccacgatggacagtttgaaaactgtttctgcttcggcagggatatctcccttcgtgcgcgcgttaggcaccacggtcggatccac

SEQ ID NO: 148 - RNA, 103 nt, synthetic construct; identical to SEQ ID NO: 66
  source 1..103: mol_type = other RNA; note = NLB61

SEQ ID NO: 149 - RNA, 103 nt, synthetic construct
  source 1..103: mol_type = other RNA; note = NLB62
ggagctcagccttcactgcagaggtgcacttccgccaaggtacttctgcttcggcagtgtgcgatgttctcttgacaaagacaggcaccacggtcggatccac

SEQ ID NO: 150 - RNA, 103 nt, synthetic construct; identical to SEQ ID NO: 60
  source 1..103: mol_type = other RNA; note = NLB63

SEQ ID NO: 151 - RNA, 102 nt, synthetic construct
  source 1..102: mol_type = other RNA; note = NLB65
ggagctcagccttcactgcgttggtatgttactcttgaataagtgctgcttcgcagtgattcgcgacttctgcccctgtctcggcaccacggtcggatccac

SEQ ID NO: 152 - RNA, 104 nt, synthetic construct
  source 1..104: mol_type = other RNA; note = NLB67
ggagctcagccttcactgctcttccgatctcatgcttcagccttcactgctgcggaaatcggcttggcgttgatcatatcctgtggcaccacggtcggatccac

SEQ ID NO: 153 - RNA, 103 nt, synthetic construct; identical to SEQ ID NO: 68
  source 1..103: mol_type = other RNA; note = NLB68

SEQ ID NO: 154 - RNA, 102 nt, synthetic construct
  source 1..102: mol_type = other RNA; note = NLB69
ggagctcagccttcactgccaacagagttacgcttgatgacgtgcctgcttcggcagatgtcttgtggttagcttcatccgtggcaccacggtcggatccac

SEQ ID NO: 155 - RNA, 103 nt, synthetic construct
  source 1..103: mol_type = other RNA; note = NLB70
ggagctcagccttcactgcgtggcttcgttatgacatcgatatatctgcttcggcaggcctaggtgcctttctcgatgcttgtggcaccacggtcggatccac

SEQ ID NO: 156 - RNA, 102 nt, synthetic construct; identical to SEQ ID NO: 82
  source 1..102: mol_type = other RNA; note = NLB71

SEQ ID NO: 157 - RNA, 103 nt, synthetic construct
  source 1..103: mol_type = other RNA; note = NLB73
ggagctcagccttcactgcttccgtgcagtgtagctccctgtcatctgcttcggcagtgccgttgccagtattgcgtcaaactggcaccacggtcggatccac

SEQ ID NO: 158 - RNA, 103 nt, synthetic construct
  source 1..103: mol_type = other RNA; note = NLB76
ggagctcagccttcactgcaaccacgacctccatagtgtgcattcctacttcggcagtcggtgtactaagtcttgacatactaggcaccacggtcggatccac

SEQ ID NO: 159 - RNA, 103 nt, synthetic construct
  source 1..103: mol_type = other RNA; note = NLB79
ggagctcagccttcactgcaggcgagtccgcttgaatacgttcgtctgcttcggcagcgccacttggctcttggtgctgtgggggcaccacggtcggatccac

SEQ ID NO: 160 - RNA, 102 nt, synthetic construct; identical to SEQ ID NO: 73
  source 1..102: mol_type = other RNA; note = NLB80

SEQ ID NO: 161 - RNA, 103 nt, synthetic construct; identical to SEQ ID NO: 64
  source 1..103: mol_type = other RNA; note = NLB81

SEQ ID NO: 162 - RNA, 103 nt, synthetic construct
  source 1..103: mol_type = other RNA; note = NLB83
ggagctcagccttcactgcttgaccgttggtagtttcgagcttcgctgcttcggcagttatcgggttgtgcgctcgcccttgtggcaccacggtcggatccac

SEQ ID NO: 163 - RNA, 103 nt, synthetic construct; identical to SEQ ID NO: 77
  source 1..103: mol_type = other RNA; note = NLB84

SEQ ID NO: 164 - RNA, 102 nt, synthetic construct; identical to SEQ ID NO: 71
  source 1..102: mol_type = other RNA; note = NLB86

SEQ ID NO: 165 - RNA, 103 nt, synthetic construct; identical to SEQ ID NO: 62
  source 1..103: mol_type = other RNA; note = NLB90

SEQ ID NO: 166 - RNA, 103 nt, synthetic construct
  source 1..103: mol_type = other RNA; note = NLB99
ggagctcagccttcactgcagcctcaaatctgcgcaatccgtggtctgcttcggcagtcgcttgaccgtcccgtagtttccggggcaccacggtcggatccac

SEQ ID NO: 167 - RNA, 102 nt, synthetic construct
  source 1..102: mol_type = other RNA; note = NLB100
ggagctcagccttcactgcggtgtaccgtggtccgccgtaatatctgcttcggcagcaagtcttgacatactgcgcctacacggcaccacggtcggatccac

SEQ ID NO: 168 - RNA, 103 nt, synthetic construct
  source 1..103: mol_type = other RNA; note = NLB113
ggagctcagccttcactgcacgcaccgaaagtaagtcttgacatactgcttcggcagaaggcggacagcccagaccgcggcgcggcaccacggtcggatccac

SEQ ID NO: 169 - RNA, 103 nt, synthetic construct
  source 1..103: mol_type = other RNA; note = NLB137
ggagctcagccttcactgctagcgccggattcaagtcttgaattactgtttcggcagctagtgacgtgtggcctgccctaacgggcaccacggtcggatccac

SEQ ID NO: 170 - RNA, 103 nt, synthetic construct; identical to SEQ ID NO: 72
  source 1..103: mol_type = other RNA; note = NLB138

SEQ ID NO: 171 - RNA, 103 nt, synthetic construct; identical to SEQ ID NO: 61
  source 1..103: mol_type = other RNA; note = NLB217

SEQ ID NO: 172 - RNA, 98 nt, synthetic construct
  source 1..98: mol_type = other RNA; note = non-preL
ggagctcagccttcactgctacccccgccagcgtgtcttgacatactgcggcggggtttgatcctcgagacctgtcttggcaccacggtcggatccac

SEQ ID NO: 173 - RNA, 106 nt, synthetic construct
  source 1..106: mol_type = other RNA; note = RNAI
acagtatttggtatctgcgctctgctgaagccagttaccttcggaaaaagagttggtagctcttgatccggcaaacaaaccaccgctggtagcggtggtttttttt

SEQ ID NO: 174 - RNA, 113 nt, synthetic construct
  source 1..113: mol_type = other RNA; note = Ref-RNA
agatcacagagatgtgatggaaaatagttgatgagttgtttaattttaagaatttttatcttaattaaggaaggagtgatttcaatggcacaagatatcatttcaacaatcgg

SEQ ID NO: 175 - RNA, 69 nt, synthetic construct
  source 1..69: mol_type = other RNA; note = RT-F1
ggagctcagccttcactgcacgatagtcgcccatggccacattatacgaggcaccacggtcggatccac

SEQ ID NO: 176 - RNA, 69 nt, synthetic construct
  source 1..69: mol_type = other RNA; note = RT-F2
ggagctcagccttcactgcactattgtgacccctagaacagtggttttcggcaccacggtcggatccac

SEQ ID NO: 177 - RNA, 69 nt, synthetic construct
  source 1..69: mol_type = other RNA; note = RT-F3
ggagctcagccttcactgctgttggagcacttacaaagacgcctagggaggcaccacggtcggatccac

SEQ ID NO: 178 - RNA, 69 nt, synthetic construct
  source 1..69: mol_type = other RNA; note = RT-F4
ggagctcagccttcactgcgaagaaccgttcgcgtctcgctcaatgcgtggcaccacggtcggatccac

SEQ ID NO: 179 - RNA, 69 nt, synthetic construct
  source 1..69: mol_type = other RNA; note = RT-F5
ggagctcagccttcactgcacactttatgtccagggcatctgaaggattggcaccacggtcggatccac

SEQ ID NO: 180 - RNA, 69 nt, synthetic construct
  source 1..69: mol_type = other RNA; note = RT-F6
ggagctcagccttcactgcgtctgtcatgataatgggatcgttacggcgggcaccacggtcggatccac

SEQ ID NO: 181 - RNA, 69 nt, synthetic construct
  source 1..69: mol_type = other RNA; note = RT-F7
ggagctcagccttcactgcggtgagaaatcgtgtactcatctaccgtaaggcaccacggtcggatccac

SEQ ID NO: 182 - RNA, 69 nt, synthetic construct
  source 1..69: mol_type = other RNA; note = RT-F8
ggagctcagccttcactgcccgccttataaagggtagtgacatctgttaggcaccacggtcggatccac

SEQ ID NO: 183 - RNA, 69 nt, synthetic construct
  source 1..69: mol_type = other RNA; note = RT-F9
ggagctcagccttcactgcaacaaccggcctagtgtaggtctggtcatgggcaccacggtcggatccac

SEQ ID NO: 184 - RNA, 69 nt, synthetic construct
  source 1..69: mol_type = other RNA; note = RT-F10
ggagctcagccttcactgctgcgaccggttcagtaagcagaatgggacaggcaccacggtcggatccac

SEQ ID NO: 185 - RNA, 69 nt, synthetic construct
  source 1..69: mol_type = other RNA; note = RT-F11
ggagctcagccttcactgcgaagctatgatcgagcatacgacccagagaggcaccacggtcggatccac

SEQ ID NO: 186 - RNA, 69 nt, synthetic construct
  source 1..69: mol_type = other RNA; note = RT-F12
ggagctcagccttcactgcttaggtactaggctgtagtaagtgctatgaggcaccacggtcggatccac

SEQ ID NO: 187 - RNA, 69 nt, synthetic construct
  source 1..69: mol_type = other RNA; note = RT-Fcg1
ggagctcagccttcactgctggagattccgggttcactattacaaggtaggcaccacggtcggatccac

SEQ ID NO: 188 - RNA, 69 nt, synthetic construct
  source 1..69: mol_type = other RNA; note = RT-Fcg2
ggagctcagccttcactgcgctttcttgtccgtggtaacgggccggcgtggcaccacggtcggatccac

SEQ ID NO: 189 - RNA, 69 nt, synthetic construct
  source 1..69: mol_type = other RNA; note = RT-Fcg3
ggagctcagccttcactgcaggatattctcacgggccattttgatccgtggcaccacggtcggatccac

SEQ ID NO: 190 - RNA, 69 nt, synthetic construct
  source 1..69: mol_type = other RNA; note = RT-Fcg4
ggagctcagccttcactgcaactacgtaacaggacgatccgggctgaatggcaccacggtcggatccac

SEQ ID NO: 191 - RNA, 69 nt, synthetic construct
  source 1..69: mol_type = other RNA; note = RT-FN1
ggagctcagccttcactgctctaatacgactcactataggagctcagccggcaccacggtcggatccac

SEQ ID NO: 192 - RNA, 59 nt, synthetic construct
  source 1..59: mol_type = other RNA; note = RT-FN2(n)
ggagctcagccttcactgctacccccgccagcgtgtcttggcaccacggtcggatccac

SEQ ID NO: 193 - RNA, 69 nt, synthetic construct
  source 1..69: mol_type = other RNA; note = RT-FN3
ggagctcagccttcactgcattctgtaaacaaatggcagcaggtggagtggcaccacggtcggatccac

SEQ ID NO: 194 - RNA, 59 nt, synthetic construct
  source 1..59: mol_type = other RNA; note = RT-FN4(n)
ggagctcagccttcactgctattgacgagatttctcttaggcaccacggtcggatccac

SEQ ID NO: 195 - RNA, 59 nt, synthetic construct
  source 1..59: mol_type = other RNA; note = RT-FN5(n)
ggagctcagccttcactgctggtttatccttcgttacaaggcaccacggtcggatccac

SEQ ID NO: 196 - RNA, 69 nt, synthetic construct
  source 1..69: mol_type = other RNA; note = RT-FN6
ggagctcagccttcactgcgctttttccggggaccgcccaggttgttttggcaccacggtcggatccac

SEQ ID NO: 197 - RNA, 59 nt, synthetic construct
  source 1..59: mol_type = other RNA; note = RT-FN7(n)
ggagctcagccttcactgccggaaccttcggtcagtcacggcaccacggtcggatccac

SEQ ID NO: 198 - RNA, 59 nt, synthetic construct
  source 1..59: mol_type = other RNA; note = RT-FN8(n)
ggagctcagccttcactgcccacggtcaccgtaaaactcggcaccacggtcggatccac

SEQ ID NO: 199 - RNA, 59 nt, synthetic construct
  source 1..59: mol_type = other RNA; note = RT-FN9(n)
ggagctcagccttcactgcgtcaaagatactataccgtcggcaccacggtcggatccac

SEQ ID NO: 200 - RNA, 59 nt, synthetic construct
  source 1..59: mol_type = other RNA; note = RT-FN10(n)
ggagctcagccttcactgcgagatgcaagaacaagcgtaggcaccacggtcggatccac

SEQ ID NO: 201 - RNA, 69 nt, synthetic construct
  source 1..69: mol_type = other RNA; note = RT-FN11
ggagctcagccttcactgctctacggccaaaaagcagataaggttatagggcaccacggtcggatccac

SEQ ID NO: 202 - RNA, 69 nt, synthetic construct
  source 1..69: mol_type = other RNA; note = RT-FN12
ggagctcagccttcactgcaatgcttcccaaccagatcatcgaccttttggcaccacggtcggatccac

SEQ ID NO: 203 - RNA, 69 nt, synthetic construct
  source 1..69: mol_type = other RNA; note = RT-FNcgp1
ggagctcagccttcactgcggtgagggccgcacagttccgggtcctgctggcaccacggtcggatccac

SEQ ID NO: 204 - RNA, 69 nt, synthetic construct
  source 1..69: mol_type = other RNA; note = RT-FNcg2
ggagctcagccttcactgcgagatggtctccgggaatggcttcagtgttggcaccacggtcggatccac

SEQ ID NO: 205 - RNA, 69 nt, synthetic construct
  source 1..69: mol_type = other RNA; note = RT-FNcg3
ggagctcagccttcactgcagttcaatgcaaaaaatccgggactcttagggcaccacggtcggatccac

SEQ ID NO: 206 - RNA, 69 nt, synthetic construct
  source 1..69: mol_type = other RNA; note = RT-FNcg4
ggagctcagccttcactgcaatgataccagcggctccgcgggactatccggcaccacggtcggatccac

SEQ ID NO: 207 - RNA, 59 nt, synthetic construct
  source 1..59: mol_type = other RNA; note = RT-FF1
ggagctcagccttcactgcaccgggagctcagccttcacggcaccacggtcggatccac

SEQ ID NO: 208 - RNA, 59 nt, synthetic construct
  source 1..59: mol_type = other RNA; note = RT-FF2
ggagctcagccttcactgcggcaccacggtcggatccacggcaccacggtcggatccac

SEQ ID NO: 209 - RNA, 69 nt, synthetic construct
  source 1..69: mol_type = other RNA; note = RT-FF3
ggagctcagccttcactgcatatgtgatggttagttatgagtgatctaaggcaccacggtcggatccac

SEQ ID NO: 210 - RNA, 69 nt, synthetic construct
  source 1..69: mol_type = other RNA; note = RT-FF4
ggagctcagccttcactgcattgcaagctaaacgccttgtacaagttcgggcaccacggtcggatccac

SEQ ID NO: 211 - RNA, 69 nt, synthetic construct
  source 1..69: mol_type = other RNA; note = RT-FF5
ggagctcagccttcactgcatagcggcaagaggaacgcaaaggacacctggcaccacggtcggatccac

SEQ ID NO: 212 - RNA, 59 nt, synthetic construct
  source 1..59: mol_type = other RNA; note = RT-FF6
ggagctcagccttcactgcgccggcagcttccgacactaggcaccacggtcggatccac

SEQ ID NO: 213 - RNA, 59 nt, synthetic construct
  source 1..59: mol_type = other RNA; note = RT-FF7
ggagctcagccttcactgctcaagcgcactccttagcacggcaccacggtcggatccac

SEQ ID NO: 214 - RNA, 59 nt, synthetic construct
  source 1..59: mol_type = other RNA; note = RT-FF8
ggagctcagccttcactgcgacttggtattgtatcacaaggcaccacggtcggatccac

SEQ ID NO: 215 - RNA, 69 nt, synthetic construct
  source 1..69: mol_type = other RNA; note = RT-FF9
ggagctcagccttcactgcaatctgggaatgactactagtacttcaagtggcaccacggtcggatccac

SEQ ID NO: 216 - RNA, 59 nt, synthetic construct
  source 1..59: mol_type = other RNA; note = RT-FF10
ggagctcagccttcactgcgcaagggaaacaccgggctgggcaccacggtcggatccac

SEQ ID NO: 217 - RNA, 59 nt, synthetic construct
  source 1..59: mol_type = other RNA; note = RT-FF11
ggagctcagccttcactgcgcgcgctcgaatacgtctatggcaccacggtcggatccac

SEQ ID NO: 218 - RNA, 59 nt, synthetic construct
  source 1..59: mol_type = other RNA; note = RT-FF12
ggagctcagccttcactgccctgcatgtgcaatcactgaggcaccacggtcggatccac

SEQ ID NO: 219 - RNA, 69 nt, synthetic construct
  source 1..69: mol_type = other RNA; note = RT-FFcg1
ggagctcagccttcactgctccgggccgacaatgcgaccgaggtatcacggcaccacggtcggatccac

SEQ ID NO: 220 - RNA, 69 nt, synthetic construct
  source 1..69: mol_type = other RNA; note = RT-FFcg2
ggagctcagccttcactgcggtcgggacctgaccaatccgggagtcagtggcaccacggtcggatccac

SEQ ID NO: 221 - RNA, 69 nt, synthetic construct
  source 1..69: mol_type = other RNA; note = RT-FFcg3
ggagctcagccttcactgcgtacacaacgaacttgctgttccgggtagaggcaccacggtcggatccac

SEQ ID NO: 222 - RNA, 69 nt, synthetic construct
  source 1..69: mol_type = other RNA; note = RT-FFcg4
ggagctcagccttcactgctaacgggtccgtttcaatccttacctgttgggcaccacggtcggatccac

SEQ ID NO: 223 - RNA, 71 nt, synthetic construct
  source 1..71: mol_type = other RNA; note = RT-ubique
ggagctcagccttcactgcgggattcgtgtttatcctcactgaatacgctgggcaccacggtcggatccac

SEQ ID NO: 224 - RNA, 118 nt, synthetic construct
  source 1..118: mol_type = other RNA; note = 70N89
gggaaaagcgaatcatacacaagacccaagagctaacttcccgaaagcagaaatagctgggaggcttttggagcgtcgtggatgcataccgtgcgggcataaggtatttaattccata

(19) United States
(12) Patent Application Publication
(10) Pub. No.: US 2025/0376675 A1
(43) Pub. Date: Dec. 11, 2025

(54) METHOD FOR SCREENING RNA APTAMER

(71) Applicant: Yaqing ZHANG, Zhuzhou, Hunan (CN)

(72) Inventor: Yaqing ZHANG, Zhuzhou, Hunan (CN)

(21) Appl. No.: 18/872,972

(22) PCT Filed: Jun. 8, 2023
(86) PCT No.: PCT/CN2023/099222

§ 371 Date: Dec. 9, 2024

(30) Foreign Application Priority Data
Jun. 8, 2022 (CN) 202210648004.5

(51) Int. Cl. C12N 15/10; C12N 15/115

(52) U.S. Cl. CPC C12N 15/1048; C12N 15/1089; C12N 15/115; C12N 2310/16; C12N 2310/531

(57) ABSTRACT

The present invention provides a method for screening an aptamer from an RNA library. According to the method, an eluent eluted each time is collected and a specific elution program is combined at the same time, thereby not losing any information of the RNA aptamer and achieving the technical effects of low false positive rate, high binding capacity of the screened aptamer, short library preparation time, capability of performing only one round of enrichment, high library preparation repeatability and suitability for an automatic mechanical arm.

### TECHNICAL FIELD

The present invention relates to the field of biotechnology. In particular, the present invention relates to a method for screening RNA aptamers.

### BACKGROUND

Over the past 30 years, technologies, such as the second-generation high-throughput gene sequencing (essentially independent of the screening process, providing only sequence information), microfluidic microarrays (sophisticated equipment and manual adjustment of empirical parameters by specialists), capillary electrophoresis (sophisticated equipment and only suitable for screening for aptamers bound to macromolecules), and bioinformatic modelling of subsequences and structures (data-driven and still dependent on data quality, with a high number of false positives), have been further optimised to shorten the screening process for RNA aptamers, however, there still lack fast, efficient and versatile screening techniques.

In the face of the current global pandemic of Covid-19, developed RNA drug candidates can have the dual identity of vaccine and therapy, with high specificity and safety, less affected by the mutation of Covid-19, short development cycle and low product cost. In addition, in the face of Alzheimer's disease, which is highlighted by the long-term aging population, RNA drug candidates can also have the advantages of dynamic increase/decrease regulation, strong targeting and safety, as well as intelligent precision medicine.

However, existing RNA aptamer screening techniques still have limitations, such as high false positive rate; non-optimal binding ability of the screened aptamer; high time cost, which often requires 10-16 rounds of repetitive screening and 2-6 months of research and development; poor reproducibility; need for manual operation, and other obvious drawbacks, which constrain the screening of RNA aptamers as well as their subsequent applications.

Therefore, there is an urgent need for a new method for screening RNA aptamers to overcome the shortcomings of the prior art.

### SUMMARY OF THE INVENTION

The purpose of the present invention is to provide a method for screening RNA aptamers, which can reduce the false positive rate; optimise the screening conditions and enhance the screening ability; shorten the experiment time and reduce the time cost; improve the screening process and achieve repeatability; and at the same time, can simplify the experimental operation and adapt mechanical intelligence.

In the first aspect, the present invention provides a method for screening RNA aptamers, comprising following steps:
1) providing a library of RNA aptamers to be screened;
2) incubating the library of step 1) with a solid carrier fixed with a target, thereby inducing the RNA aptamer in the library to sufficiently bind to the target;
3) adopting a buffer gradient to elute the RNA aptamers bound to the target on the solid carrier in step 2), and collecting the eluate for each elution, respectively;
4) completely eluting the RNA aptamers still retained on the solid carrier after step 3), and collecting the eluate as the last group of eluate;
5) optionally concentrating and purifying the RNA aptamers in the eluates obtained in steps 3) and 4);
6) reverse-transcribing the RNA aptamers obtained in step 5) to obtain cDNAs;
7) amplifying and high-throughput sequencing the cDNAs obtained in step 6) to obtain sequencing data;
8) analysing the sequencing data obtained in step 7) and sorting RNA aptamer candidate sequences according to their binding potential from high to low, thereby obtaining high-affinity RNA aptamer sequences.

In a preferred embodiment, the providing a RNA aptamer library to be screened comprises preparing the RNA aptamer library in-house, purchasing the RNA aptamer library commercially, or obtaining the RNA aptamer library as a gift from another person.

In a specific embodiment, in step 2), after the RNA aptamer in the RNA aptamer library binds to the target, the solid carrier can be blocked to control and reduce non-specific background binding.

In a specific embodiment, the blocking refers to blocking the solid carrier with a non-target specific random RNA; or blocking the solid carrier with a target specific RNA.

In a preferred embodiment, in step 2), the solid carrier includes, but is not limited to: magnetic beads, matrix.

In a preferred embodiment, the matrix includes, but is not limited to: agarose gel matrix, cephalosporin beads, nitrocellulose, polyvinylidene difluoride membranes, octyl alginate, and other carrier matrices.

In a preferred embodiment, in step 2), the target is a small molecule, including but not limited to: steroids, dopamine, kanamycin, digoxin, antoxin, dinitroaniline, melamine, quinolone, aflatoxin; or a large molecule, including but not limited to: polypeptides, proteins (e.g., enzymes and antibodies, etc.) and complexes (proteins bound with RNA), macromolecules and compounds, and the like.

In a specific embodiment, in step 3), the gradient elution is an elution with a buffer of increased volume, or with a buffer of increased elution strength; preferably an elution with a buffer of increased volume.

In a preferred embodiment, the buffer with increased elution strength is a buffer that prevents the RNA from folding to form a spatial structure by for example, increasing the concentration of salt ions or chelating agents.

In a specific embodiment, prior to the gradient elution, several background elutions are performed until the number of molecules of RNA aptamer contained in the eluate is not greater than 1% of the high throughput sequencing threshold.

In a preferred embodiment, the volume of buffer for background elution should be not greater than the initial volume of buffer used for gradient elution.

In a preferred embodiment, the elution may be a static elution (discontinuous elution, collecting the complete eluate at once) or a dynamic elution (continuous elution, continuously collecting a small amount of partial eluate), preferably a static elution.

In a preferred embodiment, when a static elution is used, the last background elution is performed in a new vessel.

In a preferred embodiment, the volume of the buffer for background elution may or may not be increased, preferably not increased.

In a preferred embodiment, the buffer for background elution and the buffer for gradient elution may be the same or different; preferably the same.

In a preferred embodiment, the buffer for the gradient elution comprises magnesium ions, preferably 5 mM magnesium ions, a pH below 8.5, preferably pH 7-8, and a concentration of NaCl or KCl between 75 mM-200 mM.

In a specific embodiment, after several gradient elutions such that the number of molecules of the RNA aptamer contained in the eluate is suitable for sequencing, and preferably the theoretical minimum of the number of molecules in the library is reduced to less than 10⁵, a complete elution is carried out in step 4), so that the RNA aptamers bound to the target on the solid carrier are completely eluted.

In a preferred embodiment, the buffer for the complete elution contains reagents capable of releasing the RNA aptamer, including reagents capable of disrupting the binding of the target to the solid carrier, and/or reagents capable of disrupting the binding of the RNA aptamer to the target, and/or reagents directly disrupting the target.

In a specific embodiment, in step 7), a compensating sequence of 0-6 nt is randomly inserted between a sequencing linker and a cDNA constant region.

In a specific embodiment, in step 7), a custom-designed PhiX is introduced to further compensate the unbalanced base distribution in the constant region during the mixing of the multiple samples.

In a specific embodiment, in step 8), the binding potential means that the degree of enrichment increases fast in each eluate, rather than only considering the highest degree of enrichment.

In a specific embodiment, the binding potential is judged according to one or more of the following information about the RNA aptamer: the abundance of the RNA aptamer in each eluate, the number of times the RNA aptamer has been detected individually in each eluate, and the preference of the RNA aptamer to be present in subsequent eluates over the initial eluate.

In a preferred embodiment, the above information is combined to fit a standard curve to judge the binding potential of the RNA aptamer according to the area under the curve (AUC).

In a preferred embodiment, the RNA aptamer comprises a chemically modified sequence.

In a preferred embodiment, the chemically modified sequence is a fluorine-modified sequence.

In the second aspect, the present invention provides an RNA aptamer, which is screened and obtained by using the method of the first aspect.

In a preferred embodiment, the RNA aptamer comprises a RNA aptamer with known sequence and random modifications on different bases (e.g. A, U, G, C).

In a preferred embodiment, the RNA aptamer does not comprise a conventional RNA aptamer with known sequence and no additional modifications.

In a preferred embodiment, the RNA aptamer comprises a chemically modified sequence; preferably a fluorine modified sequence.

In the third aspect, the present invention provides an apparatus for performing the method of the first aspect above.

In a preferred embodiment, the apparatus comprises following modules:
1) a preparation module for preparing a library of RNA aptamers to be screened;
2) an incubating module for incudating the prepared library with a solid carrier (magnetic beads or matrix) fixed with a target;
3) an elution and collection module for performing a gradient elution to elute the RNA aptamers bound to the target on the solid carrier, and collecting the eluate for each elution, respectively;
4) an optional concentration and purification module for concentrating and purifying the RNA aptamer in the eluates;
5) a reverse-transcription module for reverse-transcribing the RNA aptamers to obtain CDNAs;
6) an amplification and high-throughput sequencing module for amplifying and high-throughput sequencing the cDNAs obtained above to obtain sequencing data; and
7) an analysis module for analysing said sequencing data and sorting the RNA aptamer candidate sequences according to their binding potential from high to low, thereby obtaining RNA aptamer sequences with high binding affinity.

In the fourth aspect, the present invention provides a biochip comprising the RNA aptamers of the second aspect.

In the fifth aspect, the present invention provides a method for preparing a biochip, comprising steps of:
1) screening and obtaining RNA aptamers using the method of the first aspect; and
2) preparing a biochip using the RNA aptamers screened and obtained in step 1).

In the sixth aspect, the present invention provides a pharmaceutical composition comprising the RNA aptamers of the second aspect and a pharmaceutically acceptable excipient or drug delivery carrier.

In the seventh aspect, the present invention provides a drug delivery carrier, which is attached to the RNA aptamers of the second aspect.

In a preferred embodiment, the drug delivery carrier is a liposome.

In a specific embodiment, where there are specific recognition receptors on the surface of a cell, the RNA aptamer of the present invention can be glued or attached to a delivery carrier (e.g. a nanoliposome), thereby enabling a specific delivery of a drug encapsulated within the carrier to a designated cell.

In the eighth aspect, the present invention provides a diagnostic reagent comprising the RNA aptamers of the second aspect and other auxiliary reagents required for the diagnosis.

In the ninth aspect, the present invention provides the use of the RNA aptamers screened and obtained by using the method of the first aspect for preparing a biochip, a pharmaceutical composition or a diagnostic reagent.

It should be understood that each of the above technical features of the present invention and each of the technical features specifically described below (e.g., in the Examples) can be combined with each other within the scope of the present invention, thereby constituting new or preferred technical solutions, which will not be repeated one by one herein due to the limited contents.

### DESCRIPTION OF DRAWINGS

FIG. 1 shows the construction and modelling analysis of an RNA library of the present invention. “a” shows the screening RNA and sequencing library construction of the present invention. Firstly, a random RNA library source is incubated with targeting molecules ligated with magnetic beads. The mixture of targeting molecules bound with RNAs is sequentially washed with the same binding buffer in increased volume gradients and the eluates are collected, named as groups 1-10 (g1-10). Then, a final elution is performed on the mixture (group 11, g11) with an appropriate chemical or enzyme to completely detach RNAs strongly bound to the target from the magnetic beads. The purified RNAs from g1-11 are subjected to reverse transcription, offset PCR amplification and Illumina PCR amplification, and finally customized PhiX is added to balance the library base distribution before sequencing. “b” shows the sequencing data of the present invention for mining high affinity aptamers. Sequencing raw sequences are pre-treated into a 67 nt core region. The sequence after data cleaning is counted and merged into the same data frame. The sequences on each non-initial group (g2-11 relative to g1) are normalized based on the default initial gamma baseline and the multiplicative ratio weights are adjusted. For sequences in each change rate group, their gamma change rate (gf) is represented by the area under the curve (auc). Based on the sub-sequence characteristics of the top-ranked sequences and their enrichment routes on each group, the ranking model can be further fine-tuned. Finally, the aptamer sequences with the highest values of auc are selected for downstream functional applications.

FIG. 2 shows the construction features of the RNA library of the present invention. “a” is the sequence composition of the random RNA library source of the present invention. From the 5′-end to the 3′-end, the RNA sequence (103 nt) contains the primer A binding region (19 nt, purple square, sequence labelled below), the left arm random region (N26, 26 nt), the pre-formed loop (L12, 12nt) region, the right arm random region (N26, 26 nt), and the primer B binding region (20 nt, dark green square, sequence labelled below). “b” is the reverse-transcribed single-stranded cDNA template sequence used for offset PCR amplification. The dark blue and purple shaded sequences represent the partial binding regions with primer B and primer A, respectively. “c” is the sequence composition of the dsDNA library of the present invention used for sequencing. RNA sequences consisting of a pre-formed loop (L12) region of a fixed sequence and two random 26 nt (N26) regions are reverse complementary to the dsDNA. “d” is a combination of two versions of the offsetPCR multiplex primers. The forward primer (‘Frw’) comprises the sequencing 5′-end adaptor sequence (shaded grey), 0-6 nt compensating sequence (orange letters), and a portion of the primer B region sequence (dark green letters), while the reverse primer ('Rev') comprises the sequencing 3′-end adaptor sequence (shaded green), 0-6 nt compensating sequence (orange letters), and a portion of the primer A region sequence (purple letters). This designed version 1 (V1) compensating primer is 2 nt longer than the version 2 (V2) compensating primer. “e” is a customized PhiX sequence specifically designed for the present invention to balance the uneven distribution of bases. The customised PhiX includes sequencing adaptor sequences at the 5′ and 3′ ends, with random nucleotides (denoted by ‘N’). Nucleotides shaded in light blue are used to compensate for base bias. “F” is an electrophoretic analysis of offset PCR and Illuminal PCR products by Bioanlyzerd. x-axis indicates the length of the dsDNA, while the y-axis corresponds to the fluorophore signal intensity. The name abbreviations “V1” and “V2” are the same as in the “d” panel, while “52.5C”, “51C”, and “68.5C” represent PCR annealing temperatures. “g” is a distribution plot of compensating sequences in the sequenced library. x-axis represents the length of compensating sequences identified from the raw reads, while the y-axis represents the percentage of compensating sequences in the library with the indicated lengths. The torch shaped box plot consists of compensating sequences with specified lengths from the 11 groups (n=11) of the present invention. “RLA”, “RLB”, and “RLC” are the abbreviated names of the “+None”, “tRNA”, and “cRNA” background closure systems applied to in silico rhodopsin in the RNA libraries of the present invention. Similarly, “NLA”, “NLB” and “NLC” are abbreviations for the corresponding background closure systems applied to the Covid-19 replicase nsp12. The dashed line represents the percentage of the ideal compensating sequence with an average distribution (14.28 percent, 100/7). “h” is a plot of the percentage distribution of the library sequences after processing of the corresponding data. The x-axis represents the process from the number of original sequences (“raw”) to the trimming of compensating sequences (“after_offset”), to the establishment of pre-formed loops (“after-bridge”), to the processing of unknown sequence signals (“after-N”), while the y-axis represents the percentage of sequences that pass through this process. The torch shaped box plot is composed of the percentage of sequences from the 11 groups (n=11) of the present invention that meet the selection criteria in the corresponding processing steps. The abbreviation of the name is the same as that in Figure f. The dashed line represents a percentage of 94%.

FIG. 3 shows a comparison of the gradient reconstruction (SGRELI) ability of the enriched ligand system between the present invention and the prior art. “a” represents the enrichment abundance trend of three representative high affinity aptamers in each sub library (group (g)/round (r)) in the present invention and the prior art. RLA, RLB, and RLC are abbreviated names for the library of the present invention (similar to FIG. 2g), while RC is an abbreviated name for the conventional library. The lines of different colors represent the enrichment trends of different aptamers, and the dots on each line represent the abundance of the corresponding aptamer on the specified sub library. The arrow represents the abundance trend from the second/third to last sub library to the last sub library. The x-axis represents the order of sub libraries, while the y-axis represents the relative sequence abundance (per million qualified sequences (RPM)). “b” is the average pearson similarity coefficient between the sub sequences of the enriched sequences from the present invention/prior art sub library and the corresponding sub sequences from the Dope directed random library validation dataset. The abbreviated names, arrows, and x-axis of the sub library are the same as those in FIG. 2a. The y-axis represents the average pearson correlation coefficient value, and the gray dashed line represents the reference value of 0.6. The solid lines of different colors represent the number of sequences with different enrichment levels (“t1k” represents the top 1000, “t10k” represents the top 10000, “t100k” represents the top 100000, and “all” represents all sequences) for the calculation of subsequences, while the points in the lines represent the average pearson correlation coefficient of each validation data calculated using the specified number of enrichment sequences and the specified sub eluent group. The first line of analysis is based on a subsequence length of 6 nt (“n6” in the legend), while the second line uses 10 nt (“n10” in the legend).

FIG. 4 shows the maximum threshold and trend characteristics of SGRELI in the present invention. “a” shows the abundance correlation of sub features (6 nt) of the first 10000 sequence enriched in the sub libraries of the present invention/prior art and the Dope directed random validation dataset. The x-axis represents the relative abundance of log 10 logarithmic transformation of the sub features of the present invention/conventional technology, while the y-axis represents the relative abundance of the Dope validation dataset. The blue dots represent the correlations calculated based on the seq A directed validation library, while the red and green dots represent the correlations based on the seq B and seq C directed random libraries, respectively. The legend provides pearson correlation coefficients for all points between the present invention/conventional technology and the corresponding doping library source (“pA” represents the directed seq A library, “pB” and “pC” represent the directed random seq B and seq C libraries, respectively). The abbreviated names of the sub libraries (“gx” and “rx”) and libraries (“RLX” and “RC”) are the same as those in FIG. 3a. “b” shows the same experiment and analysis as FIG. 3b, however, 5 nt, 7 nt, 8 nt, and 9 nt are used as the length of the subsequences for correlation analysis. “C” shows the average pearson correlation coefficients of subsequences of different lengths in the first 10000 sequences of the sub library of the present invention/prior art and Dope directed random validation dataset. The abbreviated names of sub libraries (“gx” and “rx”), libraries (“RLX” and “RC”), x-axis, y-axis, and arrows are the same as those in FIG. 3b. The lines of different colors represent subsequences calculated based on the corresponding library source, while the points in the lines represent the average pearson correlation coefficient between the specified sub library and each validation library. The “x-gram” indicates that the x-nt length of the subsequence is applied to pearson correlation calculation. “D” shows the gamma baseline library. The lines (gamma>=1) and dashed lines (gamma<1) of different colors represent the weighted baseline for each fold comparison (referring to Group 1). The y-axis represents the expected enrichment weighted weights.

FIG. 5 shows the fluorescence imaging application in cells of the high affinity silicon rhodamine RNA aptamer selected by the present invention. “a” shows the overlapping intersection of the top 25 RNA aptamers in three libraries of silicon rhodamine (SiR). The percentage represents the proportion of RNA aptamers that bind and activate fluorescent silicon rhodamine (turn on). “b” shows the percentage of overlapping intersection between the SiR three libraries based on the number of aptamers ranked higher in different orders. The x-axis represents the number of RNA aptamers selected for each library during the analysis process, while the y-axis represents the percentage. The green line represents the percentage of RNA aptamers present in all three libraries, while orange and blue represent the percentage of RNA aptamers present in only two libraries and a single library, respectively. “c” shows the KD curves of the high affinity aptamers RLB2 and RLB15, which are ranked higher. 50 nM of SiR-PEG2-NH2 probe is incubated with RNA aptamers of different concentrations. The y-axis represents the measured relative fluorescence intensity. “d” displays the fluorescence intensity multiples of the top 25 RNA aptamers in the RLB library. The X-axis represents the top ranked “N” RNA aptamers (sorted in descending order, red bar), pure dye reference (blue bar), and pure buffer (gray bar). The height of the column represents the fold change in fluorescence intensity, with the signal intensity change of pure dye measured as unit 1 (indicated by the dashed line). The error line is the mean +standard deviation. “e” shows that the library of the present invention has higher fluorescence fold activation than the RNA aptamers ranked higher in the prior art library. The relative fluorescence fold changes of the top ranked aptamers of the present invention from RLA (blue, n=25), RLB (orange, n=25), and RLC (green, n=25) with those from conventional RC (red, n=42) are compared. “f” shows the sorted distribution of RNA aptamers with SiR on and off in the library of the present invention. The y-axis displays the sorted distribution of RNA aptamers in the corresponding library. The purple box (n=20) and gray box (n=21) represent the RNA aptamer groups that are turned on and off, respectively. “g” showed that RLB aptamers have higher fluorescence quantum yields than SiRA, making them more suitable for RNA molecular imaging in live HEK237T cells. Under the action of 200 nM of SiR-PEG-NH2 dye, the target intracellular RNA is displayed in red, and the nuclear region is displayed in blue by Hoechst dye.

FIG. 6 shows the characteristic details of RNA silicon rhodamine aptamers screened according to the present invention. “a” shows that SiR with high gamma values exhibits relatively high screening accuracy. The F1 score is calculated by evaluating the top ranked sequences (predicted “bound”) and the rest (predicted “not bound”) in the library of the present invention, referencing the top ranked sequences (actual “bound”) and the rest (actual “not bound”) in the directed random Dope validation library. The highest F1 (used to adjust enrichment trends) and the percentage of the scale sequence (used to determine the reweighted position) within the baseline boundary value range (0-0.25) for separating true “bound” and true “unbound” buckets are plotted on a heatmap. The maximum F1 score for all given combinations is represented by a white box and numbered in the heatmap, while the F1 score based on gamma 1.0 is represented in red. “b” shows the details of the impact of changing gamma values on SiR sorting accuracy based on a fixed percentage of the ruler sequence (0.01) within the sorting baseline boundary range. The top row represents the effect of the entire baseline range, while the bottom row represents the most valuable high ranking baseline range (0-0.02). Different gamma values are represented in different colors and line formats in the graph. The Sankey panelt in “c” shows the enrichment pathway of SiR aptamer with the highest AUC. The colored bucket “A” represents the top 0.01% sequence in the specified sub library, “B” represents the top 0.01-0.05%, “C” represents the top 0.05-0.1%, “D” represents the top 0.1-0.5%, “E” represents the top 0.5-1%, “F” represents the top 1-5%, “G” represents the top 5-10%, “H” represents the top 10-50%, “I” represents the top 50-100%, and “J” represents not present in the current sub library. The size of the bucket is linearly correlated with the number of sequences within the corresponding highest ratio range, and the flow size is also linearly correlated with the number of sequences between nodes. “d” shows the distribution of AUC values after gamma correction for SiR. In order to perform cross comparison between three SiR libraries, the maximum AUC value was scaled to 1.0. The height of the column represents the number of sequences within the specified AUC range. “e” shows an increasing trend in the normalized ranking factor values of three high affinity SiR aptamers. The x-axis represents the described subclasses of gamma corrected ratio changes (gf), which are the same as those in FIG. 1a. The colored lines represent the enrichment trend of the corresponding library, and the points on the lines represent the sorting factor values of the specified subclasses. The specific numerical values of AUC are used in the legend.

FIG. 7 shows the high affinity COVID-19 replicase RNA aptamer screened by the invention and its application in inhibiting RNA replication. “a” shows the overlapping intersection of the top 25 RNA aptamers in three libraries of COVID-19 replicase (nsp12). The percentage in the figure represents the proportion of high affinity RNA aptamers. “b” shows the same experimental and analytical process as FIG. 5b, but using the nsp 12 library as the data source. “c” shows the binding details between the RNA aptamer of the present invention and nsp 12 protein characterized using biofilm interference (BLI). The binding process (left) and dissociation process (right) are separated by a vertical broken line. The curves of different colors represent the use of corresponding RNA concentrations as measurement conditions. “d” shows the KD values of 77 randomly selected NLB aptamers of the present invention and their statistical ranking in the library. The horizontal and vertical histograms represent the distribution of library sorting and KD values, respectively. The dark green dots (n=17) indicate that KD is based on three sets of independently repeated multiple measurement data, while the light green dots (n=60) are based on two sets of independently repeated single measurement data. The numbers near the dots indicate their sorting in the library. The horizontal dashed line represents a KD of 10 nM, while the gray percentage values indicate the proportion of aptamers above and below this boundary. “e” shows the sorting distribution of high affinity RNA aptamers in libraries of the present invention and traditional libraries in FIG. 7d. NCA (n=68), NCB (n=66), and NCC (n=66) are libraries screened for nsp12 using traditional methods for 11 rounds, and the blocking systems are the same as those of the NLA (n=77), NLB (n=77), and NLC (n=77) libraries of the present invention. “f” and “g” demonstrate that the addition of the RNA aptamer of the present invention with 3′ end being blocked to the nsp12/7/8 (RdRp) replicase complex species can effectively inhibit elongation. The concentration ratio of RNA template: aptamers: nsp12/7/8 is 2.5 μM: 1.25 μM: 1.25 μM. RNA was separated in denaturing PAGE electrophoresis and visualized (Figure g). The corresponding statistical values of three independent repeated experiments are shown on figure f. The error line represents the mean±standard deviation. “h” and “I” demonstrate the visualization (Figure h) and quantification (Figure i) of the reaction kinetics of the inhibitory effect of the 3′-end blocked aptamers of the present invention. The error bar represents the mean±standard deviation.

FIG. 8 shows the feature details of the application of the screened RNA aptamers of the invention to the COVID-19 replicase nsp 12 and the inhibition of 3′-end modification on polymerase. “a” shows that nsp12 with small gamma values has relatively high screening accuracy. The analysis process is the same as FIG. 6a, however the nsp12 library is used for evaluation, and the last round sequence abundance of the nsp 12 traditional library is used as a reference for validation. The analysis process in “b” is the same as that in FIG. 6b, however the nsp 12 library is used for evaluation, and the last round sequence abundance of the nsp 12 traditional library is used as a reference for validation. The baseline boundary range for analysis is 0-0.2. The analysis program in “c” is the same as that in FIG. 6c, however, the nsp12 library is used for evaluation. The colored bucket “A” represents the sequence of the top 0.00001% of the specified sub library level, “B” represents the top 0.0001-0.0005%, “C” represents the top 0.0005-0.001%, “D” represents the top 0.001-0.005%, “E” represents the top 0.005-0.001%, “F” represents the top 0.01-0.1%, “G” represents the top 0.1-1%, “H” represents the top 1-10%, “I” represents the top 10-100%, and “J” represents not present in the current sub library. The analysis program in “d” is the same as that in FIG. 6d, however the nsp12 library is used for comparison. The analysis program in “e” is the same as that in FIG. 6e, however the nsp 12 RNA aptamers are used. In “f”, a lower concentration of RNA aptamer is used for BLI KD detection, consistent with a higher concentration. The same experimental and analytical process as that in FIG. 5c is used, however a lower RNA concentration is used for measurement. “g” shows the low background signal of RNA aptamers in BLI KD assay. The same experimental and analytical procedure as that in FIG. 5c is used, however a system without added proteins (left figure) or with randomly selected RNAI is used as a reference (right figure). In “h”, the incubation of the RNA aptamers of the present invention of the original nsp12 with RdRp results in observable elongation inhibitory effects. The concentration ratio of RNA template:aptamers:nsp12/7/8 is 2.5 μM:5 μM:2.5 μM. The isolation and visualization of RNA are the same as those in FIG. 7g. In “I”, blocking the 3′-end of the RNA aptamer of the present invention increases the inhibitory effect on RdRp elongation activity. The proportion of inducers. In the inhibition assay, the ratio of RNA template (2.5 μM) to the concentration of the aptamer after the addition of the original (“o”) or blocking treatment (“b”) is used at 2, 1, 0.5, 0.25, and 0.125, respectively. The concentration of nsp 12/7/8 is 2.5 μM. The isolation and visualization of RNA are the same as those in FIG. 7g.

FIG. 9 shows the inhibitory effect of the chemically fluorinated RNA aptamer (pentasaccharide 2′-terminal) screened by the invention on the reverse transcriptase of type I AIDS (HIV-1) in vitro. “a” shows the inhibitory effect of RNA aptamers obtained from three different screening libraries added to the HIV-1 replicase system on replication. The library contains RT-F (“RTF”, RNA containing 30 random sequences (“30F”), single library, fluorinated modified C and U), library RT-FF (“RT-FF”, RNA containing 30 random sequences (“30F”) or containing 20 random sequences (“20F”), mixed library, perfluorinated modified C and U), library RT-FN (“30FN”, template containing 30 random sequences (“30F”), and library containing 20 random sequences (“20F”), mixed library, only 3OF library RNA uses fluorinated modified C and U, while the 20F library does not have fluorination modification). “ubique” is a highly repetitive RNA aptamer in the library, while reference sequence 70N89 is a published nucleic acid aptamer that inhibits HIV-1 replicase. In the measurement system, DNA 5 end Cy3 labeling is used for instrument measurement and visualization. In the reaction system, the concentration ratio of DNA template:DNA primer:RNA adapter:HIV replication enzyme (p66) is 100 nM:100 nM:10 nM:60 nM. In Figure b, the inhibition percentage of RNA aptamer on replication is a visualization of Figure a. The ratio is calculated as the percentage of non extended band brightness to the total brightness of extended and non extended bands, and then normalized to positive control (“PC”) and negative control (“-Enzyme”) for three independent repeated experiments.

### MODES FOR CARRYING OUT THE INVENTION

The inventor conducted extensive and in-depth research on the screening method of RNA aptamers. During the exploration process, it was found that collecting the eluate from each elution will not lose any information about RNA aptamers, and thus combine all “background” washing information to determine the systematic gradient reproducibility (SGRELI) of enriched ligands. The method of the present invention can achieve low false positive rates, strong binding ability of the screened aptamers, short library preparation time, the ability to perform only one round of enrichment, high reproducibility of library preparation, and suitability for automated robotic arms and other technical effects. On this basis, the present invention has been completed.

### RNA Aptamers and the Screening Method Thereof

The terms “RNA aptamer” and “RNA ligand” used herein have the same meaning, referring to a RNA substance that can interact with various biological and chemical targets to regulate their functions. Similar to antibodies, artificially screened short single stranded RNA aptamers specifically recognize and bind to targets by folding into specific three-dimensional structures.

The commonly used aptamer screening techniques in this field mainly involve iterative repeated screening and enrichment of RNA aptamers in RNA libraries. This technology has promoted the widespread application of RNA aptamers, such as live cell super-resolution RNA imaging, SARS-COV-2 RNA detection, and spike protein blockade, and the like. However, it is generally necessary for researchers to conduct 8-16 rounds of repeated screening, followed by extensive Sanger sequencing, which can take several weeks to several months. Although reverse selection, second-generation high-throughput sequencing (NGS), capillary electrophoresis, and microfluidic chip separation have reduced the number of iterations in RNA screening and improved binding specificity, there is still a lack of a rapid screening method for high affinity RNA aptamers.

The inventor developed a rapid screening method for high affinity RNA aptamers and applied it to fluorescent silicon rhodamine (0.6 kDa) for live cell RNA imaging; and also applied to SARS-COV-2 polymerase nsp12 (˜110 kDa) to inhibit the replication of RNA dependent RNA polymerase (RdRp). After a RNA aptamer wet experiment screening for 5 hours, NGS libraries of 11 gradient eluted RNA solutions were established within one day, and sequencing and mathematical modeling sorting analysis were completed. Unlike other methods that rely on the abundance of RNA aptamers in the final enriched eluate to determine high affinity selection, the method of the present invention combines all “background” elution information to determine the systematic gradient reproducibility (SGRELI) of enriched ligands. The RNA aptamers screened by the method of the present invention are high affinity, effective, and reproducible aptamers. The activation of fluorescence by silicon rhodamine and the activity of SARS-COV-2 RNA polymerase have confirmed that the RNA aptamers screened by the method of the present invention can be successfully applied to the functional regulation of targets.

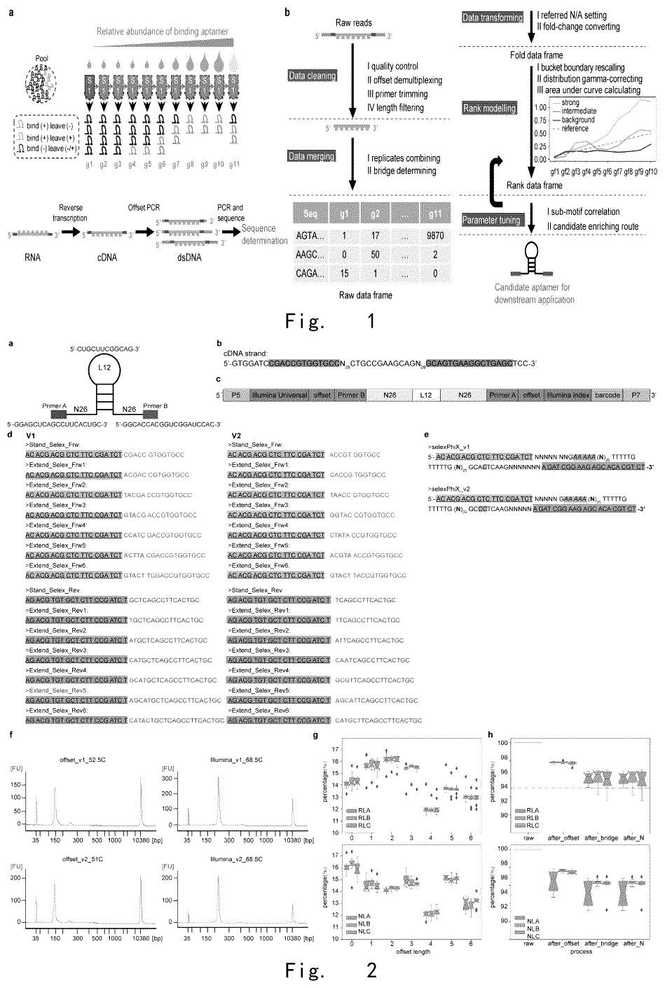

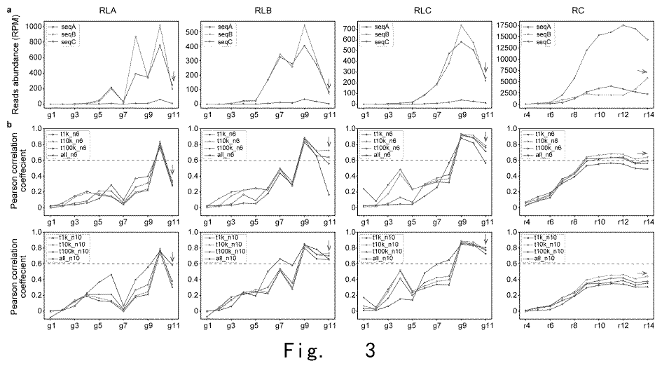

Specifically, the method of the present invention combines wet and dry experiments. The end-to-end method of the present invention consists of two parts: wet experiment and dry experiment (FIG. 1a, b). In order to ensure sufficient diversity of the library, ˜2×10¹⁴ random RNA molecules (FIG. 2a) were used for ligand screening during the experiment. The target molecules labeled with biotin or His were captured by magnetic beads of streptomycin and Ni-NTA, respectively. In order to systematically evaluate the non-specific binding of the method of the present invention, a parallel comparison of three magnetic bead blocking systems: type A (no blocking), type B (blocked with tRNA), and type C (blocked with RNA with known binding ability) was conducted. Within 1 hour, RNAs with different binding and dissociation abilities were harvested from 11 groups by increasing washing pressure (FIG. 1a). The RNA selected from each group were reverse-transcribed into single stranded cDNAs (FIG. 2b).

Due to the presence of constant region sequence in this cDNA at the 5′-/3′-end and the pre-formed loop region in the middle, it is mainly used for PCR primer binding. However, each sequencing cluster produces the same fluorescence signal at the same base position, which can lead to extensive single fluorescence overexposure in Illumina high-throughput sequencing, thereby covering other signals. To address the issue of base imbalance, offset PCR was designed herein, which randomly inserts 0-6 nt compensation sequences between the sequencing linker and the constant region of cDNA (FIG. 2c). Therefore, based on 7 different lengths of cDNA bases, the sequences were correspondingly translated and the base composition was balanced (FIGS. 2d and 2f). Then, the balanced dsDNAs were ligated with sequencing primers and sample labels through PCR. In the process of mixing multiple samples, custom designed PhiX was also introduced to further compensate for the unbalanced base distribution in the constant region (FIGS. 2e and 2g).

When sequencing is completed and the dry experiment section starts, the original sequences of each group are cleaned and merged to obtain a high-quality data framework (FIG. 2h). Subsequently, data conversion, sorting modeling, and parameter adjustment were performed. Finally, the RNA aptamer candidate sequences were sorted from high to low based on their binding potential, thereby completing the entire analysis process in approximately 30 minutes (FIG. 1b).

Compared with conventional methods, SGRELI makes the method of the present invention more advantageous. Conventional procedures will wash away non-specific binding ligands for multiple times, and then collect the final eluate as the collection of the strongest binding ligands. Unlike conventional procedures, the method of the present invention collects information from all eluates, which are typically discarded and ignored in conventional procedures. In order to compare the enrichment performance of the method of the present invention and conventional methods for high affinity RNA inducers, seven SiR-based deep sequencing libraries were generated herein: one derived from a conventional library (RC) from previous work, three libraries of the present invention using magnetic bead closure systems of type A (RLA), type B (RLB), and type C (RLC), and three directionally randomly generated validation libraries based on known SiR-binding aptamer seqA, seqB, and seqC sequences (Table 1).

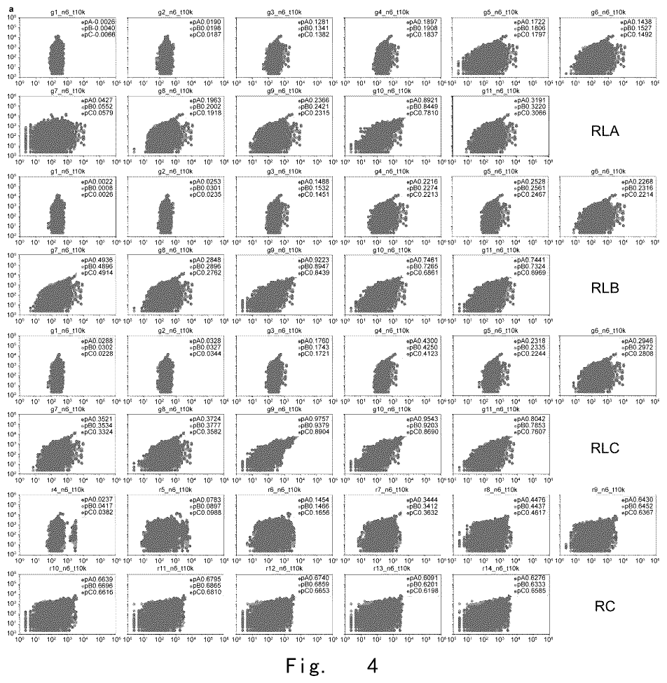

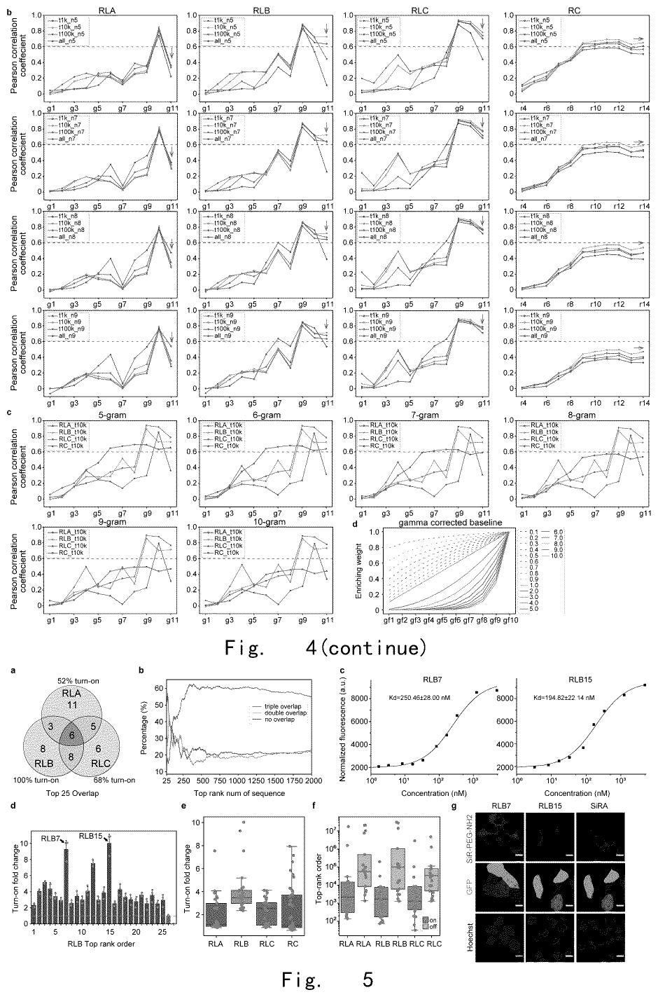

In the 11 groups of the single round method of the present invention, the enrichment trends of the aptamer seqA (KD 430 nM) and seqC (KD 1456 nM) were similar to those of the last 11 rounds of the conventional method, however seqB (KD 25 nM) was specifically discovered in the method of the present invention (FIG. 3a). This indicates that the method of the present invention is effective for the high affinity of RNA aptamers. Consistently, in the last few eluate groups of the method of the present invention, the enrichment abundance of all three aptamers first increases and then decreases, however theoretically, the last group should have the strongest enrichment effect. This sudden decrease indicates that the RNA ligands harvested from the elution step may not be the most ideal enriched ligand group, and may be mixed with more background. In order to eliminate the bias caused by small sample size analysis, the inventor used a validation library as a reference to compare the top-level richness sub features of the sequence between the method of the present invention and the conventional method. As the number of selection groups or rounds increases, the similarity between the sequences with the highest enrichment in the method of the present invention and conventional methods gradually increases (FIG. 3b and FIG. 4a). It is worth noting that the peak of sub feature similarity in the method of the present invention occurs before the final elution step and is higher than the library of conventional methods, regardless of the length of the sub features (FIG. 3b and FIG. 4c). All other analyses of the similarity of subsequence groups of different lengths also found a sudden decrease in similarity in the final elution step of the method of the present invention (FIG. 4b). Therefore, gamma baseline is used herein to simulate the enrichment trend of the method of the present invention (FIG. 4d). By combining these suddenly decreasing features with higher sub feature similarity proofs, the method of the present invention repeatedly and gradiently observed enriched aptamers from the eluate solutions of conventional methods, and the enriched aptamers have lower background signals.

The inventor studied the effect of the RNA silicon rhodamine aptamer obtained by the method of the present invention on activating dye fluorescence signals.

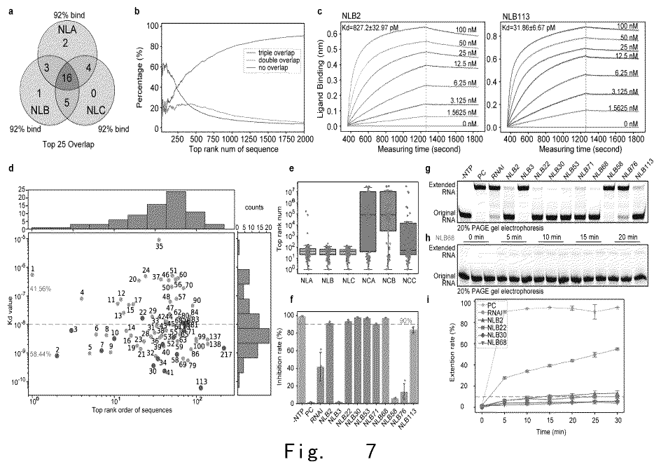

In order to investigate the performance of enriching high-quality aptamers, the SGRELI ranking model was analyzed herein by using 380 hyperparameter gamma values and ruler quantile combinations in three SiR libraries of the present invention. Using the validation library as a reference, the ranking order of these sequences in the present invention library and the conventional library was compared. According to the best f1 score RLC (61)>RLB (59)≥RLA (51)>RC (36), the model with a moderate gamma value (4) produced higher accuracy in predicting the ranking order (FIG. 6a). In addition, the impact of ruler quantile on the accuracy of prediction is relatively small. In order to eliminate the bias caused by different sorting ranges in the validation library, the inventor further analyzed the prediction accuracy of hundreds of different buckets, ranging from 0.00025 to 0.25 in abundance ranking. Similarly, in the top ranked small range, the accuracy of the predicted rank order is the same as the global best f1 score order (FIG. 6b). In order to optimize generalization ability, we analyzed the library using the default gamma value (1.0), which performed well in both the present method and conventional methods. The enrichment routes of the top ranked sequences in the library of the method of the present invention were compared, and the first group that completely covers the top ranked sequences is the RLA library, followed by the RLB and RLC libraries (FIG. 6c). This also conforms to the order in which the system binds to competitors, i.e., no blocking<tRNAs blocking<blocking with RNAs which are known to bind. In addition, it is noted that the buckets of predicted top ranked sequences in the RLB library are much denser than those in RLA and RLC (FIG. 6d). Meanwhile, the RLB library provides a larger AUC score for their ranking (FIG. 6e). This means that RLB libraries may have better enrichment capabilities.

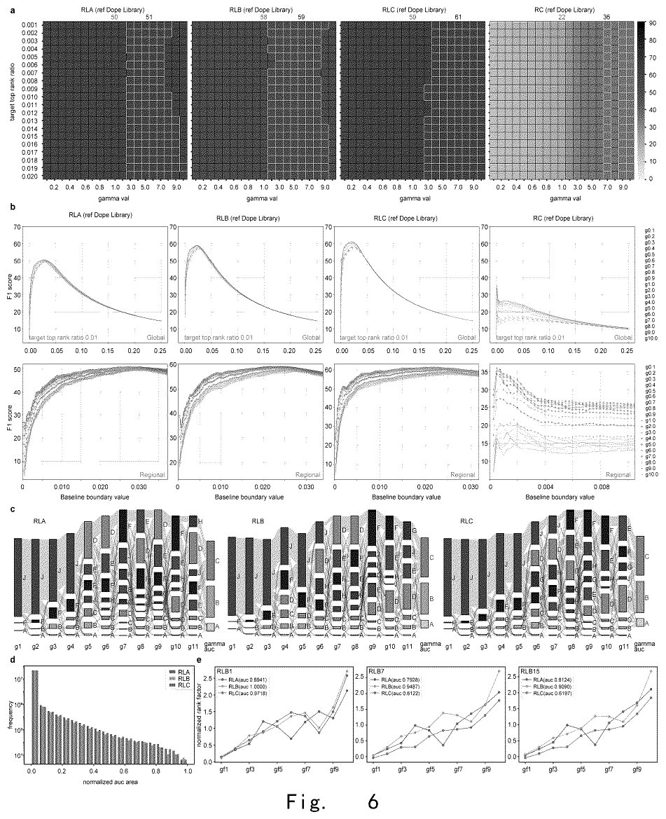

Due to limited manpower, only a small portion of adapters are usually selected for downstream validation, therefore, it is important to understand the performance of the top ranked aptamers in all three libraries of the present invention. Herein, it was found that 100% of the top 25 aptamers of RLB bind to SiR and activate fluorescence, while RLA and RLB are 52% and 68%, respectively (FIG. 3a). Most of the RLB aptamers were also found in RLA and RLC (FIG. 5b), with similar ranking order (FIG. 5f). Although aptamers can also bind to SiR without activating dye fluorescence, since the main goal of such application is to screen for aptamers that can activate fluorescence, aptamers that do not activate dye fluorescence will not be analyzed separately. The two most promising aptamers, RLB7 and RLB15, have a KD about 2 times lower than the reported best aptamer (SiRA, KD=430±70 nM, ˜7-fold fluorescence activation) (FIG. 5c), and a fluorescence rate increase of up to 20% (FIG. 5d). Based on the comparison of the performance of aptamers for activating fluorescence, RLB is the most abundant aptamers library for activating fluorescence signals, followed by RLC, RLA, and RC (FIG. 5e). In addition, aptamers with opening ability rank higher than those without opening ability. Since SiRA ranks around 1000 in all libraries of the present invention, the RLB aptamers in the top 1000 may contain more applicable aptamers. Herein, RLB aptamers were further utilized for live cell RNA imaging, displaying a higher and cleaner signal than SiRA (FIG. 5g). In summary, the method x of the present invention establishes high-quality aptamers and activates SiR for application in live cell RNA imaging.

The inventor also verified the ability of the RNA COVID-19 replicase aptamers obtained by the method of the invention to inhibit the enzyme activity.

Due to the respiratory disease pandemic caused by SARS-COV-2, there is an urgent need to develop and update an effective drug in the short term to combat the constantly mutating virus. COVID-19 replicates RNA genome and transcriptional gene by RdRp complex. Unlike blocking spike proteins to combat viral infections, the use of high affinity aptamers that compete with natural substrates of RdRp may render viral replication ineffective.

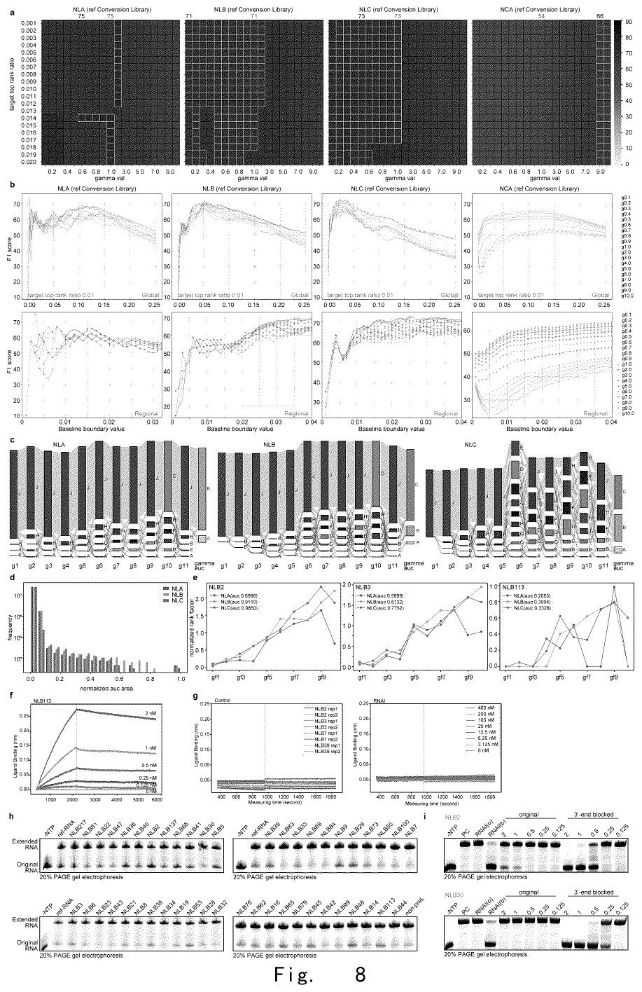

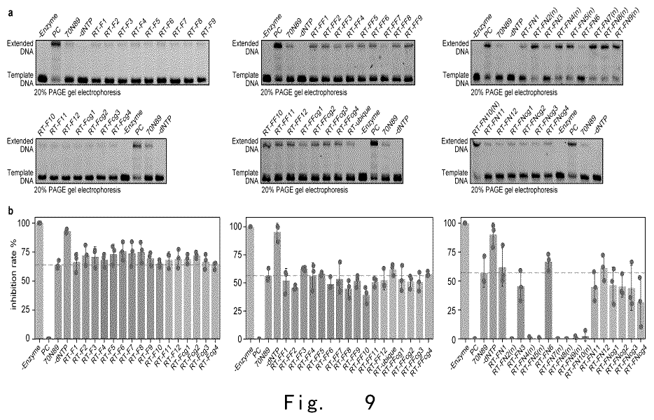

In order to explore whether this hypothesis is correct, the inventor first applied the method of the present invention and conventional methods to discover high-quality aptamers on the virus replicase nsp12, thereby generating three nsp 12 libraries of the present invention, namely magnetic bead blocking systems of type A (NLA), type B (NLB), and type C (NLC). At the same time, three conventional nsp12 libraries were established, namely type A (NCA), type B (NCB), and type C (NCC), with the same blocking system. Similar to the SiR library of the present invention, NLA (75), NLB (71), and NLC (73) exhibit higher f1 scores compared with the conventional library NCA (54) (FIG. 8a). Similarly, using the default gamma value (1.0) also performed well in predicting the top ranked aptamers (FIG. 8b). According to the enrichment route of the top ranked aptamers in the present invention, the difference in enrichment ability is small and follows the order of NLC>NLB≥NLA (FIG. 8c). In addition, although the AUC scores of NLB and NLC are similar (FIG. 8e), the NLB library still has denser predicted top-level aptamer sequences than other libraries (FIG. 8d). It is worth noting that a high proportion of top ranked aptamers bind to nsp12 and can be found on all three aptamers (FIGS. 7a and 7b). The top ranked aptamer NLB2 showed a K_{D} of 827 pM, while the lower ranked aptamer NLB113 even showed a K_{D} of 32 pM (FIG. 7c and FIG. 8f). On the contrary, both the RNA background control system without nsp12 and the background control system with random RNA and nsp12 did not show any significant signals (FIG. 8g). To further evaluate the binding ability of the top ranked aptamer, 86 out of the top 200 were randomly selected from the NLB library. More than 90% of them were found to have strong binding affinity with nsp12, and over half of these binding aptamers had KD below 10 nM (FIG. 7d). These high affinity aptamers rank higher in the present invention than in conventional libraries (FIG. 7e). This indicates that the method of the present invention can more effectively enrich high-quality aptamers than traditional methods.

Meanwhile, the inventor studied the inhibitory effects of these high affinity aptamers by competing with RdRp extension reaction for RNA substrates. NLB2 showed a higher inhibitory effect than other aptamers (FIG. 8h). It is valuable that when the 3′-end of RNA aptamers were blocked by oxidation can significantly enhance their inhibitory effect (FIG. 8i). Compared with the NLB2 aptamer, NLB30 has a lower inhibitory effect before blocking the 3′-end, but a higher inhibitory effect after blocking. Such change in the blocking effect suggests that a RNA aptamer may bind to the entire complex of nsp12 and/or RdRp in different regions, however, the main inhibitory function depends on the RNA 3′-end. In addition, when the number of RNA aptamer molecules is less than that of nsp12 protein, the complete inhibitory effect will be weakened. This indicates that the RNA inducer with the 3′-end being blocked interacts with nsp12 in a 1:1 ratio. In addition, multiple RNA aptamers (such as NLB30) can inhibit over 98% of viral replication extension reactions, while the control RNA does not (FIGS. 7f and 7g). Moreover, this strong inhibitory effect remained consistent over a period of time (FIGS. 7h and 7i). Therefore, based on the inhibition of RNA-nsp12, the method of the invention can quickly screen high affinity aptamers to inhibit the replication of rapidly mutated COVID-19.

In addition, considering that chemical modifications such as fluorination can improve the stability of RNA, unconventional RNA aptamer screening is also validated in the present invention. Based on HIV-1 replicase as a screening target, three different screening libraries are established in the present invention, including a single length library (“RT-F”) and mixed length libraries (“RT-FF”, “RT-FN”). Top 12 RNA aptamers screened from these libraries can efficiently inhibit the replication efficiency of HIV-1 reverse transcriptase (FIG. 9), and other aptamers containing the reported “CGGG” paired inhibition domain also have inhibitory effects. Therefore, this method has a reliable screening effect on RNA chemical modification. In a specific embodiment, the RNA aptamer of the present invention may include chemically modified sequences. In a preferred embodiment, the chemically modified sequence is a fluorine-modified sequence.

In a specific embodiment, the present invention provides a method for screening RNA aptamers, comprising following steps:
1) providing a library of RNA aptamers to be screened;
2) incubating the library of step 1) with a solid carrier fixed with a target, thereby inducing the RNA aptamer in the library to sufficiently bind to the target;
3) adopting a buffer gradient to elute the RNA aptamers bound to the target on the solid carrier in step 2), and collecting the eluate for each elution, respectively;
4) completely eluting the RNA aptamers still retained on the solid carrier after step 3), and collecting the eluate as the last group of eluate;
5) optionally concentrating and purifying the RNA aptamers in the eluates obtained in steps 3) and 4);
6) reverse-transcribing the RNA aptamers obtained in step 5) to obtain cDNAs;
7) amplifying and high-throughput sequencing the cDNAs obtained in step 6) to obtain sequencing data;
8) analysing the sequencing data obtained in step 7) and sorting RNA aptamer candidate sequences according to their binding potential from high to low, thereby obtaining high-affinity RNA aptamer sequences.

Based on the teachings of the present invention, a skilled person will be aware that RNA aptamer libraries used for screening can be libraries from various sources, including but not limited to the RNA aptamer library prepared in-house, commercially available RNA aptamer library, or RNA aptamer library as a gift from another person.

The solid carrier used in the method of the present invention can be any solid carriers known to a skilled person, including but not limited to: magnetic beads, matrix, and the like. In a preferred embodiment, the matrix includes, but is not limited to: agarose gel matrix, cephalosporin beads, nitrocellulose, polyvinylidene difluoride membranes, octyl alginate, and other carrier matrices.

The method of the present invention is suitable for screening RNA aptamers that bind to various targets. In a specific embodiment, the target can be a small molecule, including but not limited to: steroids, dopamine, kanamycin, digoxin, antoxin, dinitroaniline, melamine, quinolone, aflatoxin; or can be a large molecule, including but not limited to: polypeptides, proteins (e.g., enzymes and antibodies, etc.) and complexes (proteins bound with RNA), macromolecules and compounds, and the like.

When using a buffer gradient to elute the RNA aptamers bound to the target on the solid carrier, a buffer of increased volume, or a buffer of increased elution strength can be used; and preferably a buffer of increased volume is used for the gradient elution.

In a preferred embodiment, the buffer with increased elution strength is a buffer that prevents the RNA from folding to form a spatial structure by for example, increasing the concentration of salt ions or chelating agents.

For facilitating the subsequent high throughput sequencing, prior to the gradient elution, several background elutions are performed until the number of molecules of RNA aptamer contained in the eluate is not greater than 1% of the high throughput sequencing threshold. The volume of buffer for background elution should be not greater than the initial volume of buffer used for gradient elution.

In a specific embodiment, the elution may be a static elution (discontinuous elution, collecting the complete eluate at once) or a dynamic elution (continuous elution, continuously collecting a small amount of partial eluate), preferably a static elution. A skilled person will know the technical means for achieving the static and dynamic elution. For example, the static elution can be achieved by soaking magnetic beads in a buffer solution; alternatively, the dynamic elution can be achieved by flowing a buffer through a solid carrier, similar to column chromatography. In a preferred embodiment, when a static elution is used, the last background elution is performed in a new vessel. The volume of the buffer for background elution may or may not be increased, preferably not increased. The buffer for background elution and the buffer for gradient elution may be the same or different; preferably the same.

In a specific embodiment, the buffer for the gradient elution comprises magnesium ions, preferably 5 mM magnesium ions, a pH below 8.5, preferably pH 7-8, and a concentration of NaCl or KCl between 75 mM-200 mM.

The number of background elution can be determined based on the actual size of the library and subsequent sequencing throughput. For example, if the number of molecules in the RNA aptamer library to be screened is 10¹⁴, and the flux of the selected NextSeq sequencing is approximately 10⁷ sequences/group of eluate, the number of molecules needs to be reduced to less than 1% of the sequencing flux (if 10⁷ sequences are sequenced for 10⁷ molecules, an average of 1 sequence/molecule will be obtained, while if 10⁷ sequences are sequenced for <10⁵ molecules, an average of 100 sequences/molecule will be obtained, thereby reducing the probability of obtaining a reading of 1 sequence/molecule due to random sampling), that is, 10⁵⁼¹⁴⁻⁷⁻² or less. If the elution volume is 200 μL for each time and the residual elution volume (including solid phase carrier) is around 2 μL, the elution intensity is 200/2=10². Therefore, at least 5 background elutions of 10² are required to reduce the library from 10¹⁴ to below 10⁵.

Similarly, multiple gradient elutions are performed to ensure that the number of RNA aptamer molecules in the eluate is suitable for sequencing, preferably to lower the theoretical minimum number of molecules in the library to below 10⁵ and complete elution will be performed to completely remove the RNA aptamers bound to the target on the solid carrier.

Based on the molecular weight and length, a skilled person can understand the number of molecules in the library to be screened, and thus determine the number of background elutions and gradient elutions in advance. In a specific embodiment, 1-2 additional background elutions and gradient elutions can be added to the predetermined number of elutions.

In the subsequent gradient elution, if the static elution is used, as the elution volume increases, the system screening pressure also increases, and the elution liquid level is always higher than the previous elution liquid level, thereby reducing the contamination on the container wall.

Compared with the existing technologies, the advantage of the present invention is that it will not lose any RNA aptamer information in the library. Therefore, the method of the present invention not only retains the eluate solution obtained from the background elution and gradient elution mentioned above, but also performs complete elution after gradient elution, thereby completely eluting the RNA aptamer bound to the target on the solid carrier. Accordingly, the buffer for the complete elution contains reagents capable of releasing the RNA aptamer, including reagents capable of disrupting the binding of the target to the solid carrier, and/or reagents capable of disrupting the binding of the RNA aptamer to the target, and/or reagents directly disrupting the target. A skilled person can independently decide or select reagents that can release RNA aptamers based on specific situations. For example, if a small molecule target is linked to a solid carrier coated with streptavidin via biotin, the buffer used for complete elution contains reagents that separate the small molecule target from the solid carrier, such as DTT, and EDTA that separates the RNA aptamer from the small molecule target. For another example, if the target is a large molecule, such as a protein, reagents that can disrupt the protein, such as proteases, can be directly used to separate RNA aptamers from the target. When using a protease, it is important to note that the presence of the protease should not have adverse effects on subsequent reverse transcriptase activities. For example, protease inhibitors can be further used or temperature sensitive proteases can be utilized to inactivate the protease by heating after complete elution, without affecting the reverse transcriptase activity in subsequent steps.

After obtaining the eluate solutions obtained from background elution, gradient elution, and complete elution, the RNA aptamers in the obtained eluate solutions can be concentrated and purified. Whether the RNA aptamers in the eluate shall be concentrated and purified can be determined by a skilled person based on specific requirements. Without concentration or purification, the instrument can directly perform reverse transcription reaction on the RNA aptamers in the eluate, however the volume of the solution is large and the reagents will be wasted. If the RNA aptamers in the eluate are concentrated and purified, it can save reagents, but the concentration time can take several hours.

As mentioned above, when amplifying and sequencing RNA aptamers in the eluate, the inventor designed offset PCR by randomly inserting 0-6 nt compensating sequences between the sequencing linker and the constant region of cDNA due to the base imbalance, However, after using the 0-6 nt compensating sequence, the amplified DNA still has a small number of bases in an unbalanced distribution (for example, A is missing at the 10^{th} base of the 5′-end fixed sequence, and A is also missing at the 11^{th} to 13^{th} bases, as shown in the VI sequencing 5′-end start sequence (marked with dark green and orange letters) in FIG. 2d, G is missing at the 13^{th} to 14^{th} positions of the 3′-end fixed sequence, and T is missing at the 8^{th} to 12^{th} positions of the intermediate pre loop region). Therefore, it is necessary to add random sequences having approximately the average length of the library to the sequencing library, and use fixed sequences that are missing from the library at imbalanced positions (such as at the 5′-end, using random N at 1^{st}_7^{th} bases, using G at 8^{th} base to avoid consecutive 6 identical fluorescent sequencing signals, using fixed base A at 9^{th}-13^{th} bases; at the 3′-end, using random N at 1^{st}_7^{th} bases, and using C complementary to C at 13^{th} base). In brief, if there are still missing bases in the sequence after adding offset, a customized sequence can be used to supplement the missing bases. For positions where no bases are missing, a random N can be used in the customized sequence.

Therefore, in the amplification and sequencing steps of the method of the present invention, a custom designed PhiX is also introduced during the mixing of multiple samples to further compensate for the unbalanced base distribution in the constant region.

In addition to not losing any RNA aptamer information in the library, the method of the present invention considers the binding potential of RNA aptamer candidate sequences for sorting during sequencing data analysis, thereby obtaining RNA aptamer sequences with high binding affinity. In a specific embodiment, the binding potential refers to the rapid increase in enrichment level in each eluate, rather than solely considering the highest enrichment level. In a preferred embodiment, the binding potential is determined based on one or more of the following information about RNA aptamers: the abundance of RNA aptamers present in each eluate, the frequency at which a RNA aptamer is detected individually in each eluate, and a RNA aptamer is better when appears in a subsequent eluate than in the initial eluate. In a preferred embodiment, a standard curve is fitted based on the above information to evaluate the binding potential of RNA aptamers according to the area under the curve (AUC).

Based on the RNA aptamer screening method of the present invention, the present invention provides an apparatus for implementing the method of the present invention. Based on the teachings of the present invention, a skilled person can understand how to construct an apparatus for implementing the method of the present invention. For example, the apparatus may include modules for performing the steps of the method of the present invention.

Based on the teachings of the present invention, a skilled person will know that the RNA aptamers screened and obtained by using the method of the present invention can be prepared into various products. For example, in a specific embodiment, the RNA aptamers screened and obtained by using the method of the present invention can be prepared into biochips. In other embodiments, the RNA aptamers screened and obtained by using the method of the present invention can be prepard into a pharmaceutical composition.

A skilled person will know that if the RNA aptamers screened and obtained by using the method of the present invention have high binding affinity for specific recognition receptors on the cell surface, the RNA aptamers of the present invention can be attached to liposomes, thereby enabling specific delivery of drugs within liposomes to designated cells. Therefore, the RNA aptamers screened and obtained by the method of the present invention can be prepared into drug delivery carriers. In a specific embodiment, the drug delivery carrier is a liposome.

In other embodiments, the RNA aptamers screened and obtained by using the method of the present invention can also be prepared into diagnostic reagents.

### Beneficial Effects of the Present Invention

1. The false positive rate of the library is low (as low as 0%);
2. The aptamers screened from the library have strong binding ability (K_{D} 25 nM siR small molecule, 32 pM nsp 12 large molecule);
3. The time for preparing a library is short, one round of screening (about 4 hours);
4. The reproducibility of library preparation is high; and
5. Library preparation can be further optimized for the application of automated robotic arms (96 samples).

The present invention will be further explained in conjunction with specific embodiments. It should be understood that these embodiments are only used to illustrate the present invention and not to limit the scope of the present invention. The experimental methods without specific conditions specified in the following examples are usually carried out under conventional conditions or conditions recommended by the manufacturer. Unless otherwise specified, percentages and portions are calculated by weight.

### EXAMPLES

### Example 1: Preparation of RNA Screening Library Source

### 1.1. Random RNA Screening Library Source

### 1.1.1 Synthesis of Single Stranded DNA Template

A 103 nt ssDNA template library was customized from a primer reagent company, wherein the template (5′-3′ end) consists of primer A binding region (19 nt) [Famulok, M. Molecular Recognition of Amino Acids by RNA Aptamers: An L-Ccitrulline Binding RNA Motif and Its Evolution into an L-Arginine Binding. J Am Chem Soc 116, 1698-1706, doi: 10.1021/ja00084a010(2002)], left arm random region (26 nt), Pre-formed Loop Fixation Region (12 nt) [Davis, J. H.&Szostak, J. W. Isolation of high-intensity GTP aptamers from partially structured RNA libraries. Procedure Natl Acad Sci USA 99, 11616-11621, doi: 10.1073/pnas. 182095699 (2002)], right arm random region (26 nt), primer B binding region (20 nt) [Famulok, M. Molecular Recognition of Amino Acids by RNA Aptamers: An L-Ccitrulline Binding RNA Motif and Its Evolution into an L-Arginine Binding. J Am Chem Soc 116, 1698-1706, doi: 10.1021/ja00084a010(2002)] (see FIG. 2a). During the synthesis process, the random region uses artificially pre-mixed nucleotide substrates with an A:C:G:T ratio of 3:3:2:2. The entire reaction was commercially synthesized at a scale of 1 μmol.

### 1.1.2. Purification of Single Stranded DNA Template

10% polyacrylamide PAGE gel (20 cm*40 cm) was used to purify the ssDNA template library (˜200 μg, 0.5 mL volume), 20 W, 1.5 h. Under a UV (254 nm for DNA imaging, 365 nm for RNA imaging) lamp, the by-products in the electrophoretic separation and synthesis process wrere distinguished, the main product was labelled, and the gel was cut and dissolved in 5 mL of 0.3M NaOAc (pH 5.5) with rotating (550 rpm) overnight. 3 times the volume of EtOH was added for precipitation overnight, centrifuged at 4° C. (14000 g) for 30 minutes. The precipitate was rinsed with 2 mL of 70% EtOH, and centrifuged at 4° C. (14000 g) for 10 minutes. The rinsing operation was repeated with 0.5 mL of 70% EtOH, and finally the DNA precipitate was dried at room temperature. The precipitate was dissolved in TE buffer (10 mM Tris HCl, 1 mM EDTA, pH 8.0).

### 1.1.3. Amplification of Double Stranded DNA Template

0.16 μM purified ssDNA, 5 μM primer A (see Table 1), 5 M primer B (see Table 1), 1×Taq buffer (10 mM Tris HCl, PH 8.4, 50 mM KCl, 0.1% (v/v) Triton X-100, 500 μM dNTPs, 6 mM MgCl₂, and 0.1 U/μL Taq DNA polymerase, with a total reaction volume of 30 mL. The PCR reaction is first heated to 94° C. (3 minutes), followed by 6 cycles (each cycle includes denaturation at 94° C. for 1 minute, annealing at 52°° C. for 1 minute, and extension at 72° C. for 3 minutes), and finally the extension step (72° C., 20 minutes), and cooled to 4° C.

### 1.1.4. Purification of Double Stranded DNA Template

An equal volume of P/C/I reagent was added and vortexed for 30 seconds, and centrifuged at 14000 g at room temperature for 20 minutes. The aqueous supernatant was transferred to a new 50 mL centrifuge tube. Then an equal volume of chloroform was added, vortexed, and centrifuged. The aqueous phase was transferred. And the chloroform extraction process was repeated. Finally, dsDNA was precipitated in 70% EtOH, 0.3 M NaOAc, and the precipitate waswashed with ethanol, dried, and dissolved in 200 μL TE buffer.

### 1.1.5. In Vitro Transcription of RNA

534 nM purified dsDNA was incubated in 1× transcription buffer (40 mM Tris HCl pH 8.1, 1 mM spermidine, 22 mM MgCl₂, 0 01% Triton X-100, along with 10 mM DTT, 5% (v/v) DMSO, 1 U/mL pyrophosphatase (optional addition), 4 mM ATP/CTP/GTP/UTP, 40 μg/mL acetylated BSA (Sigma Aldrich) (optional addition), 0.7 μM T7 polymerase (Thermo Fisher Scientific), with a total reaction volume of 10 mL. For the in vitro transcription reaction of 2′-hydroxyfluorinated RNA, the same reaction system was used except that CTP and UTP substrates were replaced with the same concentration of 2′-F-CTP (Jena Bioscience) and 2′-F-UTP (Jena Bioscience), and T7 polymerase was replaced with the same concentration of mutated T7 polymerase (Y639F, in-house prepared), while keeping other components unchanged. In vitro transcription reaction was incubated at 37° C. for 4 hours. Subsequently, 50 U/mL DNase I was added and the reaction was incubated at 37° C. for another hour. P/C/I purification and two times of chloroform extractions were performed, followed by isopropanol precipitation at −20° C. for 1 hour and two times of washes with 75% ethanol, so that the desired RNA was obtained. Then the RNA was dissolved in dH2O and stored at −20° C. or −80° C. for a long time.

### 1.2. Preparation of Directed Random RNA Library Source

The process of preparing a directed random RNA library source is similar to the preparation of a random RNA library source, except for the synthesis of ssDNA templates and PCR amplification and purification of dsDNA.

### 1.2.1. Synthesis of Single Stranded DNA Template

When synthesizing the ssDNA template library, based on known ancestral sequence information (103 nt, see Table 1), the corresponding random regions were commercially synthesized at a scale of 1 μmol using artificially pre-mixed nucleotide substrates, with a ratio of 85:5:5:5 between the original base and the other three non original bases.

### 1.2.2. Amplification of Double Stranded DNA Template

For the amplification of dsDNA, 50 nM ssDNA was used as a template and PCR amplification was performed in 1×Taq buffer, along with 5 μM primer A, 5 μM primer B, 500 μM dNTPs, 1.5 mM MgCl₂, and 0.05 U/μL Taq DNA polymerase in a total volume of 1 mL. The PCR process is the same as the preparation of a random RNA library source.

### 1.2.3. Amplification of Double Stranded DNA Template

PCR products were purified by using QIAquick PCR Purification Kit (QIAGEN). 400 nM of purified dsDNA was subjected to in vitro transcription reaction in a total volume of 2 mL. After being purified by PAGE, RNAs were dissolved in dH₂O and stored under the same storage conditions as described above.

### Example 2. Screening and Sequencing of RNA Aptamers

### 2.1. RNA Advanced Structure Folding

RNA from random or directed random library sources, yeast-tRNA (Invitrogen), and competitive RNA (see Table 1) were incubated at 75° C. for 5 minutes, then slowly cooled to 4° C. at a rate of 0.1° C./s, and placed on ice.

2.2.a. RNA Screening RNA Binding to Small Molecules (Using Biotin Labeled Silicon Rhodamine as an Example)
2.2.1.a. Pre-Balanced Magnetic Beads

hydrophilic streptomycin magnetic beads (New England Biolabs) were enriched using a 6-tube magnetic separation rack (New England Biolabs), washed and equilibrated for 5 times with 4 times the volume of magnetic beads of 1×ASB buffer (20 mM HEPES pH 7.4125 mM KCl, 5 mM MgCl₂), and then resuspended in 0.5 volume of magnetic beads of 1×ASB buffer.

2.2.2.a. Blocking magnetic bead background: 5 μM of biotinylated silicon rhodamine and 25% (v/v) concentrated magnetic beads (2.1. a) were incubated in 100 μL of 1×ASB buffer: Group A, with no additional reagents being added (referred to as “+Ne”, RLA); or 0.15 μg/μL of folded yeast tRNA being added as Group B (referred to as “+tRNA”, RLB); or 4 μM folded competitor RNA 1 (see Table 1) being added as Group C (referred to as “+cRNA”, RLC). After being thoroughly mixed, the reaction was incubated at 25° C. for 30 minutes at a speed of 1000 rpm.

2.2.3.a. Balancing blocked Magnetic Beads

The magnetic beads were washed for five times with 200 μL of 1×ASB buffer solution, gently mixed the magnetic beads during each wash, stood at room temperature for 30 seconds, and then placed in a magnetic rack. The liquid was removed, and 1×ASB buffer solution was quickly added to avoid drying the magnetic beads.

2.2.4.a. Binding RNA

4 μM of RNAs from a random library source was prepared in advance and dissolved in 100 μL of 1×ASB buffer. After being washed and blocked, the magnetic beads were resuspended in the RNA solution, mixed thoroughly, and incubated at 25° C. and 1000 rpm for 1 hour.

2.2.5.a. Washing RNA by gradient Screening

Magnetic beads were collected by using a magnetic rack, the supernatant was removed, and then the RNA-bound magnetic beads were washed with 200 μL of 1×ASB buffer for 4 times. Each eluate was collected separately, and the magnetic beads were let stood for 30 seconds, placed in the magnetic rack, then resuspended in 200 μL of 1×ASB buffer, and transferred to a new 1.5 mL centrifuge tube. The magnetic beads were enriched with the magnetic rack, the 5^{th} eluate was collected, and washed with 250 μL, 300 μL, 350 μL, 400 μL and 450 μL of 1×ASB buffer for 5 times in sequence. Similarly, each eluate was collected separately, 10 times in total. Finally, complete elution is performed: first, the magnetic beads were incubated with 200 μL of 50 mM DTT solution at 25° C. for 20 minutes at a speed of 650 rpm. The eluate was collected and combined with the subsequent second eluate as the 11^{th} elution. The second eluate was incubated with 100 μL of 50 mM DTT and 5 mM EDTA at 25° C. for 5 minutes at a speed of 650 rpm.

2.2.6.a. Concentration and Purification of RNA

3 times the volume of cooled EtOH, 0.1 times the volume of 3 M NaOAc, and 1 μL glycogen (Thermo Fisher Scientific) were added, and 10 eluates and 1 combined eluate from 2.5.a were precipitated at −20° C. for 2 hours or overnight. The precipitated RNA was centrifuged (4° C., >20000 g, 1 h), washed, and dissolved in 10 μL dH₂O. [Optional step: 2.4.a The reaction volume of the RNA binding step can be correspondingly reduced to 20 microliters. In the second elution step of 2.5.a, 5 mM EDTA can be omitted. At this time, these 10 eluates and 1 combined eluate can be directly added to the reverse transcription reaction without the need for RNA precipitation in 2.6. a]

2.2.b. Screening RNA Binding to Macromolecules (Taking His Labeled COVID-19 Replicatase as an Example)
2.2.1.b. Pre-Balancing Magnetic Beads

A 6-tube magnetic separator (New England Biolabs) was used to enrich HisPur™ Ni-NTA magnetic beads (Thermo Fisher Scientific), washed and equilibrated for 5 times with 4 times the volume of magnetic beads of 1×ERB buffer (100 mM NaCl, 20 mM Na HEPES pH 7.5, 5% (v/v) glycerol, 10 mM MgCl₂, and 0.5 mM B-mercaptoethanol (optional addition)), and then resuspended in 0.3 times the volume of magnetic beads of 1×ERB buffer.

2.2.2.b. Blocking Magnetic Bead Background

50% (v/v) concentrated equilibrium magnetic beads (2.1. a) was added to 30 μL of 1×ERB buffer: Group A, with no additional reagents being added (referred to as “+Ne”, NLA); or 0.4 μg/μL of folded yeast tRNA being added as Group B (referred to as “+tRNA”, NLB); or, 10 μM folded competitive RNA 2 (see Table 1) being added as Group C (referred to as “+cRNA”, NLC). After being thoroughly mixed, the reaction stood at 25° C. for 2 minutes, and then 50 μL of 30 μM nsp12 dissolved in 1×ERB buffer was added, mixed gently, incubated at 25° C. for 10 minutes, and gently mixed with tapping by fingers.

2.2.3.b. Balancing Blocked Magnetic Beads

The magnetic beads were washed for five times with 200 μL of 1×ERB buffer, gently mixed the magnetic beads for each wash, stood at room temperature for 30 seconds, and then placed in a magnetic rack. The liquid was removed, and 1×ERB buffer was quickly added to avoid drying the magnetic beads.

2.2.4.b. Binding RNA

4 μM of RNAs from a random library source was prepared in advance and dissolved in 100 μL of 1×ASB buffer. After being washed and blocked, the magnetic beads were resuspended in the RNA solution, mixed thoroughly, incubated at the room temperature for 1 hour with tapping by fingers, then incubated at 37° C. for 5 minutes, and finally, incubated at 25° C. for 2 minutes.

2.2.5.b. Washing RNA by Gradient Screening

Magnetic beads were collected by using a magnetic rack, the supernatant was removed, and then the RNA-bound magnetic beads were washed with 200 μL of 1×ASB buffer for 4 times. Each eluate was collected separately, and the magnetic beads were let stood for 30 seconds, placed in the magnetic rack, then resuspended in 200 μL of 1×ASB buffer, and transferred to a new 1.5 mL centrifuge tube. The magnetic beads were enriched with the magnetic rack, the 5th eluate was collected, and washed with 250 μL, 300 μL, 350 μL, 400 μL and 450 μL of 1×ASB buffer for 5 times in sequence. Similarly, each eluate was collected separately, 10 times in total. Finally, complete elution is performed: Firstly, the beads were incubated in 400 μL 1×ERB buffer containing 0.1 U/μL proteinase K (New England Biolabs) and 2 mM CaCl₂ at 37° C. for 45 minutes with gently tapping by fingers. The eluate was collected and combined with the subsequent second eluate as the 11^{th} elution. The second eluate was incubated in 100 μL of 1×ERB buffer at room temperature for 1 minute, and then recovered on a magnetic rack. The volume of the combined eluate was supplemented to 500 μL and subjected to two times of P/C/I extraction and purification, so as to recover the supernatant.

### 2.2.6.b Concentration and Purification of RNA

3 times the volume of cooled EtOH, 0.1 times the volume of 3 M NaOAc, and 1 μL glycogen (Thermo Fisher Scientific) were added, and 10 eluates and 1 combined eluate from 2.5.a were precipitated at −20° C. for 2 hours or overnight. The precipitated RNA was centrifuged (4° C., >20000 g, 1 h), washed, and dissolved in 10 μL dH₂O. [Optional step: 2.4.b The reaction volume of the RNA binding step can be correspondingly reduced to 20 microliters. In the complete elution step of 2.5.6, a thermosensitive protease K (New England Biolabs) with a concentration of 0.015 U/μL can be used to replace protease K, and incubated at 25° C. for 1 hour with tapping by fingers. Then the reaction system was incubated at 55° C. for 10 minutes to inactivate thermosensitive protease K. At this point, these 10 eluates and 1 combined eluate can be directly added to the reverse transcription reaction without the need for 2.6.b RNA precipitation]

### 2.3. Reverse Transcription of RNA

For reverse transcription with a final volume of 20 μL, into RNA of 2.6 were added 0.5 M primer B, 0.5 mM dNTPs, and dH₂O and reacted at 65° C. for 5 minutes. After the reaction is completed, the system was immediately placed on ice and cooled for 2 minutes. Then, 1×SSIV buffer (Thermo Fisher Scientific), 5 mM DTT, and 10 U/μL SuperScript IV reverse transcriptase (Thermo Fisher Scientific) were added to the reaction, and the mixture was incubated at 53° C. for 1 hour.

2.4. Offset PCR dsDNA Compensation

In order to add compensating sequences to the library, 10 μL of the completed reverse transcription reaction was further PCR-amplified with 1×Taq buffer, 0.2 mM dNTPs, 3 μM offset selex frw primer_mix v1 (or v2), 3 μM offset selex frw primer mix v1 (or v2) (see FIG. 2d and Table 1), 2 mM MgCl₂, 0.05 U/μL Taq DNA polymerase, for a total volume of 50 μL. The PCR reaction is firstly heated to 94° C. (3 minutes), followed by 11 cycles (each cycle includes denaturation at 94° C. for 1 minute, annealing at 52.5° C. for 1 minute, and extension at 72° C. for 2 minutes), and finally the extension step (72° C., 5 minutes), cooled to 4° C.

2.5. Purification of Offset PCR dsDNA

Ampure XP magnetic beads (Beckman Coulter) were pre-equilibrated at room temperature for more than 30 minutes. In the PCR reaction, 1.2 times the reaction volume of magnetic beads was added, pipetted up and down for 40 times, thoroughly mixed and purified, stood at room temperature for 10 minutes, and placed on the SMARTer Seq PCR magnetic rack (Takara Bio). After being fully enriched, the magnetic beads were removed. The magnetic beads were washed in 180 μL 80% EtOH for two times, and dried at room temperature for about 5 minutes. 30 μL dH₂O was added, pipetted up and down and mixed thoroughly, stood at room temperature for 5 minutes, and placed on the magnetic rack. The dsDNA solution was collected, and the approximate concentration was measured by using a Nanodrop nucleic acid concentration analyzer (Thermo Fisher Scientific).

2.6. Labeling of Illumina PCR dsDNA

Sequencing primers and marker sequences were further added to dsDNA by PCR using ˜4.5 nM offset PCR dsDNA template, 500 nM sequencing universal primers (New England Biolabs), 500 nM sequencing index primers (New England Biolabs), 200 nM dNTPs, 1×Q5 reaction buffer (New England Biolabs), 0.02 U/μL hot start Q5 high fidelity DNA polymerase (New England Biolabs) and dH₂O, with a total volume of 50 μL. The PCR reaction is first heated to 98° C. (40 seconds), followed by 6 cycles (each cycle includes denaturation at 98° C. for 10 seconds, annealing at 68.5° C. for 20 seconds, and extension at 72° C. for 30 seconds), and finally the extension step (72° C., 2 minutes), and cooled to 4° C.

2.7. Purification of Illumina PCR dsDNA

Identical Step 5: dsDNA was dissolved in 15 μL of dH₂O and stored at −20° C.

### 2.8. Quality Control of Sequencing Library

1 μL of the sample was taken for precise concentration measurement using the Qubit Fluorometer (Invitrogen) method, and the sample concentration was determines as being greater than 2 ng/μL. Then, 1 μL of sample (diluted to 1-2 ng/μL) was taken for high-precision electrophoresis analysis quality control by Bioanalyzer (Agilent), and the single signal peak was determined as being around 225 bp.

### 2.9. Multi-Sample Sequencing

No more than 47 samples that have undergone quality control at the same concentration were mixed, into which 5% selexPhiX_v1 (corresponding to step 5 using offset_delex_primer v1) or selexPhiX_v2 (corresponding to step 5 using offset_delex primer v2) was added, diluted to a final concentration of 20 nM, diluted and denatured with NaOH. NextSeq 500 single ended (SE) high-throughput 75 bp sequencing was used with a sequencing density of 1.8 pM, and the 10 bp reagent used for sequence labeling was used for sequencing the sequence itself, resulting in a total sequence output of 86 bp and approximately 320-400 million sequences.

### Example 3. Data Modeling, Analysis, and Statistics

### 3.1. Data Cleaning

Based on the primer tag sequence (6 nt) used for each sample, the raw sequencing data were decoded and classified into corresponding sample sequences. During the process of corresponding label sequences, zero mismatch was taken as the standard, and low phred quality sequences were filtered off. Then, the compensating sequences (7 types, 0-6 nt) at the 5′-end of the sequence were cut off, and the balanced distribution of the compensating sequences was calculated. The corresponding sequence of primer B was further removed from the trimmed sequence, while retaining the 67 nt source sequence, and the source sequence was subjected to the reverse complementation to adjust it back to the DNA sequence consistent with the original RNA sequence. During the process of trimming sequences, background data without compensation sequences, without primer B sequences, with more than 25 consecutive identical bases, and with a trimmed length less than 65 nt were cleaned out.

### 3.2. Data Merging

A data box structure (12 columns*n rows) was created, wherein the first column is the sequencing sequence itself, columns 2-12 are the 10 groups of eluates and 1 group of combined eluate in 2.2.5 (groups 1-11, g1-11 in sequence), and each row represents an independently exclusive sequence statistic from a library. Based on the cleaned data from 3.1, the abundance of each sequence between groups 1-11 were calculated, and recorded in the data box structure. Subsequently, the parent sequences with more than 4 edit distances compared with the theoretical pre-formed ring sequence in the fixed area of the pre-formed ring region were removed from the merged data box. At the same time, the parent sequences with unknown “N” in the random areas of the left and right arms were removed. Finally, the merged data library of the standardized sequence was compressed and stored.

### 3.3. Data Conversion

In the merged database, for sequences with abundance of 0 in Group 1, the abundance was replaced with an initial non-zero value of 0.5. Then a fold change database (11 columns*m rows) was created, wherein the first column is the sequencing sequence itself, the second column is the Group 2 change ratio (f2), that is, the abundance of the sequence in Group 2 divided by its corresponding abundance in Group 1, the third column is the Group 3 change ratio (f3), that is the abundance of the sequence in Group 3 divided by its corresponding abundance in Group 1, and so on. For values with a ratio of 0, it will be replaced with 0.1. Finally, the logarithm of log2 was taken and the fold change database was saved.

3.4. Sorting modeling

In the fold change database, change ratios in each group were arranged from high to low and the sequence located at 1% (ruler percentile) was selected as the ruler passing sequence. Then, based on the initially set gamma trend line (constant c * interval ratio gamma-0.0000001), the default gamma value is 1, which generates 10 ratios of 0.1, 0.2, 0.3, . . . 0.9, and 1. The ratio of each passing sequence of the ruler was scaled to the corresponding ratio as a weighting process. For example, if the original ratio of the passing sequence of the first ruler is 5, all ratios of the first sequence will be divided by 50. If the original ratio of the passing sequence of the second ruler is 7,all ratios of the second sequence will be divided by 35, and so on. Finally, each sequence corresponds to 10 gamma change rates (gf) in 10 sets of change ratios, with 1-10 as the horizontal axis and the corresponding gamma change rates as the vertical axis, to calculate the area under the curve (AUC). The binding ability of the sequence can be predicted based on its AUC value. The larger the value, the stronger the potential binding ability, and vice versa.

3.5. Fine-tuning of Model

In the model, the hyperparameter gamma value and scale quantile can be adjusted based on the distribution and ratio of the original abundance of some potential strong binding sequences in each group, as well as the enrichment route, and can also be further optimized based on the Pearson correlation coefficient of the subsequences of the strong binding sequence. For small molecules, gamma ≥1 is recommended, and for large molecules, gamma ≤1 is recommended. It is recommended to use 1% for ruler division. For subsequence analysis, the first step is to select several strong binding sequences in the merged data box, split each sequence into a left arm random region, a pre-formed loop region, and a right arm random region, and further remove the primer A residue sequence from the left arm random region. A sliding window of size n (6-10) with a step size of 1 was applied in each region to calculate the abundance of subsequence features of each high abundance sequence, and the rich correlation coefficient trend was used as a reference for hyperparameters. In addition, when the binding strength of a small number of candidate sequences was measured, normalized loss cumulative gain (NDCG) can be used to further optimize the ranking related hyperparameters gamma values and scale quantiles.

### Example 4. Validation on Screen

### 4.1. Determination of Dissociation Constants for the Interaction Between RNA Aptamers and Silicon Rhodamine

The dissociation constant (KD) of RNA aptamers with silicon rhodamine was determined according to the JASCO fluorescence intensity at different RNA concentrations. In breif, RNA ligands underwent structural folding according to step 2.1, and then RNA was mixed with a 50 nM SiR-PEG2-NH2 probe in 1×ASB buffer in a fluorescence colorimetric dish at 25° C. The fluorescence intensity was recorded as the specified RNA concentration increases. The excitation and emission wavelengths were set to 647 nm and 662 nm, respectively, and the slit width for excitation and emission was set to ±5 nm. When calculating data, the Hill equation was used to simulate and combine curves to determine the dissociation constant.

### 4.2. Determination of RNA Aptamer-Activated Silicon Rhodamine Fluorescence and Live Cell Imaging

The RNA ligand (5 μM) was structurally folded according to step 2.1, and then the RNA solution was dissolved in 1×ASB buffer. 5 nM SiR-PEG2-NH2 probe was added and incubated at room temperature for 10 minutes. Fluorescence was measured using a JASCO spectrophotometer (λ ex=647 nm;) λ m=662 nm (±5 nm slit width)). For live cell imaging, Dulbecco's Modified Eagle's medium (DMEM, high glucose, phenol red free) (Gibco) was used to culture human embryonic kidney derived cells 293 (HEK293T), and an additional 10% fetal bovine serum (FBS) (Gibco), 100 U/mL penicillin (Thermo Fisher Scientific), and 100 μg/mL streptomycin (Thermo Fisher Scientific) were added to the culture medium. Partially activated cells were inoculated into 300 μL of culture medium and transferred to an 8-well glass chamber coated with poly-D-lysine for overnight growth. Then, FuGeneHD transfection reagent (Promega) was used to transfect cells with an appropriate amount of expression plasmid according to standard methods. After 48 hours, the culture medium was exchanged with Leibowitz (L15) medium containing 200 nM SiR-PEG-NH2. At 37° C., the cells were imaged, photographed, and subjected to corresponding visual adjustments.

### 4.3. Determination of the Dissociation Constant of the Interaction Between RNA Aptamer and COVID-19 Replicase

Based on the principle of biofilm interference, Octet® R8 system (Sartorius) was used to measure dissociation constants. Octet® Ni-NTA (NTA) biosensor (Sartorius) was equilibrated in 1×ERBL buffer (20 mM Tris HCl pH 7.4, 100 mM KCl, 5% (v/v) glycerol, 10 mM Mg (OAc) 2, 1 mM TCEP, 0.02% TWEEN 20 (Carl Roth)) for 5 minutes prior to dissociation constant measurement. The RNA in each well was diluted 2-fold sequentially from the specified concentration in 1×ERBL buffer, while the sample wells without RNA in 1×ERBL buffer were set as blank control group. Meanwhile, 20 ng/μL of His10-nsp12 was used for the protein loading step. The entire detection process includes a base-1 step of 60 second, a protein loading step of 180-240 second, a base-2 step of 60 second, a binding step of 900-1800 second, and a dissociation step of 600-3600 second. The measured data was analyzed by Octet data analysis software. In brief, the data is pre-processed according to reference subtraction, Y-axis alignment based on baseline mean, correction between dissociation steps, and Savitzky Golay filtering. A 1:1 combination model was used. Then the dissociation constant was calculated using the corresponding fitting method (local or global). The Ni NTA biosensor was reusable. In brief, the biosensor was washed repeatedly for three cycles of washing steps, including 10 mM glycine (pH 1.7) washing (10 seconds) and 1×ERB buffer neutralization (10 seconds), followed by 10 mM NiCl₂ regeneration (70 seconds) and 1×ERB buffer washing analysis (60 seconds), all of which were performed under shaking at 1000 rpm.

### 4.4. 3′-End Blocking Modification of RNA Aptamer

3.77 μM RNA aptamer was added to 200 μL of reaction solution, which contains 10 mM NaOAc pH 4.5, 50 mM freshly prepared NaIO₄, and dH₂O, and incubated at room temperature for 2 minutes. Then, 10% (v/v) ethylene glycol was added and mixed repeatedly, and let it stand at room temperature for 5 minutes to quench the oxidation reaction. Into the quenched reaction was further added 222 mM Tris HCl pH 8.9, 0.15 M NaOAc pH 5.5, 2 μL glycogen (Thermo Fisher Scientific), and 50% (v/v) isopropanol. The reaction mixture was incubated at room temperature for an additional 30 minutes. Finally, RNAs were precipitated by centrifugation (16000 g, 20 minutes, 4° C.) and washed twice with 75% EtOH. The RNAs were dissolved in dH₂O and stored at −20° C. or −80° C. for long-term preservation.

The sequencing library related sequences, RNA aptamer sequences, and control group sequences of the present invention are shown in Tables 1 and 2, respectively.

Wherein for siR small molecules, RLB7 (KD 250 nM), RLB15 (KD 194 nM), RLB3 (KD 208 nM), RLB4 (KD 195 nM), RLB8 (KD 700 nM), RLB12 (KD 370 nM), RLB13 (KD 461 nM), seqB (also RLB108, KD 25 nM) all exhibit excellent affinity.

For nsp12 protein, NLB113, NLB41, NLB30, NLB79, NLB34, NLB69, NLB32, NLB2,NLB58, NLB5 exhibit excellent affinity.

### Discussion

Since 1990, techniques for discovering high affinity aptamers have been developed in this field and good results have been achieved. However, the entire process of evolutionary selection is considered to operate a “black box” and typically takes several weeks or even months. Although high-throughput sequencing tools visualize the sequence selection in each round, they do not address the screening issue of high false positive rates for the selected aptamers. With the development of precision instruments and algorithms, the number of iterations in the screening process has decreased, and higher affinity and specificity aptamers have been generated. One promising “partitioning” method uses capillary electrophoresis for rapid screening of DNA aptamers. However, in addition to the requirments on complex instruments and manufacturing techniques, one limitation is that when an aptamer binds to a target with a molecular weight smaller than its own, it cannot generate sufficient mobility transfer signals to distinguish binding characteristics. Similarly, optimizing selection conditions of microfluidic chip separation systems (such as bead aggregation, microbubbles, RNA stability) for different binding targets is not an easy task. In addition, for predicting aptamer binding through computation, it mainly utilizes the subsequence and substructure information of RNA sequences. However, this type of data-driven analysis highly relies on manually selecting data corresponding to screening rounds and the quality of traditional screening experiments.

The RNA aptamer screening method developed in the present invention for small and large molecules only takes a few hours, that is, direct single round RNA screening without the need for stringent instruments, efficient deep sequencing library construction. The method of the present invention can be used for end-to-end analysis and is extremely easy to be used. In the method of the present invention, the characteristics of SGRELI maximize the useful information generated during the selection process. High affinity RNA aptamers can be observed for multiple times and exhibit a gradient sorting trend, therefore, the false positive rate of the predicted binding aptamer is low.

According to the above Examples, it can be seen that the SiR RNA aptamer screened by the method of the present invention has better KD and fluorescence activation ability. Compared with the best reported aptamer SIRA, the specificity is increased and the background of RNA live cell images is reduced. In addition, sequence length and composition can be further optimized based on structural interaction information. The nsp12 RNA aptamer with pM KD obtained by the method of the present invention provides a promising application prospect for inhibiting SARS-COV-2 polymerase replication. Further 3′-end blocking modification of the aptamer is a necessary condition for completely inhibiting polymerase elongation. Compared with remdesivir, the aptamer obtained by the invention achieves the same inhibitory effect at the same concentration of RdRp polymerase, and only requires one thousandth of the working concentration of remdesivir. The entry and occupation of the catalytic center of the replicase complex by RNA aptamers with a 3′-end structure may be necessary for inhibiting viral replication. Meanwhile, the present invention is applied to screen chemically modified RNA aptamers, and the obtained aptamers efficiently inhibit HIV-1 reverse transcriptase, further expanding the application of the present invention in screening. The method of the present invention can be further optimized to use machine learning and feature engineering (such as substructures, subsequences) to predict binding affinity, and can also use automated robots for high-throughput screening.

Summing up, the present invention emphasizes a method with SGRELI characteristics for rapid screening RNA aptamers, and provides a theoretical basis for the development of functional RNA aptamers that activate chemical dyes and inhibit SARS-COV-2 polymerase and HIV-1 reverse transcriptase.

All references mentioned in the present invention are cited as references in this application, as if each reference were cited separately. In addition, it should be understood that after reading the above teachings of the present invention, a skilled person can make various changes or modifications to the present invention, and these equivalent forms also fall within the scope of the claims attached to the present application.

TABLE 1
Sequencing library related sequences

| Name | Sequence (5′end→3′end | Use |
|---|---|---|
| Random Pool | GGAGCTCAGCCTTCACTGC-N₂₆- | Random library input RNA |
| | -CTGCTTCGGCAG-N₂₆-GGCACCACGGTCGGATCCAC (“N” is | template |
| | A:C:T:G = 25:25:25:25; That is, compared | |
| | with the original sequence, the nucleotide | |
| | represented by “N” belongs to a completely | |
| | random mutation) (SEQ ID NO: 1) | |
| Random 20F Pool | GGAGCTCAGCCTTCACTGC- | Random library input 2′-F |
| | -N₂₀-GGCACCACGGTCGGATCCAC (“N” is | modifed RNA template |
| | A:C:T:G = 25:25:25:25, 2′-F-CTP and | |
| | 2′-F-UTP was applied to the RNA pool | |
| | preparation) (SEQ ID NO: 2) | |
| Random 30F Pool | GGAGCTCAGCCTTCACTGC- | Random library input 2′-F |
| | -N₃₀-GGCACCACGGTCGGATCCAC (“N” is | modifed RNA template |
| | A:C:T:G = 25:25:25:25, 2′-F-CTP and | |
| | 2′-F-UTP was applied to the RNA pool | |
| | preparation) (SEQ ID NO: 3) | |
| Dope seqA Pool | GGAGCTCAGCCTTCACTGC-CGCCCCCACCGGGTTTGAAAAC | Validation library input |
| | CTGG-CTGCTTCGGCAG-TTGTATCCTTTGGGGCTCGGCAATT | RNA template |
| | C-GGCACCACGGTCGGATCCAC (“N”: other three | |
| | non-Ns = 85:5:5:5; That is, compared with | |
| | the original sequence, the nucleotide | |
| | represented by “N” belongs to directed | |
| | mutation, with 85% identical to the | |
| | original sequence) (SEQ ID NO: 4) | |
| Dope seqB Pool | GGAGCTCAGCCTTCACTGC-AAGATGTGGACCATTTAACTTGT | Validation library input |
| | AGA-CTGCTTCGGCAG-GCGGCTGTTCCCTCAAGGGAACGCTT- | RNA template |
| | GGCACCACGGTCGGATCCAC (“N”: other three | |
| | non-Ns = 85:5:5:5) (SEQ ID NO: 5) | |
| Dope seqC Pool | GGAGCTCAGCCTTCACTGC-CAGACCGCGTTTAGAAACGCGT | Validation library input |
| | AAAT-CTGCTTCGGCAG-ATTGATTACTATCGATCTGGTAACGA- | RNA template |
| | GGCACCACGGTCGGATCCAC (“N”: other three | |
| | non-Ns = 85:5:5:5) (SEQ ID NO: 6) | |
| SiR-CRNA | GCUGCGACGUUUGAAAACGUCUAACUGCUUCGGCAGAAC | Library group C blocker |
| | GGUAUCCCGGCGGC (SEQ ID NO: 7) | |
| nsp12-cRNA | UUUUCAUGCUACGCGUAGUUUUCUACGCG (SEQ ID NO: 8) | Library group C blocker |
| Primer A | TCTAATACGACTCACTATA GGAGCTCAGCCTTCACTGC | Library PCR |
| | (SEQ ID NO: 9) | |
| Primer B | GTGGATCCGACCGTGGTGCC (SEQ ID NO: 10) | Library PCR and RT |
| SS_F_v1 | ACACGACGCTCTTCCGATCT CGACCGTGGTGCC (SEQ ID | Offset PCR v1 |
| | NO: 11) | |
| ES_F1_v1 | ACACGACGCTCTTCCGATCT A CGACCGTGGTGCC (SEQ ID | Offset PCR v1 |
| | NO: 12) | |
| ES_F2_v1 | ACACGACGCTCTTCCGATCT TA CGACCGTGGTGCC (SEQ | Offset PCR v1 |
| | ID NO: 13) | |
| ES_F3_v1 | ACACGACGCTCTTCCGATCT GTA CGACCGTGGTGCC (SEQ | Offset PCR v1 |
| | ID NO: 14) | |
| ES_F4_v1 | ACACGACGCTCTTCCGATCT CCAT CGACCGTGGTGCC | Offset PCR v1 |
| | (SEQ ID NO: 15) | |
| ES_F5_v1 | ACACGACGCTCTTCCGATCT ACTTA CGACCGTGGTGCC | Offset PCR v1 |
| | (SEQ ID NO: 16) | |
| ES_F6_v1 | ACACGACGCTCTTCCGATCT GTACTT CGACCGTGGTGCC | Offset PCR v1 |
| | (SEQ ID NO: 17) | |
| SS_R_v1 | AGACGTGTGCTCTTCCGATCT GCTCAGCCTTCACTGC (SEQ | Offset PCR v1 |
| | ID NO: 18) | |
| ES_R1_v1 | AGACGTGTGCTCTTCCGATCT T GCTCAGCCTTCACTGC | Offset PCR v1 |
| | (SEQ ID NO: 19) | |
| ES_R2_v1 | AGACGTGTGCTCTTCCGATCT AT GCTCAGCCTTCACTGC | Offset PCR v1 |
| | (SEQ ID NO: 20) | |
| ES_R3_v1 | AGACGTGTGCTCTTCCGATCT CAT GCTCAGCCTTCACTGC | Offset PCR v1 |
| | (SEQ ID NO: 21) | |
| ES_R4_v1 | AGACGTGTGCTCTTCCGATCT GCAT | Offset PCR v1 |
| | GCTCAGCCTTCACTGC (SEQ ID NO: 22) | |
| ES_R5_v1 | AGACGTGTGCTCTTCCGATCT AGCAT | Offset PCR v1 |
| | GCTCAGCCTTCACTGC (SEQ ID NO: 23) | |
| ES_R6_v1 | AGACGTGTGCTCTTCCGATCT CATACT | Offset PCR v1 |
| | GCTCAGCCTTCACTGC (SEQ ID NO: 24) | |
| SS_F_v2 | ACACGACGCTCTTCCGATCT ACCGTGGTGCC (SEQ ID NO: | Offset PCR v2 |
| | 25) | |
| ES_F1_v2 | ACACGACGCTCTTCCGATCT C ACCGTGGTGCC (SEQ ID | Offset PCR v2 |
| | NO: 26) | |
| ES_F2_v2 | ACACGACGCTCTTCCGATCT TA ACCGTGGTGCC (SEQ ID | Offset PCR v2 |
| | NO: 27) | |
| ES_F3_v2 | ACACGACGCTCTTCCGATCT GGT ACCGTGGTGCC (SEQ ID | Offset PCR v2 |
| | NO: 28) | |
| ES_F4_v2 | ACACGACGCTCTTCCGATCT CTAT ACCGTGGTGCC (SEQ | Offset PCR v2 |
| | ID NO: 29) | |
| ES_F5_v2 | ACACGACGCTCTTCCGATCT ACGTA ACCGTGGTGCC (SEQ | Offset PCR v2 |
| | ID NO: 30) | |
| ES_F6_v2 | ACACGACGCTCTTCCGATCT GTACTT ACCGTGGTGCC | Offset PCR v2 |
| | (SEQ ID NO: 31) | |
| SS_R_v2 | AGACGTGTGCTCTTCCGATCT TCAGCCTTCACTGC (SEQ ID | Offset PCR v2 |
| | NO: 32) | |
| ES_R1_v2 | AGACGTGTGCTCTTCCGATCT T TCAGCCTTCACTGC (SEQ | Offset PCR v2 |
| | ID NO: 33) | |
| ES_R2_v2 | AGACGTGTGCTCTTCCGATCT AT TCAGCCTTCACTGC (SEQ | Offset PCR v2 |
| | ID NO: 34) | |
| ES_R3_v2 | AGACGTGTGCTCTTCCGATCT CAA TCAGCCTTCACTGC | Offset PCR v2 |
| | (SEQ ID NO: 35) | |
| ES_R4_v2 | AGACGTGTGCTCTTCCGATCT GCGT TCAGCCTTCACTGC | Offset PCR v2 |
| | (SEQ ID NO: 36) | |
| ES_R5_v2 | AGACGTGTGCTCTTCCGATCT AGCAT TCAGCCTTCACTGC | Offset PCR v2 |
| | (SEQ ID NO: 37) | |
| ES_R6_v2 | AGACGTGTGCTCTTCCGATCT CATGCT | Offset PCR v2 |
| | TCAGCCTTCACTGC (SEQ ID NO: 38) | |
| PhiX_v1 | ACACGACGCTCTTCCGATCT-N₇GAAAAA-N₂₆-TTTTTGTTTT | Customer PhiX v1 |
| | TG-N₂₆- GCACTCAAGN₇-AGATCGGAAGAGCACACGTCT | |
| | (SEQ ID NO: 39) | |
| PhiX_v2 | ACACGACGCTCTTCCGATCT-N₅GAAAAA-N₂₆-TTTTTGTTTT | Customer PhiX v2 |
| | TG-N₂₆-GCCCTCAAGN5-AGATCGGAAGAGCACACGTCT | |
| | (SEQ ID NO: 40) | |

TABLE 2
The RNA aptamer sequence of the present invention and the control group sequence
(wherein fC and fU represent the nucleotide, i.e. C and U are fluorinated,
such as 2′-F-CTP and 2′-F-UTP))

| Name | Sequence (5′end→3′end) | Order |
|---|---|---|
| RLB1 | GGAGCUCAGCCUUCACUGCACGGAAAUUCCACAAGGAAAAUCCGACUGCU | RL_A28-B1-C4 |
| | UCGGCAAUUCAAGUCUUGAAUUACUGUUUGUCGGGCACCACGGUCGGAUC | |
| | CAC (SEQ ID NO: 41) | |
| RLB2 | GGAGCUCAGCCUUCACUGCUAGCUGCGGAUUCAAGUCUUGACAUACUGUU | RL_A13-B2-C19 |
| | CGGCAGCAAACAGUAGGAGGUUAAACGAUCUUGGCACCACGGUCGGAUCC | |
| | AC (SEQ ID NO: 42) | |
| RLB3 | GGAGCUCAGCCUUCACUGCGACGUUUGAAAACGUCUAACACGAGUCUGCU | RL_A36-B3-C309 |
| | UCGGCAGAGUCUGACGGUAUCCCGGCGGAUGUUGGCACCACGGUCGGAUC | |
| | CAC (SEQ ID NO: 43) | |
| RLB4 | GGAGCUCAGCCUUCACUGCUUGGUACACUGUUAAGGAUAUCUCUACUGCU | RL_A49-B4-C392 |
| | UCGGCAGACGGUUUGAAAACCGUUAAUACAGUUGGCACCACGGUCGGAUC | |
| | CAC (SEQ ID NO: 44) | |
| RLB5 | GGAGCUCAGCCUUCACUGCGUGGCUUCGUUAUGACAUCGAUAUAUCUGCU | RL_A54-B5-C42 |
| | UCGGCAGGCCUAGGUGCCUUUCUCGAUGCUUGGGGCACCACGGUCGGAUC | |
| | CAC (SEQ ID NO: 45) | |
| RLB6 | GGAGCUCAGCCUUCACUGCGUCGUCGAUUCAAGUCUUGACUUACUGUUCG | RL_A70-B6-C1 |
| | GCAGAACUGCAUGUGAAAAACAGUUCCCGCGGCACCACGGUCGGAUCCAC | |
| | (SEQ ID NO: 46) | |
| RLB7 | GGAGCUCAGCCUUCACUGCGUUCUAUCGGUAAUACAGUUUGAAAACUGCU | RL_A136-B7-C484 |
| | UCGCAGUCGUAGCACGACCCAACGCUUGCUCCGGCACCACGGUCGGAUCC | |
| | AC (SEQ ID NO: 47) | |
| RLB8 | GGAGCUCAGCCUUCACUGCCGAGCAUUGCAAGUCUUGACUUACUGCUGCU | RL_A66-B8-C13 |
| | UCGGCAGUCCGUAGUGUUGCCUAUGGUCGCCAGGGCACCACGGUCGGAUC | |
| | CAC (SEQ ID NO: 48) | |
| RLB9 | GGAGCUCAGCCUUCACUGCAGGAUAUUACGCUUGACUACGUGUUCCUGCU | RL_A4-B9-C11 |
| | UCGGCAGUGUGUGACGAGCACUGACGACCUCUCGGCACCACGGUCGGAUC | |
| | CAC (SEQ ID NO: 49) | |
| RLB10 | GGAGCUCAGCCUUCACUGCAGCGAGAUACUCUUGAUAAAGUCCGUCUGCU | RL_A77-B10-C10 |
| | UCGGCAGGUUAGUAGCUUAUGCCGUUGUGUCGUGGCACCACGGUCGGAUC | |
| | CAC (SEQ ID NO: 50) | |
| RLB11 | GGAGCUCAGCCUUCACUGCACUACCCAAUGUUACGCUUGACUACGUGCUU | RL_A97-B11-C16 |
| | CGGCAGUCCAUGGAGGCAUUAACCACCGUUGAGGCACCACGGUCGGAUCC | |
| | AC (SEQ ID NO: 51) | |
| RLB12 | GGAGCUCAGCCUUCACUGCUGACCGAUUCAAUGCAGUGAACGGCACUGCU | RL_A12-B12-C206 |
| | UCGGCAGUCCGGGUGUCGUGGAUAGGCUAUAACGGCACCACGGUCGGAUC | |
| | CAC (SEQ ID NO: 52) | |
| RLB13 | GGAGCUCAGCCUUCACUGCUGGCUCGAUCGUCCAUAGCUCUAGAGCUGCU | RL_A8-B13-C3 |
| | UCGGCAAAGGUCCUCUUGAUAAAGUCAAGCCGAGGCACCACGGUCGGAUC | |
| | CAC (SEQ ID NO: 53) | |
| RLB14 | GGAGCUCAGCCUUCACUGCCAGGCUAGGCUUGGCCCCAUUUUUACCUGCU | RL_A1-B14-C2 |
| | UCGGCAGGACGCCCGUGUGUGAAUAUAAACCCUGGCACCACGGUCGGAUC | |
| | CAC (SEQ ID NO: 54) | |
| RLB15 | GGAGCUCAGCCUUCACUGCAGUAAUGUUGAAACAGGACGUCUUCUGCUUC | RL_A112-B15-C449 |
| | GGCAGAGAUUAGGUUAUCACCCUGUGGGGAAGGCACCACGGUCGGAUCCA | |
| | C (SEQ ID NO: 55) | |
| RLB16 | GGAGCUCAGCCUUCACUGCAUGGAAGCUGGACUCGUACCGUUUGCUGCUU | RL_A2-B16-C29 |
| | CGGCAGGUAUCGUCUACGUGCUAGCUUGGCUAGGCACCACGGUCGGAUCC | |
| | AC (SEQ ID NO: 56) | |
| RLB17 | GGAGCUCAGCCUUCACUGCGCGCGCAGUACCUGCCACUUGGGGAACUGCU | RL_A61-B17-C59 |
| | UCGGCAGUUGUGCGCGAAGUCCUGGCCGCGGUUGGCACCACGGUCGGAUC | |
| | CAC (SEQ ID NO: 57) | |
| RLB18 | GGAGCUCAGCCUUCACUGCCUCGGUCGAAAGUAAGUCUUGACAUACUGCU | RL_A25-B18-C6 |
| | UCGGCAGAGGCGACGCUUGACCGUGAACACUAAGGCACCACGGUCGGAUC | |
| | CAC (SEQ ID NO: 58) | |
| RLB19 | GGAGCUCAGCCUUCACUGCUUGGCCGAACACAGAUCCAUCUGAACCUGCC | RL_A86-B19-C15 |
| | UCGGCAGCUCUUUCACUAAUCGACGACCCGUUGGCACCACGGUCGGAUCC | |
| | AC (SEQ ID NO: 59) | |
| RLB20 | GGAGCUCAGCCUUCACUGCAUUCAACGAAUUCAAGUCUUGAUAUACUGUU | RL_A32-B20-C7 |
| | UCGGCAGUCGUCCGGUCACUCGGAUGUAUAGCUGGCACCACGGUCGGAUC | |
| | CAC (SEQ ID NO: 60) | |
| RLB21 | GGAGCUCAGCCUUCACUGCGCAGAGGUCCGCUUGAAAACGUCCUGCUGCU | RL_A35-B21-C67 |
| | UCGGCAGCUCCCUCACCGUGGUCGUGCACUGCCGGCACCACGGUCGGAUC | |
| | CAC (SEQ ID NO: 61) | |
| RLB22 | GGAGCUCAGCCUUCACUGCAUCGUGGCGCGUGACUCGUGACAACUCUGCU | RL_A7-B22-C35 |
| | UCGGCAGGUAGUGCGAGGUUGGCCUUGAGCCUUGGCACCACGGUCGGAUC | |
| | CAC (SEQ ID NO: 62) | |
| RLB23 | GGAGCUCAGCCUUCACUGCGAGUGGACCAAGUCUUGACUUACUGGCUGCU | RL_A5-B23-C9 |
| | UGGCAGUGAGACUUAUGUGAGCCUUAACCGUGGGCACCACGGUCGGAUCC | |
| | AC (SEQ ID NO: 63) | |
| RLB24 | GGAGCUCAGCCUUCACUGCUGACUGCUCGAAUGGCCGUGAUGCGACUGCU | RL_A273-B24-C14 |
| | UCGGCAGUCUGGGUUGCGUGCUCCCGCUUGUCGGGCACCACGGUCGGAUC | |
| | CAC (SEQ ID NO: 64) | |
| RLB25 | GGAGCUCAGCCUUCACUGCUAGGUCCGCUUGAUAACGUCAGCAGUCUGCU | RL_A126-B25-C27 |
| | UCGGCAGGUGGUUGAGAUCUGUCGCGAGCCCUUGGCACCACGGUCGGAUC | |
| | CAC (SEQ ID NO: 65) | |
| RLB26 | GGAGCUCAGCCUUCACUGCUCCAUGGCCUUUUCCCUUGAGUAGCUCUGCU | RL_A9-B26-C40 |
| | UCGGCAGAUAUUGUAUCUUGAACACCUACCCAAGGCACCACGGUCGGAUC | |
| | CAC (SEQ ID NO: 66) | |
| RLB29 | GGAGCUCAGCCUUCACUGCUGAGGUGUGUUAUGCUUGACUACAUGCCGCU | RL_A11-B29-C21 |
| | UCGGCAGUCGGUUCGGUCCUCCAUUUUGCGUGCGGCACCACGGUCGGAUC | |
| | CAC (SEQ ID NO: 67) | |
| RLB30 | GGAGCUCAGCCUUCACUGCGAUCAGGUCCGCUUGAUAACGUCGAUCUGCU | RL_A10-B30-C69 |
| | UCUGAAGGAUGAUCCUUACUCUCUCUUUUGAUCGGCACCACGGUCGGAUC | |
| | CAC (SEQ ID NO: 68) | |
| RLB31 | GGAGCUCAGCCUUCACUGCCCGGUGACGGUAAGACUGACUCCUCACUGCU | RL_A337-B31-C25 |
| | UCGGCAGGCUGCUGGUAGCACCUCUUGACAAAGGGCACCACGGUCGGAUC | |
| | CAC (SEQ ID NO: 69) | |
| RLB32 | GGAGCUCAGCCUUCACUGCACUCACGAAUAGCAAGUCUUGAUAUACUGCU | RL_A14-B32-C86 |
| | UCGGCAGCGCAGGCGAAAAGCACCCGUACCGCAGGCACCACGGUCGGAUC | |
| | CAC (SEQ ID NO: 70) | |
| RLB33 | GGAGCUCAGCCUUCACUGCGGAAAGUCCAGGAAUCGCAUUAACCUCUGCU | RL_A16-B33-C71 |
| | UCGGCAGAUGUGUGUGUCAACUCGUUUGGCCUGGCACCACGGUCGGAUCC | |
| | AC (SEQ ID NO: 71) | |
| RLB36 | GGAGCUCAGCCUUCACUGCUAGCUGCGGAUUCAAGUCUUGACAUACUGUU | RL_A20-B36-C23 |
| | CGGCAGCAAAACAGUAGGAGGUUAAACGAUCUUGGCACCACGGUCGGAUC | |
| | CAC (SEQ ID NO: 72) | |
| RLB37 | GGAGCUCAGCCUUCACUGCUAAUAGCAUGGUCCGCUUGACUACGUCUGCU | RL_A179-B37-C22 |
| | UCGGCAGUGGCUGAUUUACCUGUGAUCGUGCGGGCACCACGGUCGGAUCC | |
| | AC (SEQ ID NO: 73) | |
| RLB41 | GGAGCUCAGCCUUCACUGCUCCACCUGUGGUGACCUGUCCUCUGCUUCGG | RL_A84-B41-C20 |
| | CAGAGUGACUUCAAAGCGCUUGAAAACGAGGCACCACGGUCGGAUCCAC | |
| | (SEQ ID NO: 74) | |
| RLB42 | GGAGCUCAGCCUUCACUGCGUCGUCGAUUCAAGUCUUGACUUACUGUUCG | RL_A19-B42-C113 |
| | GCAGAACUGCAUGUGAAAAAACAGUUCCCGCGGCACCACGGUCGGAUCCA | |
| | C (SEQ ID NO: 75) | |
| RLB44 | GGAGCUCAGCCUUCACUGCAGUAGGUAUCCUGAGCCUCAGAUCGUGCUGC | RL_A198-B44-C17 |
| | UUCGGCAGAUCGUGCACCAAGUCUUGAAUUACUGGGCACCACGGUCGGAU | |
| | CCAC (SEQ ID NO: 76) | |
| RLB45 | GGAGCUCAGCCUUCACUGCGGUAGUCGUUUAGCGUGAUGGUUAUGCCGCU | RL_A15-B45-C111 |
| | UCGGCAGUGGUUGCACUUACGCUUGAAUACGUGGGCACCACGGUCGGAUC | |
| | CAC (SEQ ID NO: 77) | |
| RLB50 | GGAGCUCAGCCUUCACUGCACUAGACCUAUGCCGAUGUAAGUACUCUGCU | RL_A3-B50-C5 |
| | UCGGCAGUCCUUUCAGAGUCUUGAGGACUACCCGGCACCACGGUCGGAUC | |
| | CAC (SEQ ID NO: 78) | |
| RLB51 | GGAGCUCAGCCUUCACUGCAUGUUACGCUUGACUACGUGCUGCAGCUGCU | RL_A23-B51-C99 |
| | UCGGCAGUUCCAAUCGUGUGGACUGAGCAAGUAGGCACCACGGUCGGAUC | |
| | CAC (SEQ ID NO: 79) | |
| RLB54 | GGAGCUCAGCCUUCACUGCUUUGCAAGCUUCCGCUUGGCAACGAGCUGCU | RL_A225-B54-C24 |
| | CGGCAGUGCAGCCUCUUCUGCUCUGACCGUCUGGCACCACGGUCGGAUCC | |
| | AC (SEQ ID NO: 80) | |
| RLB63 | GGAGCUCAGCCUUCACUGCAUUCAAGUCUUGAACUACUGUUGCAGCUGCU | RL_A24-B63-C143 |
| | UCGGCAGAAGGGUUCUUUGGUUCAACCCGCGGAGGCACCACGGUCGGAUC | |
| | CAC (SEQ ID NO: 81) | |
| RLB70 | GGAGCUCAGCCUUCACUGCCCUCUCACCGAUACGCUUUUCACCCGCUGCU | RL_A6-B70-C12 |
| | UCGGCGCCAUAGCAAGUCUUGACUUACUGCGUGGCACCACGGUCGGAUCC | |
| | AC (SEQ ID NO: 82) | |
| RLB82 | GGAGCUCAGCCUUCACUGCGACUGAUUUGGAGCCUAAUGUAUAGUCUGCU | RL_A57-B82-C8 |
| | UGGCAGACUUAUUCAGCGUUACCGUACAUUGUGGCACCACGGUCGGAUCC | |
| | AC (SEQ ID NO: 83) | |
| RLB95 | GGAGCUCAGCCUUCACUGCUGCGUCGAAUCGCAAGUCUUGACCUACUGCU | RL_A18-B95-C18 |
| | UCGACAGUACAGGGAGCUGUUCCGCUGCGCCGUGGCACCACGGUCGGAUC | |
| | CAC (SEQ ID NO: 84) | |
| RLB117 | GGAGCUCAGCCUUCACUGCAUUGCCGAAUCCAAGUCUUGAAUUACUGCUU | RL_A17-B117-C41 |
| | CGGCAGCGCUAUCUAGCUCCACCGUUGAACUUGGCACCACGGUCGGAUCC | |
| | AC (SEQ ID NO: 85) | |
| RLB118 | GGAGCUCAGCCUUCACUGCCAAUGUAUGGUCCGCUUGACAACGUCUGCUU | RL_A22-B118-C53 |
| | CGGCAGUGAACUCCCACCACCGCGCACCCUGGGGCACCACGGUCGGAUCC | |
| | AC (SEQ ID NO: 86) | |
| RLB136 | GGAGCUCAGCCUUCACUGCAAGUCUUGACUUACUGCGUGGAGAUGCUGCU | RL_A21-B136-C124 |
| | UCGGCAGAGACGCGGUAAUGACCGACCAUCGUUGGCACCACGGUCGGAUC | |
| | CAC (SEQ ID NO: 87) | |
| NLB1 | GGAGCUCAGCCUUCACUGCGUGGUGUAUAGUUCCUGCGAUGGCAUCUGCU | NL_A2-B1-C1 |
| | UCGGCAGAUAUACUGGGAUCCGUGACGAUCAUCGGCACCACGGUCGGAUC | |
| | CAC (SEQ ID NO: 88) | |
| NLB2 | GGAGCUCAGCCUUCACUGCUGAGGUGUGUUAUGCUUGACUACAUGCCGCU | NL_A4-B2-C2 |
| | UCGGCAGUCGGUUCGGUCCUCCAUUUUGCGUGCGGCACCACGGUCGGAUC | |
| | CAC (SEQ ID NO: 89) | |
| NLB3 | GGAGCUCAGCCUUCACUGCGUGGCUUCGUUAUGACAUCGAUAUAUCUGCU | NL_A3-B3-C3 |
| | UCGGCAGGCCUAGGUGCCUUUCUCGAUGCUUGGGGCACCACGGUCGGAUC | |
| | CAC (SEQ ID NO: 90) | |
| NLB4 | GGAGCUCAGCCUUCACUGCGUGGUGUAUAGCUCCUGCGAUGGCAUCUGCU | NL_A1-B4-C4 |
| | UCGGCAGAUAUCCUGGGAUCCGUGACGAUCAUCGGCACCACGGUCGGAUC | |
| | CAC (SEQ ID NO: 91) | |
| NLB5 | GGAGCUCAGCCUUCACUGCAUUCAAGUCUUGAACUACUGUUGCAGCUGCU | NL_A5-B5-C5 |
| | UCGGCAGAAGGGUUCUUUGGUUCAACCCGCGGAGGCACCACGGUCGGAUC | |
| | CAC (SEQ ID NO: 92) | |
| NLB6 | GGAGCUCAGCCUUCACUGCACGGAAAUUCCACAAGGAAAAUCCGACUGCU | NL_A7-B6-C6 |
| | UCGGCAAUUCAAGUCUUGAAUUACUGUUUGUCGGGCACCACGGUCGGAUC | |
| | CAC (SEQ ID NO: 93) | |
| NLB7 | GGAGCUCAGCCUUCACUGCAUCGCCGAAAAGCAAGUCUUGAAUUACUACU | NL_A34-B7-C9 |
| | UCGGCAGACCGUACCUGUAUCCGGUCUAAGUGUGGCACCACGGUCGGAUC | |
| | CAC (SEQ ID NO: 94) | |
| NLB8 | GGAGCUCAGCCUUCACUGCAGGAUAUUACGCUUGACUACGUGUUCCUGCU | NL_A10-B8-C7 |
| | UCGGCAGUGUGUGACGAGCACUGACGACCUCUCGGCACCACGGUCGGAUC | |
| | CAC (SEQ ID NO: 95) | |
| NLB9 | GGAGCUCAGCCUUCACUGCAGUAGGUAUCCUGAGCCUCAGAUCGUGCUGC | NL_A20-B9-C23 |
| | UUCGGCAGAUCGUGCACCAAGUCUUGAAUUACUGGGCACCACGGUCGGAU | |
| | CCAC (SEQ ID NO: 96) | |
| NLB10 | GGAGCUCAGCCUUCACUGCUGAGGUGUGUUAUGCUUGACUACAUGCCGCU | NL_A42-B10-C8 |
| | UCGGCAGUCGGUUCGGUCCUCCAUUUUGCGUGUGGCACCACGGUCGGAUC | |
| | CAC (SEQ ID NO: 97) | |
| NLB11 | GGAGCUCAGCCUUCACUGCGACGUUUGAAAACGUCUAACACGAGUCUGCU | NL_A23-B11-C15 |
| | UCGGCAGAGUCUGACGGUAUCCCGGCGGAUGUUGGCACCACGGUCGGAUC | |
| | CAC (SEQ ID NO: 98) | |
| NLB12 | GGAGCUCAGCCUUCACUGCGUGGCGUAUAGCUCCUGCGAUGGCAUCUGCU | NL_A25-B12-C14 |
| | UCGGCAGAUAUACUGGGAUCCGUGACGAUCAUCGGCACCACGGUCGGAUC | |
| | CAC (SEQ ID NO: 99) | |
| NLB13 | GGAGCUCAGCCUUCACUGCGUGUGCUCUUCCGAUCUUUCAGCCUUCACUG | NL_A41-B13-C27 |
| | CUGCGGAAAUCGGCUUGGCGUUGAUCAUAUCCUGUGGCACCACGGUCGGA | |
| | UCCAC (SEQ ID NO: 100) | |
| NLB14 | GGAGCUCAGCCUUCACUGCAGGUGGGCAGUUAGCAUUGGCUAAUGCUCCU | NL_A26-B14-C16 |
| | UCGGCAGACGUUGUGACCUAAGCUUGACAUCUUGGCACCACGGUCGGAUC | |
| | CAC (SEQ ID NO: 101) | |
| NLB15 | GGAGCUCAGCCUUCACUGCGUGAUGUAUAGCCCCAGUGAACUAUCCUGCU | NL_A9-B15-C19 |
| | UCGGCAGACAUAUGCUCCGGUCCGCCGGGCAUCGGCACCACGGUCGGAUC | |
| | CAC (SEQ ID NO: 102) | |
| NLB16 | GGAGCUCAGCCUUCACUGCUCAGAAACAGGUCCGCUUGAAUACGUCUGUU | NL_A11-B16-C11 |
| | UCGGCAGGGUAACCGCGGGCUACCACUCGUGUUGGCACCACGGUCGGAUC | |
| | CAC (SEQ ID NO: 103) | |
| NLB17 | GGAGCUCAGCCUUCACUGCAGCUGGCGUGGUGUAUAGUCUCCUGGCUGCU | NL_A8-B17-C18 |
| | UCGACAGCUGUUUAAAUCGAUCUGGCGGACAUUGGCACCACGGUCGGAUC | |
| | CAC (SEQ ID NO: 104) | |
| NLB18 | GGAGCUCAGCCUUCACUGCGUGCUCUUCCGAUCUGCGUUCAGCCUUCACU | NL_A16-B18-C30 |
| | GCUGCGGAAAUCGGCUUGGCGUUGAUCAUAUCCUGUGGCACCACGGUCGG | |
| | AUCCAC (SEQ ID NO: 105) | |
| NLB19 | GGAGCUCAGCCUUCACUGCGUCGUCGAUUCAAGUCUUGACUUACUGUUCG | NL_A14-B19-C10 |
| | GCAGAACUGCAUGUGAAAAACAGUUCCCGCGGCACCACGGUCGGAUCCAC | |
| | (SEQ ID NO: 106) | |
| NLB20 | GGAGCUCAGCCUUCACUGCGUUCUAUCGGUAAUACAGUUUGAAAACUGCU | NL_A39-B20-C20 |
| | UCGCAGUCGUAGCACGACCCAACGCUUGCUCCGGCACCACGGUCGGAUCC | |
| | AC (SEQ ID NO: 107) | |
| NLB21 | GGAGCUCAGCCUUCACUGCUAGCUGCGGAUUCAAGUCUUGACAUACUGUU | NL_A6-B21-C13 |
| | CGGCAGCAAACAGUAGGAGGUUAAACGAUCUUGGCACCACGGUCGGAUCC | |
| | AC (SEQ ID NO: 108) | |
| NLB22 | GGAGCUCAGCCUUCACUGCACUAGACCUAUGCCGAUGUAAGUACUCUGCU | NL_A13-B22-C17 |
| | UCGGCAGUCCUUUCAGAGUCUUGAGGACUACCCGGCACCACGGUCGGAUC | |
| | CAC (SEQ ID NO: 109) | |
| NLB23 | GGAGCUCAGCCUUCACUGCGCGCGCAGUACCUGCCACUUGGGGAACUGCU | NL_A12-B23-C34 |
| | UCGGCAGUUGUGCGCGAAGUCCUGGCCGCGGUUGGCACCACGGUCGGAUC | |
| | CAC (SEQ ID NO: 110) | |
| NLB24 | GGAGCUCAGCCUUCACUGCGUGGUGUAUAGCUCCUGCGAUGGCCAGCUUC | NL_A21-B24-C43 |
| | GGCUGAUACUGGGAUCCGUGACGAUCAUCGGCACCACGGUCGGAUCCAC | |
| | (SEQ ID NO: 111) | |
| NLB25 | GGAGCUCAGCCUUCACUGCUCUUCCGAUCUAGCAUUCAGCCUUCACUGCU | NL_A29-B25-C24 |
| | GCGGAAAUCGGCUUGGCGUUGAUCAUAUCCUGUGGCACCACGGUCGGAUC | |
| | CAC (SEQ ID NO: 112) | |
| NLB26 | GGAGCUCAGCCUUCACUGCACGUGUGCUCUUCCGAUCUUCAGCCUUCACU | NL_A50-B26-C47 |
| | GCUGCGGAAAUCGGCUUGGCGUUGAUCAUAUCCUGUGGCACCACGGUCGG | |
| | AUCCAC (SEQ ID NO: 113) | |
| NLB27 | GGAGCUCAGCCUUCACUGCAAGAUGUGGACCAUUUAACUUGUAGACUGCU | NL_A17-B27-C12 |
| | UCGGCAGGCGGCUGUUCCCUCAAGGGAACGCUUGGCACCACGGUCGGAUC | |
| | CAC (SEQ ID NO: 114) | |
| NLB28 | GGAGCUCAGCCUUCACUGCACUACCCAAUGUUACGCUUGACUACGUGCUU | NL_A22-B28-C21 |
| | CGGCAGUCCAUGGAGGCAUUAACCACCGUUGAGGCACCACGGUCGGAUCC | |
| | AC (SEQ ID NO: 115) | |
| NLB29 | GGAGCUCAGCCUUCACUGCUCUAGCACGUUAAGCUUGACUACUUGCUGCU | NL_A32-B29-C53 |
| | UCGGCAGAUGUUGCUGCAUUACUACCGAUUGUUGGCACCACGGUCGGAUC | |
| | CAC (SEQ ID NO: 116) | |
| NLB30 | GGAGCUCAGCCUUCACUGCCAAUGUAUGGUCCGCUUGACAACGUCUGCUU | NL_A15-B30-C22 |
| | CGGCAGUGAACUCCCACCACCGCGCACCCUGGGGCACCACGGUCGGAUCC | |
| | AC (SEQ ID NO: 117) | |
| NLB31 | GGAGCUCAGCCUUCACUGCUGAGUGUGUUAUGCUUGACUACAUGCCGCUU | NL_A86-B31-C46 |
| | CGGCAGUCGGUUCGGUCCUCCAUUUUGCGUGCGGCACCACGGUCGGAUCC | |
| | AC (SEQ ID NO: 118) | |
| NLB32 | GGAGCUCAGCCUUCACUGCUAGGUCCGCUUGAUAACGUCAGCAGUCUGCU | NL_A83-B32-C44 |
| | UCGGCAGGUGGUUGAGAUCUGUCGCGAGCCCUUGGCACCACGGUCGGAUC | |
| | CAC (SEQ ID NO: 119) | |
| NLB33 | GGAGCUCAGCCUUCACUGCUUUGCAAGCUUCCGCUUGGCAACGAGCUGCU | NL_A49-B33-C26 |
| | CGGCAGUGCAGCCUCUUCUGCUCUGACCGUCUGGCACCACGGUCGGAUCC | |
| | AC (SEQ ID NO: 120) | |
| NLB34 | GGAGCUCAGCCUUCACUGCUGGCUCGAUCGUCCAUAGCUCUAGAGCUGCU | NL_A30-B34-C29 |
| | UCGGCAAAGGUCCUCUUGAUAAAGUCAAGCCGAGGCACCACGGUCGGAUC | |
| | CAC (SEQ ID NO: 121) | |
| NLB35 | GGAGCUCAGCCUUCACUGCAGUAAUGUUGAAACAGGACGUCUUCUGCUUC | NL_A76-B35-C37 |
| | GGCAGAGAUUAGGUUAUCACCCUGUGGGGAAGGCACCACGGUCGGAUCCA | |
| | C (SEQ ID NO: 122) | |
| NLB36 | GGAGCUCAGCCUUCACUGCAUGGAAGCUGGACUCGUACCGUUUGCUGCUU | NL_A37-B36-C36 |
| | CGGCAGGUAUCGUCUACGUGCUAGCUUGGCUAGGCACCACGGUCGGAUCC | |
| | AC (SEQ ID NO: 123) | |
| NLB37 | GGAGCUCAGCCUUCACUGCUUGGUACACUGUUAAGGAUAUCUCUACUGCU | NL_A118-B37-C50 |
| | UCGGCAGACGGUUUGAAAACCGUUAAUACAGUUGGCACCACGGUCGGAUC | |
| | CAC (SEQ ID NO: 124) | |
| NLB38 | GGAGCUCAGCCUUCACUGCAGCGAGAUACUCUUGAUAAAGUCCGUCUGCU | NL_A19-B38-C28 |
| | UCGGCAGGUUAGUAGCUUAUGCCGUUGUGUCGUGGCACCACGGUCGGAUC | |
| | CAC (SEQ ID NO: 125) | |
| NLB39 | GGAGCUCAGCCUUCACUGCAUGUUACGCUUGACUACGUGCUGCAGCUGCU | NL_A24-B39-C42 |
| | UCGGCAGUUCCAAUCGUGUGGACUGAGCAAGUAGGCACCACGGUCGGAUC | |
| | CAC (SEQ ID NO: 126) | |
| NLB40 | GGAGCUCAGCCUUCACUGCCUCGGUCGAAAGUAAGUCUUGACAUACUGCU | NL_A27-B40-C52 |
| | UCGGCAGAGGCGACGCUUGACCGUGAACACUAAGGCACCACGGUCGGAUC | |
| | CAC (SEQ ID NO: 127) | |
| NLB41 | GGAGCUCAGCCUUCACUGCAAGUCUUGAUAUACUGCUGUCGGCUACUGCU | NL_A18-B41-C25 |
| | UCGGCAGCUAACCGGAGUCCAUUGACGUCGAUGGGCACCACGGUCGGAUC | |
| | CAC (SEQ ID NO: 128) | |
| NLB42 | GGAGCUCAGCCUUCACUGCUUAGUGCCGUCGAGUUAUCCUCAUAACUGCU | NL_A38-B42-C33 |
| | UGGCAGUAUCCUCGCACAUAAGUGCGUUGGUCGGCACCACGGUCGGAUCC | |
| | AC (SEQ ID NO: 129) | |
| NLB43 | GGAGCUCAGCCUUCACUGCCGAGCAUUGCAAGUCUUGACUUACUGCUGCU | NL_A48-B43-C32 |
| | UCGGCAGUCCGUAGUGUUGCCUAUGGUCGCCAGGGCACCACGGUCGGAUC | |
| | CAC (SEQ ID NO: 130) | |
| NLB44 | GGAGCUCAGCCUUCACUGCUUCCGAACAGGGCGAAGCGAAUCCGACUGCU | NL_A28-B44-C31 |
| | UCGACAGCUCAAGUCUUGAUUUACUGUCUGUCUGGCACCACGGUCGGAUC | |
| | CAC (SEQ ID NO: 131) | |
| NLB45 | GGAGCUCAGCCUUCACUGCAUGAGGUCCGCUUGAUAACGUCCAUGCUGCU | NL_A43-B45-C58 |
| | UCGGCAGAGUUCGGCAGGGUAUCUAUGUGCCCUGGCACCACGGUCGGAUC | |
| | CAC (SEQ ID NO: 132) | |
| NLB46 | GGAGCUCAGCCUUCACUGCUGACCGAUUCAAUGCAGUGAACGGCACUGCU | NL_A44-B46-C40 |
| | UCGGCAGUCCGGGUGUCGUGGAUAGGCUAUAACGGCACCACGGUCGGAUC | |
| | CAC (SEQ ID NO: 133) | |
| NLB47 | GGAGCUCAGCCUUCACUGCCAGGCUAGGCUUGGCCCCAUUUUUACCUGCU | NL_A82-B47-C35 |
| | UCGGCAGGACGCCCGUGUGUGAAUAUAAACCCUGGCACCACGGUCGGAUC | |
| | CAC (SEQ ID NO: 134) | |
| NLB48 | GGAGCUCAGCCUUCACUGCCAAUCGUCCUGACCGCCAUUGGGUAGCUGCU | NL_A74-B48-C41 |
| | UCGGCAGAGGCGCCCGUUUCAAUCCACUUGUUGGGCACCACGGUCGGAUC | |
| | CAC (SEQ ID NO: 135) | |
| NLB49 | GGAGCUCAGCCUUCACUGCGUGUGCUCUUCCGAUCUAUUCAGCCUUCACU | NL_A56-B49-C65 |
| | GCUGCGGAAAUCGGCUUGGCGUUGAUCAUAUCCUGUGGCACCACGGUCGG | |
| | AUCCAC (SEQ ID NO: 136) | |
| NLB50 | GGAGCUCAGCCUUCACUGCUGUGCUCUUCCGAUCUCAAUCAGCCUUCACU | NL_A64-B50-C48 |
| | GCUGCGGAAAUCGGCUUGGCGUUGAUCAUAUCCUGUGGCACCACGGUCGG | |
| | AUCCAC (SEQ ID NO: 137) | |
| NLB51 | GGAGCUCAGCCUUCACUGCAGAGGAGUAUACCGAGGCAGCACCCGCUGCU | NL_A45-B51-C70 |
| | UCGGCAGGACAAACUGUAUGGCCCUCGUGCGUUGGCACCACGGUCGGAUC | |
| | CAC (SEQ ID NO: 138) | |
| NLB52 | GGAGCUCAGCCUUCACUGCACAUCACAUUAGUUGACACGUGAAGCCUGCU | NL_A53-B52-C88 |
| | UCGCAGGUGCUUAGGUCCGCUUGAAAACGUCAGGCACCACGGUCGGAUCC | |
| | AC (SEQ ID NO: 139) | |
| NLB53 | GGAGCUCAGCCUUCACUGCUUGGCCGAACACAGAUCCAUCUGAACCUGCC | NL_A54-B53-C39 |
| | UCGGCAGCUCUUUCACUAAUCGACGACCCGUUGGCACCACGGUCGGAUCC | |
| | AC (SEQ ID NO: 140) | |
| NLB54 | GGAGCUCAGCCUUCACUGCAAUUUCCGUUUGAAAACGGGUUAAUACUGCU | NL_A55-B54-C91 |
| | UCGGCAGUAUGUCAGUCUGACAUUGCAGCUCCCGGCACCACGGUCGGAUC | |
| | CAC (SEQ ID NO: 141) | |
| NLB55 | GGAGCUCAGCCUUCACUGCAAGGCCAUGGUCUGCGCUCCCACGUGCUGCU | NL_A81-B55-C59 |
| | UCAGCAGAGCAAGUCUUGACCUACUACCUGUUGGGCACCACGGUCGGAUC | |
| | CAC (SEQ ID NO: 142) | |
| NLB56 | GGAGCUCAGCCUUCACUGCGUGGUGUAUAGCUCCUGCGAUGGCAUCUGCU | NL_A35-B56-C69 |
| | UCGGCAGAUAUACUGGGAUCCGUGACGAUCAUCGGCACCACGGUCGGAUC | |
| | CAC (SEQ ID NO: 143) | |
| NLB57 | GGAGCUCAGCCUUCACUGCGACUGAUUUGGAGCCUAAUGUAUAGUCUGCU | NL_A36-B57-C57 |
| | UGGCAGACUUAUUCAGCGUUACCGUACAUUGUGGCACCACGGUCGGAUCC | |
| | AC (SEQ ID NO: 144) | |
| NLB58 | GGAGCUCAGCCUUCACUGCUGAGGUGUGUUAUGCUUGACUACAUGCCGCU | NL_A77-B58-C60 |
| | UCGGCAGUCGGUUCGGUCCUCCAUUUUGGUGCGGCACCACGGUCGGAUCC | |
| | AC (SEQ ID NO: 145) | |
| NLB59 | GGAGCUCAGCCUUCACUGCAGUUACCAAGUCUUGAUAUACUGGAACUGCU | NL_A172879-B59-C63 |
| | UCGGCAGGUUCGUUUGCCUCCCGUUCGUCGUCAUGGCACCACGGUCGGAU | |
| | CCAC (SEQ ID NO: 146) | |
| NLB60 | GGAGCUCAGCCUUCACUGCCACGAUGGACAGUUUGAAAACUGUUUCUGCU | NL_A88-B60-C61 |
| | UCGGCAGGGAUAUCUCCCUUCGUGCGCGCGUUAGGCACCACGGUCGGAUC | |
| | CAC (SEQ ID NO: 147) | |
| NLB61 | GGAGCUCAGCCUUCACUGCUCCAUGGCCUUUUCCCUUGAGUAGCUCUGCU | NL_A67-B61-C66 |
| | UCGGCAGAUAUUGUAUCUUGAACACCUACCCAAGGCACCACGGUCGGAUC | |
| | CAC (SEQ ID NO: 148) | |
| NLB62 | GGAGCUCAGCCUUCACUGCAGAGGUGCACUUCCGCCAAGGUACUUCUGCU | NL_A51-B62-C73 |
| | UCGGCAGUGUGCGAUGUUCUCUUGACAAAGACAGGCACCACGGUCGGAUC | |
| | CAC (SEQ ID NO: 149) | |
| NLB63 | GGAGCUCAGCCUUCACUGCAUUCAACGAAUUCAAGUCUUGAUAUACUGUU | NL_A58-B63-C38 |
| | UCGGCAGUCGUCCGGUCACUCGGAUGUAUAGCUGGCACCACGGUCGGAUC | |
| | CAC (SEQ ID NO: 150) | |
| NLB65 | GGAGCUCAGCCUUCACUGCGUUGGUAUGUUACUCUUGAAUAAGUGCUGCU | NL_A31-B65-C62 |
| | UCGCAGUGAUUCGCGACUUCUGCCCCUGUCUCGGCACCACGGUCGGAUCC | |
| | AC (SEQ ID NO: 151) | |
| NLB67 | GGAGCUCAGCCUUCACUGCUCUUCCGAUCUCAUGCUUCAGCCUUCACUGC | NL_A33-B67-C45 |
| | UGCGGAAAUCGGCUUGGCGUUGAUCAUAUCCUGUGGCACCACGGUCGGAU | |
| | CCAC (SEQ ID NO: 152) | |
| NLB68 | GGAGCUCAGCCUUCACUGCGAUCAGGUCCGCUUGAUAACGUCGAUCUGCU | NL_A57-B68-C51 |
| | UCUGAAGGAUGAUCCUUACUCUCUCUUUUGAUCGGCACCACGGUCGGAUC | |
| | CAC (SEQ ID NO: 153) | |
| NLB69 | GGAGCUCAGCCUUCACUGCCAACAGAGUUACGCUUGAUGACGUGCCUGCU | NL_A87-B69-C96 |
| | UCGGCAGAUGUCUUGUGGUUAGCUUCAUCCGUGGCACCACGGUCGGAUCC | |
| | AC (SEQ ID NO: 154) | |
| NLB70 | GGAGCUCAGCCUUCACUGCGUGGCUUCGUUAUGACAUCGAUAUAUCUGCU | NL_A169-B70-C64 |
| | UCGGCAGGCCUAGGUGCCUUUCUCGAUGCUUGUGGCACCACGGUCGGAUC | |
| | CAC (SEQ ID NO: 155) | |
| NLB71 | GGAGCUCAGCCUUCACUGCCCUCUCACCGAUACGCUUUUCACCCGCUGCU | NL_A1863-B71-C67 |
| | UCGGCGCCAUAGCAAGUCUUGACUUACUGCGUGGCACCACGGUCGGAUCC | |
| | AC (SEQ ID NO: 156) | |
| NLB73 | GGAGCUCAGCCUUCACUGCUUCCGUGCAGUGUAGCUCCCUGUCAUCUGCU | NL_A59-B73-C94 |
| | UCGGCAGUGCCGUUGCCAGUAUUGCGUCAAACUGGCACCACGGUCGGAUC | |
| | CAC (SEQ ID NO: 157) | |
| NLB76 | GGAGCUCAGCCUUCACUGCAACCACGACCUCCAUAGUGUGCAUUCCUACU | NL_A148-B76-C75 |
| | UCGGCAGUCGGUGUACUAAGUCUUGACAUACUAGGCACCACGGUCGGAUC | |
| | CAC (SEQ ID NO: 158) | |
| NLB79 | GGAGCUCAGCCUUCACUGCAGGCGAGUCCGCUUGAAUACGUUCGUCUGCU | NL_A164-B79-C56 |
| | UCGGCAGCGCCACUUGGCUCUUGGUGCUGUGGGGGCACCACGGUCGGAUC | |
| | CAC (SEQ ID NO: 159) | |
| NLB80 | GGAGCUCAGCCUUCACUGCUAAUAGCAUGGUCCGCUUGACUACGUCUGCU | NL_A46-B80-C79 |
| | UCGGCAGUGGCUGAUUUACCUGUGAUCGUGCGGGCACCACGGUCGGAUCC | |
| | AC (SEQ ID NO: 160) | |
| NLB81 | GGAGCUCAGCCUUCACUGCUGACUGCUCGAAUGGCCGUGAUGCGACUGCU | NL_A94-B81-C87 |
| | UCGGCAGUCUGGGUUGCGUGCUCCCGCUUGUCGGGCACCACGGUCGGAUC | |
| | CAC (SEQ ID NO: 161) | |
| NLB83 | GGAGCUCAGCCUUCACUGCUUGACCGUUGGUAGUUUCGAGCUUCGCUGCU | NL_A65-B83-C49 |
| | UCGGCAGUUAUCGGGUUGUGCGCUCGCCCUUGUGGCACCACGGUCGGAUC | |
| | CAC (SEQ ID NO: 162) | |
| NLB84 | GGAGCUCAGCCUUCACUGCGGUAGUCGUUUAGCGUGAUGGUUAUGCCGCU | NL_A93-B84-C113 |
| | UCGGCAGUGGUUGCACUUACGCUUGAAUACGUGGGCACCACGGUCGGAUC | |
| | CAC (SEQ ID NO: 163) | |
| NLB86 | GGAGCUCAGCCUUCACUGCGGAAAGUCCAGGAAUCGCAUUAACCUCUGCU | NL_A71-B86-C55 |
| | UCGGCAGAUGUGUGUGUCAACUCGUUUGGCCUGGCACCACGGUCGGAUCC | |
| | AC (SEQ ID NO: 164) | |
| NLB90 | GGAGCUCAGCCUUCACUGCAUCGUGGCGCGUGACUCGUGACAACUCUGCU | NL_A63-B90-C54 |
| | UCGGCAGGUAGUGCGAGGUUGGCCUUGAGCCUUGGCACCACGGUCGGAUC | |
| | CAC (SEQ ID NO: 165) | |
| NLB99 | GGAGCUCAGCCUUCACUGCAGCCUCAAAUCUGCGCAAUCCGUGGUCUGCU | NL_A102-B99-C105 |
| | UCGGCAGUCGCUUGACCGUCCCGUAGUUUCCGGGGCACCACGGUCGGAUC | |
| | CAC (SEQ ID NO: 166) | |
| NLB100 | GGAGCUCAGCCUUCACUGCGGUGUACCGUGGUCCGCCGUAAUAUCUGCUU | NL_A40-B100-C74 |
| | CGGCAGCAAGUCUUGACAUACUGCGCCUACACGGCACCACGGUCGGAUCC | |
| | AC (SEQ ID NO: 167) | |
| NLB113 | GGAGCUCAGCCUUCACUGCACGCACCGAAAGUAAGUCUUGACAUACUGCU | NL_A106-B113-C72 |
| | UCGGCAGAAGGCGGACAGCCCAGACCGCGGCGCGGCACCACGGUCGGAUC | |
| | CAC (SEQ ID NO: 168) | |
| NLB137 | GGAGCUCAGCCUUCACUGCUAGCGCCGGAUUCAAGUCUUGAAUUACUGUU | NL_A81163-B137-C123 |
| | UCGGCAGCUAGUGACGUGUGGCCUGCCCUAACGGGCACCACGGUCGGAUC | |
| | CAC (SEQ ID NO: 169) | |
| NLB138 | GGAGCUCAGCCUUCACUGCUAGCUGCGGAUUCAAGUCUUGACAUACUGUU | NL_A52-B138-C90 |
| | CGGCAGCAAAACAGUAGGAGGUUAAACGAUCUUGGCACCACGGUCGGAUC | |
| | CAC (SEQ ID NO: 170) | |
| NLB217 | GGAGCUCAGCCUUCACUGCGCAGAGGUCCGCUUGAAAACGUCCUGCUGCU | NL_A47-B217-C146 |
| | UCGGCAGCUCCCUCACCGUGGUCGUGCACUGCCGGCACCACGGUCGGAUC | |
| | CAC (SEQ ID NO: 171) | |
| non-preL | GGAGCUCAGCCUUCACUGCUACCCCCGCCAGCGUGUCUUGACAUACUGCG | NL_filter-seq |
| | GCGGGGUUUGAUCCUCGAGACCUGUCUUGGCACCACGGUCGGAUCCAC | non-rank |
| | (SEQ ID NO: 172) | |
| RNAI | ACAGUAUUUGGUAUCUGCGCUCUGCUGAAGCCAGUUACCUUCGGAAAAAG | NL_Anan-Bnan-Cnan |
| | AGUUGGUAGCUCUUGAUCCGGCAAACAAACCACCGCUGGUAGCGGUGGUU | |
| | UUUUUU (SEQ ID NO: 173) | |
| Ref-RNA | AGAUCACAGAGAUGUGAUGGAAAAUAGUUGAUGAGUUGUUUAAUUUUAA | NL_Anan-Bnan-Cnan |
| | GAAUUUUUAUCUUAAUUAAGGAAGGAGUGAUUUCAAUGGCACAAGAUAU | |
| | CAUUUCAACAAUCGG (SEQ ID NO: 174) | |
| RT-F1 | GGAGfCfUfCAGfCfCfUfUfCAfCfUGfCAfCGAfUAGfUfCGfCfCfCAfUGGfCfCAfC | RT30F_1 |
| | AfUfUAfUAfCGAGGfCAfCfCAfCGGfUfCGGAfUfCfCAfC (SEQ ID NO: 175) | |
| RT-F2 | GGAGfCfUfCAGfCfCfUfUfCAfCfUGfCAfCfUAfUfUGfUGAfCfCfCfCfUAGAAfCA | RT30F_2 |
| | GfUGGfUfUfUfUfCGGfCAfCfCAfCGGfUfCGGAfUfCfCAfC (SEQ ID NO: 176) | |
| RT-F3 | GGAGfCfUfCAGfCfCfUfUfCAfCfUGfCfUGfUfUGGAGfCAfCfUfUAfCAAAGAfC | RT30F_3 |
| | GfCfCfUAGGGAGGfCAfCfCAfCGGfUfCGGAfUfCfCAfC (SEQ ID NO: 177) | |
| RT-F4 | GGAGfCfUfCAGfCfCfUfUfCAfCfUGfCGAAGAAfCfCGfUfUfCGfCGfUfCfUfCGfC | RT30F_4 |
| | fUfCAAfUGfCGfUGGfCAfCfCAfCGGfUfCGGAfUfCfCAfC (SEQ ID NO: 178) | |
| RT-F5 | GGAGfCfUfCAGfCfCfUfUfCAfCfUGfCAfCAfCfUfUfUAfUGfUfCfCAGGGfCAfUf | RT30F_5 |
| | CfUGAAGGAfUfUGGfCAfCfCAfCGGfUfCGGAfUfCfCAfC (SEQ ID NO: 179) | |
| RT-F6 | GGAGfCfUfCAGfCfCfUfUfCAfCfUGfCGfUfCfUGfUfCAfUGAfUAAfUGGGAfUfC | RT30F_6 |
| | GfUfUAfCGGfCGGGfCAfCfCAfCGGfUfCGGAfUfCfCAfC (SEQ ID NO: 180) | |
| RT-F7 | GGAGfCfUfCAGfCfCfUfUfCAfCfUGfCGGfUGAGAAAfUfCGfUGfUAfCfUfCAfUf | RT30F_7 |
| | CfUAfCfCGfUAAGGfCAfCfCAfCGGfUfCGGAfUfCfCAfC (SEQ ID NO: 181) | |
| RT-F8 | GGAGfCfUfCAGfCfCfUfUfCAfCfUGfCfCfCGfCfCfUfUAfUAAAGGGfUAGfUGAf | RT30F_8 |
| | CAfUfCfUGfUfUAGGfCAfCfCAfCGGfUfCGGAfUfCfCAfC (SEQ ID NO: 182) | |
| RT-F9 | GGAGfCfUfCAGfCfCfUfUfCAfCfUGfCAAfCAAfCfCGGfCfCfUAGfUGfUAGGfUf | RT30F_9 |
| | CfUGGfUfCAfUGGGfCAfCfCAfCGGfUfCGGAfUfCfCAfC (SEQ ID NO: 183) | |
| RT-F10 | GGAGfCfUfCAGfCfCfUfUfCAfCfUGfCfUGfCGAfCfCGGfUfUfCAGfUAAGfCAG | RT30F_10 |
| | AAfUGGGAfCAGGfCAfCfCAfCGGfUfCGGAfUfCfCAfC (SEQ ID NO: 184) | |
| RT-F11 | GGAGfCfUfCAGfCfCfUfUfCAfCfUGfCGAAGfCfUAfUGAfUfCGAGfCAfUAfCGA | RT30F_11 |
| | fCfCfCAGAGAGGfCAfCfCAfCGGfUfCGGAfUfCfCAfC (SEQ ID NO: 185) | |
| RT-F12 | GGAGfCfUfCAGfCfCfUfUfCAfCfUGfCfUfUAGGfUAfCfUAGGfCfUGfUAGfUAA | RT30F_12 |
| | GfUGfCfUAfUGAGGfCAfCfCAfCGGfUfCGGAfUfCfCAfC (SEQ ID NO: 186) | |
| RT-Fcg1 | GGAGfCfUfCAGfCfCfUfUfCAfCfUGfCfUGGAGAfUfUfCfCGGGfUfUfCAfCfUAf | RT30F_16 |
| | UfUAfCAAGGUAGGfCAfCfCAfCGGfUfCGGAfUfCfCAfC (SEQ ID NO: 187) | |
| RT-Fcg2 | GGAGfCfUfCAGfCfCfUfUfCAfCfUGfCGfCfUfUfUfCfUfUGfUfCfCGfUGGfUAAfC | RT30F_34 |
| | GGGfCfCGGfCGfUGGfCAfCfCAfCGGfUfCGGAfUfCfCAfC (SEQ ID NO: 188) | |
| RT-Fcg3 | GGAGfCfUfCAGfCfCfUfUfCAfCfUGfCAGGAfUAfUfUfCfUfCAfCGGGfCfCAfUfU | RT30F_187 |
| | fUfUGAfUfCfCGfUGGfCAfCfCAfCGGfUfCGGAfUfCfCAfC (SEQ ID NO: 189) | |
| RT-Fcg4 | GGAGfCfUfCAGfCfCfUfUfCAfCfUGfCAAfCfUAfCGfUAAfCAGGAfCGAfUfCfCG | RT30F_262 |
| | GGfCfUGAAfUGGfCAfCfCAfCGGfUfCGGAfUfCfCAfC (SEQ ID NO: 190) | |
| RT-FN1 | GGAGfCfUfCAGfCfCfUfUfCAfCfUGfCfUfCfUAAfUAfCGAfCfUfCAfCfUAfUAGG | RT30FN_1 |
| | AGfCfUfCAGfCfCGGfCAfCfCAfCGGfUfCGGAfUfCfCAfC (SEQ ID NO: 191) | |
| RT-FN2(n) | GGAGCUCAGCCUUCACUGCUACCCCCGCCAGCGUGUCUUGGCACCACGGU | RT30FN_2 |
| | CGGAUCCAC (SEQ ID NO: 192) | |
| RT-FN3 | GGAGfCfUfCAGfCfCfUfUfCAfCfUGfCAfUfUfCfUGfUAAAfCAAAfUGGfCAGfC | RT30FN_3 |
| | AGGfUGGAGfUGGfCAfCfCAfCGGfUfCGGAfUfCfCAfC (SEQ ID NO: 193) | |
| RT-FN4(n) | GGAGCUCAGCCUUCACUGCUAUUGACGAGAUUUCUCUUAGGCACCACGGU | RT30FN_4 |
| | CGGAUCCAC (SEQ ID NO: 194) | |
| RT-FN5(n) | GGAGCUCAGCCUUCACUGCUGGUUUAUCCUUCGUUACAAGGCACCACGGU | RT30FN_5 |
| | CGGAUCCAC (SEQ ID NO: 195) | |
| RT-FN6 | GGAGfCfUfCAGfCfCfUfUfCAfCfUGfCGfCfUfUfUfUfUfCfCGGGGAfCfCGfCfCfC | RT30FN_6 |
| | AGGfUfUGfUfUfUfUGGfCAfCfCAfCGGfUfCGGAfUfCfCAfC (SEQ ID NO: 196) | |
| RT-FN7(n) | GGAGCUCAGCCUUCACUGCCGGAACCUUCGGUCAGUCACGGCACCACGGU | RT30FN_7 |
| | CGGAUCCAC (SEQ ID NO: 197) | |
| RT-FN8(n) | GGAGCUCAGCCUUCACUGCCCACGGUCACCGUAAAACUCGGCACCACGGU | RT30FN_8 |
| | CGGAUCCAC (SEQ ID NO: 198) | |
| RT-FN9(n) | GGAGCUCAGCCUUCACUGCGUCAAAGAUACUAUACCGUCGGCACCACGGU | RT30FN_9 |
| | CGGAUCCAC (SEQ ID NO: 199) | |
| RT-FN10(n) | GGAGCUCAGCCUUCACUGCGAGAUGCAAGAACAAGCGUAGGCACCACGGU | RT30FN_10 |
| | CGGAUCCAC (SEQ ID NO: 200) | |
| RT-FN11 | GGAGfCfUfCAGfCfCfUfUfCAfCfUGfCfUfCfUAfCGGfCfCAAAAAGfCAGAfUAA | RT30FN_11 |
| | GGfUfUAfUAGGGfCAfCfCAfCGGfUfCGGAfUfCfCAfC (SEQ ID NO: 201) | |
| RT-FN12 | GGAGfCfUfCAGfCfCfUfUfCAfCfUGfCAAfUGfCfUfUfCfCfCAAfCfCAGAfUfCAf | RT30FN_12 |
| | UfCGAfCfCfUfUfUfUGGfCAfCfCAfCGGfUfCGGAfUfCfCAfC (SEQ ID NO: 202) | |
| RT-FNcgp1 | GGAGfCfUfCAGfCfCfUfUfCAfCfUGfCGGfUGAGGGfCfCGfCAfCAGfUfUfCfCGG | RT30FN_183 |
| | GfUfCfCfUGfCfUGGfCAfCfCAfCGGfUfCGGAfUfCfCAfC (SEQ ID NO: 203) | |
| RT-FNcg2 | GGAGfCfUfCAGfCfCfUfUfCAfCfUGfCGAGAfUGGfUfCfUfCfCGGGAAfUGGfCf | RT30FN_204 |
| | UfUfCAGfUGfUfUGGfCAfCfCAfCGGfUfCGGAfUfCfCAfC (SEQ ID NO: 204) | |
| RT-FNcg3 | GGAGfCfUfCAGfCfCfUfUfCAfCfUGfCAGfUfUfCAAfUGfCAAAAAAfUfCfCGGG | RT30FN_504 |
| | AfCfUfCfUfUAGGGfCAfCfCAfCGGfUfCGGAfUfCfCAfC (SEQ ID NO: 205) | |
| RT-FNcg4 | GGAGfCfUfCAGfCfCfUfUfCAfCfUGfCAAfUGAfUAfCfCAGfCGGfCfUfCfCGfCG | RT30FN_575 |
| | GGAfCfUAfUfCfCGGfCAfCfCAfCGGfUfCGGAfUfCfCAfC (SEQ ID NO: 206) | |
| RT-FF1 | GGAGfCfUfCAGfCfCfUfUfCAfCfUGfCAfCfCGGGAGfCfUfCAGfCfCfUfUfCAfCG | RT30FF_1 |
| | GfCAfCfCAfCGGfUfCGGAfUfCfCAfC (SEQ ID NO: 207) | |
| RT-FF2 | GGAGfCfUfCAGfCfCfUfUfCAfCfUGfCGGfCAfCfCAfCGGfUfCGGAfUfCfCAfCG | RT30FF_2 |
| | GfCAfCfCAfCGGfUfCGGAfUfCfCAfC (SEQ ID NO: 208) | |
| RT-FF3 | GGAGfCfUfCAGfCfCfUfUfCAfCfUGfCAfUAfUGfUGAfUGGfUfUAGfUfUAfUGA | RT30FF_3 |
| | GfUGAfUfCfUAAGGfCAfCfCAfCGGfUfCGGAfUfCfCAfC (SEQ ID NO: 209) | |
| RT-FF4 | GGAGfCfUfCAGfCfCfUfUfCAfCfUGfCAfUfUGfCAAGfCfUAAAfCGfCfCfUfUGfU | RT30FF_4 |
| | AfCAAGfUfUfCGGGfCAfCfCAfCGGfUfCGGAfUfCfCAfC (SEQ ID NO: 210) | |
| RT-FF5 | GGAGfCfUfCAGfCfCfUfUfCAfCfUGfCAfUAGfCGGfCAAGAGGAAfCGfCAAAG | RT30FF_5 |
| | GAfCAfCfCfUGGfCAfCfCAfCGGfUfCGGAfUfCfCAfC (SEQ ID NO: 211) | |
| RT-FF6 | GGAGfCfUfCAGfCfCfUfUfCAfCfUGfCGfCfCGGfCAGfCfUfUfCfCGAfCAfCfUAG | RT30FF_6 |
| | GfCAfCfCAfCGGfUfCGGAfUfCfCAfC (SEQ ID NO: 212) | |
| RT-FF7 | GGAGfCfUfCAGfCfCfUfUfCAfCfUGfCfUfCAAGfCGfCAfCfUfCfCfUfUAGfCAfC | RT30FF_7 |
| | GGfCAfCfCAfCGGfUfCGGAfUfCfCAfC (SEQ ID NO: 213) | |
| RT-FF8 | GGAGfCfUfCAGfCfCfUfUfCAfCfUGfCGAfCfUfUGGfUAfUfUGfUAfUfCAfCAAG | RT30FF_8 |
| | GfCAfCfCAfCGGfUfCGGAfUfCfCAfC (SEQ ID NO: 214) | |
| RT-FF9 | GGAGfCfUfCAGfCfCfUfUfCAfCfUGfCAAfUfCfUGGGAAfUGAfCfUAfCfUAGfU | RT30FF_9 |
| | AfCfUfUfCAAGfUGGfCAfCfCAfCGGfUfCGGAfUfCfCAfC (SEQ ID NO: 215) | |
| RT-FF10 | GGAGfCfUfCAGfCfCfUfUfCAfCfUGfCGfCAAGGGAAAfCAfCfCGGGfCfUGGGf | RT30FF_10 |
| | CAfCfCAfCGGfUfCGGAfUfCfCAfC (SEQ ID NO: 216) | |
| RT-FF11 | GGAGfCfUfCAGfCfCfUfUfCAfCfUGfCGfCGfCGfCfUfCGAAfUAfCGfUfCfUAfUG | RT30FF_11 |
| | GfCAfCfCAfCGGfUfCGGAfUfCfCAfC (SEQ ID NO: 217) | |
| RT-FF12 | GGAGfCfUfCAGfCfCfUfUfCAfCfUGfCfCfCfUGfCAfUGfUGfCAAfUfCAfCfUGAG | RT30FF_12 |
| | GfCAfCfCAfCGGfUfCGGAfUfCfCAfC (SEQ ID NO: 218) | |
| RT-FFcg1 | GGAGfCfUfCAGfCfCfUfUfCAfCfUGfCfUfCfCGGGfCfCGAfCAAfUGfCGAfCfCG | RT30FF_164 |
| | AGGfUAfUfCAfCGGfCAfCfCAfCGGfUfCGGAfUfCfCAfC (SEQ ID NO: 219) | |
| RT-FFcg2 | GGAGfCfUfCAGfCfCfUfUfCAfCfUGfCGGfUfCGGGAfCfCfUGAfCfCAAfUfCfCG | RT30FF_231 |
| | GGAGfUfCAGfUGGfCAfCfCAfCGGfUfCGGAfUfCfCAfC (SEQ ID NO: 220) | |
| RT-FFcg3 | GGAGfCfUfCAGfCfCfUfUfCAfCfUGfCGfUAfCAfCAAfCGAAfCfUfUGfCfUGfUfU | RT30FF_433 |
| | fCfCGGGfUAGAGGfCAfCfCAfCGGfUfCGGAfUfCfCAfC (SEQ ID NO: 221) | |
| RT-FFcg4 | GGAGfCfUfCAGfCfCfUfUfCAfCfUGfCfUAAfCGGGfUfCfCGfUfUfUfCAAfUfCfCf | RT30FF_560 |
| | UfUAfCfCfUGfUfUGGGfCAfCfCAfCGGfUfCGGAfUfCfCAfC (SEQ ID NO: 222) | |
| RT-ubique | GGAGfCfUfCAGfCfCfUfUfCAfCfUGfCGGGAfUfUfCGfUGfUfUfUAfUfCfCfUfCAf | RT unfilter_all_2 |
| | CfUGAAfUAfCGfCfUGGGfCAfCfCAfCGGfUfCGGAfUfCfCAfC (SEQ ID NO: 223) | |
| 70N89 | GGGAAAAGCGAAUCAUACACAAGACCCAAGAGCUAACUUCCCGAAAGCAG | RT_nan |
| | AAAUAGCUGGGAGGCUUUUGGAGCGUCGUGGAUGCAUACCGUGCGGGCA | |
| | UAAGGUAUUUAAUUCCAUA (SEQ ID NO: 224) | |

1. A method for screening RNA aptamers, comprising following steps:
1) providing a library of RNA aptamers to be screened;
2) incubating the library of step 1) with a solid carrier fixed with a target, thereby inducing the RNA aptamer in the library to sufficiently bind to the target;
3) adopting a buffer gradient to elute the RNA aptamers bound to the target on the solid carrier in step 2), and collecting the eluate for each elution, respectively;
4) completely eluting the RNA aptamers still retained on the solid carrier after step 3), and collecting the eluate as the last group of eluate;
5) optionally concentrating and purifying the RNA aptamers in the eluates obtained in steps 3) and 4);
6) reverse-transcribing the RNA aptamers obtained in step 5) to obtain cDNAs;
7) amplifying and high-throughput sequencing the cDNAs obtained in step 6) to obtain sequencing data;
8) analysing the sequencing data obtained in step 7) and sorting RNA aptamer candidate sequences according to their binding potential from high to low, thereby obtaining high-affinity RNA aptamer sequences.

2. The method of claim 1, wherein the RNA aptamer library to be screened comprises preparing the RNA aptamer library in-house, purchasing the RNA aptamer library commercially, or obtaining the RNA aptamer library as a gift from another person.

3. The method of claim 1, wherein in step 2), after the RNA aptamer in the RNA aptamer library binds to the target, the solid carrier can be blocked to control and reduce non-specific background binding.

4. The method of claim 3, wherein the blocking refers to blocking the solid carrier with a non-target specific random RNA; or blocking the solid carrier with a target specific RNA.

5. The method of claim 1, wherein in step 2), the solid carrier includes, but is not limited to:
magnetic beads, matrix.

6. The method of claim 5, wherein the matrix includes, but is not limited to: agarose gel matrix, cephalosporin beads, nitrocellulose, polyvinylidene difluoride membranes, octyl alginate, and other carrier matrices.

7. The method of claim 1, wherein in step 2), the target is a small molecule, including but not limited to: steroids, dopamine, kanamycin, digoxin, antoxin, dinitroaniline, melamine, quinolone, aflatoxin; or a large molecule, including but not limited to: polypeptides, proteins (e.g., enzymes and antibodies, etc.) and complexes (proteins bound with RNA), macromolecules and compounds, and the like.

8. The method of claim 1, wherein in step 3), the gradient elution is an elution with a buffer of increased volume, or with a buffer of increased elution strength; preferably an elution with a buffer of increased volume.

9. The method of claim 8, wherein the buffer with increased elution strength is a buffer that prevents the RNA from folding to form a spatial structure by for example, increasing the concentration of salt ions or chelating agents.

10. The method of claim 8, wherein prior to the gradient elution, several background elutions are performed until the number of molecules of RNA aptamer contained in the eluate is not greater than 1% of the high throughput sequencing threshold.

11. The method of claim 10, wherein the volume of buffer for background elution should be not greater than the initial volume of buffer used for gradient elution.

12. The method of claim 1, wherein the elution may be a static elution (discontinuous elution, collecting the complete eluate at once) or a dynamic elution (continuous elution, continuously collecting a small amount of partial eluate), preferably a static elution.

13. The method of claim 1, wherein when a static elution is used, the last background elution is performed in a new vessel.

14. The method of claim 10, wherein when a static elution is used, the last background elution is performed in a new vessel.

15. The method of claim 10, wherein the buffer for background elution and the buffer for gradient elution may be the same or different; preferably the same.

16. The method of claim 1, wherein the buffer for the gradient elution comprises magnesium ions, preferably 5 mM magnesium ions, a pH below 8.5, preferably pH 7-8, and a concentration of NaCl or KCl between 75 mM-200 mM.

17. The method of claim 8, wherein after several gradient elutions such that the number of molecules of the RNA aptamer contained in the eluate is suitable for sequencing, and preferably the theoretical minimum of the number of molecules in the library is reduced to less than 10⁵, a complete elution is carried out in step 4), so that the RNA aptamers bound to the target on the solid carrier are completely eluted.

18. The method of claim 1, wherein the buffer for the complete elution contains reagents capable of releasing the RNA aptamer, including reagents capable of disrupting the binding of the target to the solid carrier, and/or reagents capable of disrupting the binding of the RNA aptamer to the target, and/or reagents directly disrupting the target.

19. The method of claim 1, wherein in step 7), a compensating sequence of 0-6 nt is randomly inserted between a sequencing linker and a cDNA constant region.

20. The method of claim 19, wherein in step 7), a custom-designed PhiX is introduced to further compensate the unbalanced base distribution in the constant region during the mixing of the multiple samples.

21. The method of claim 1, wherein in step 8), the binding potential means that the degree of enrichment increases fast in each eluate, rather than only considering the highest degree of enrichment.

22. The method of claim 21, wherein the binding potential is judged according to one or more of the following information about the RNA aptamer: the abundance of the RNA aptamer in each eluate, the number of times the RNA aptamer has been detected individually in each eluate, and the preference of the RNA aptamer to be present in subsequent eluates over the initial eluate.

23. The method of claim 22, wherein the above information is combined to fit a standard curve to judge the binding potential of the RNA aptamer according to the area under the curve (AUC).

24. The method of any one of claims 1-23, wherein the RNA aptamer comprises a chemically modified sequence; preferably, a fluorine-modified sequence.

25. An RNA aptamer, which is screened and obtained by using the method of any one of claims 1-24.

26. The RNA aptamer of claim 25, wherein the RNA aptamer comprises a RNA aptamer with known sequence and random modifications on different bases (e.g. A, U, G, C).

27. The RNA aptamer of claim 25, wherein the RNA aptamer does not comprise a conventional RNA aptamer with known sequence and no additional modifications.

28. The RNA aptamer of claim 25, wherein the RNA aptamer comprises a chemically modified sequence; preferably a fluorine modified sequence.

29. An apparatus for performing the method of any one of claims 1-24.

30. The apparatus of claim 29, wherein the apparatus comprises following modules:
1) a preparation module for preparing a library of RNA aptamers to be screened;
2) an incubating module for incudating the prepared library with a solid carrier (magnetic beads or matrix) fixed with a target;
3) an elution and collection module for performing a gradient elution to elute the RNA aptamers bound to the target on the solid carrier, and collecting the eluate for each elution, respectively;
4) an optional concentration and purification module for concentrating and purifying the RNA aptamer in the eluates;
5) a reverse-transcription module for reverse-transcribing the RNA aptamers to obtain CDNAs;
6) an amplification and high-throughput sequencing module for amplifying and high-throughput sequencing the cDNAs obtained above to obtain sequencing data; and
7) an analysis module for analysing said sequencing data and sorting the RNA aptamer candidate sequences according to their binding potential from high to low, thereby obtaining RNA aptamer sequences with high binding affinity.

31. A biochip comprising the RNA aptamers of any one of claims 25-28.

32. A method for preparing a biochip, comprising steps of:
1) screening and obtaining RNA aptamers using the method of any one of claims 1-24; and
2) preparing a biochip using the RNA aptamers screened and obtained in step 1).

33. A pharmaceutical composition comprising the RNA aptamers of any one of claims 25-28 and a pharmaceutically acceptable excipient or drug delivery carrier.

34. A drug delivery carrier, which is attached to the RNA aptamers of any one of claims 25-28.

35. The drug delivery carrier of claim 34, wherein the drug delivery carrier is a liposome.

36. A diagnostic reagent comprising the RNA aptamers of any one of claims 25-28 and other auxiliary reagents required for the diagnosis.

37. Use of the RNA aptamers screened and obtained by using the method of any one of claims 1-24 for preparing a biochip, a pharmaceutical composition or a diagnostic reagent.
